SEQUENCE LISTING (26 sequences)

SEQ ID NO: 1 - DNA, 24 bp, Artificial Sequence
  Exon 13
ctagaacggaagaaacccaagaca

SEQ ID NO: 2 - DNA, 24 bp, Artificial Sequence
  Exon 14 Fwd
ctgaccttgaccagcaacacacag

SEQ ID NO: 3 - DNA, 24 bp, Artificial Sequence
  Exon 14 Rev
agcaaatggagcgctacctgtcca

SEQ ID NO: 4 - DNA, 27 bp, Artificial Sequence
  Exon 15 Rev
ggattaccttatcaagcacatggtgac

SEQ ID NO: 5 - DNA, 19 bp, Artificial Sequence
  hIL10_-107 Fwd
ggaggagctctaagcagaa

SEQ ID NO: 6 - DNA, 19 bp, Artificial Sequence
  hIL10_-107 Rev
aagcccctgatgtgtagac

SEQ ID NO: 7 - DNA, 21 bp, Artificial Sequence
  hIL10_-336 Fwd
ctaggaacacgcgaatgagaa

SEQ ID NO: 8 - DNA, 21 bp, Artificial Sequence
  hIL10_-336 Rev
tttaggatgggctacctctct

SEQ ID NO: 9 - DNA, 22 bp, Artificial Sequence
  hIL10_-701 Fwd
gcagactactcttacccacttc

SEQ ID NO: 10 - DNA, 20 bp, Artificial Sequence
  hIL10_-701 Rev
tgtgttccaggctcctttac

SEQ ID NO: 11 - DNA, 22 bp, Artificial Sequence
  hIL10_-1032 Fwd
gaggaaagtaagggacctccta

SEQ ID NO: 12 - DNA, 22 bp, Artificial Sequence
  hIL10_-1032 Rev
cttctgtggctggagtctaaag

SEQ ID NO: 13 - DNA, 22 bp, Artificial Sequence
  hIL10_-1810 Fwd
agtttctagcaggctctttctc

SEQ ID NO: 14 - DNA, 21 bp, Artificial Sequence
  hIL10_-1810 Rev
ggtgtctcttcccaactcttc

SEQ ID NO: 15 - DNA, 24 bp, Artificial Sequence
  hIL10_-2920 Fwd
cttcagcaaatggcttgagataat

SEQ ID NO: 16 - DNA, 21 bp, Artificial Sequence
  hIL10_-2920 Rev
tgctgagattacaggcatgag

SEQ ID NO: 17 - DNA, 20 bp, Artificial Sequence
  hIL10_-4035  Fwd
gctctaatgggaggcagatt

SEQ ID NO: 18 - DNA, 21 bp, Artificial Sequence
  hIL10_-4035  Rev
gtgtgtgtgtgtgtgcttatg

SEQ ID NO: 19 - DNA, 21 bp, Artificial Sequence
  hIL10_-6004 Fwd
gtggagactggagcaaactaa

SEQ ID NO: 20 - DNA, 24 bp, Artificial Sequence
  hIL10_-6004 Rev
gtctgcatgactacctaggataac

SEQ ID NO: 21 - DNA, 19 bp, Artificial Sequence
  hIL10_-8050 Fwd
cgcctggaagagcactaat

SEQ ID NO: 22 - DNA, 22 bp, Artificial Sequence
  hIL10_-8050 Rev
tgaagaaaccactgtccttaca

SEQ ID NO: 23 - DNA, 20 bp, Artificial Sequence
  hIL10_-10218 Fwd
gacaggtcactggcaaatca

SEQ ID NO: 24 - DNA, 22 bp, Artificial Sequence
  hIL10_-10218 Rev
ccagggtggtctttcagtttag

SEQ ID NO: 25 - DNA, 22 bp, Artificial Sequence
  hIL10_-12113 Fwd
tcttctgatttccactgctctc

SEQ ID NO: 26 - DNA, 22 bp, Artificial Sequence
  hIL10_-12113 Rev
tgtatttccttctcgcaatcct

(19) United States
(12) Patent Application Publication
(10) Pub. No.: US 2024/0238340 A1
(43) Pub. Date: Jul. 18, 2024

(54) IMMUNOTHERAPY FOR INFLAMMATORY BOWEL DISEASE AND/OR CANCER

(71) Applicant: Rutgers, The State University of New Jersey, New Brunswick, NJ (US)

(72) Inventors: Derek B. Sant'Angelo, New Brunswick, NJ (US); Lisa K. Denzin, New Brunswick, NJ (US)

(73) Assignee: Rutgers, The State University of New Jersey, New Brunswick, NJ (US)

(21) Appl. No.: 18/558,644

(22) PCT Filed: Apr. 28, 2022
(86) PCT No.: PCT/US2022/026778

§ 371 Date: Nov. 2, 2023

Related U.S. Application Data
(60) Provisional application No. 63/183,357, filed on May 3, 2021.

(51) Int. Cl. A61K 35/17; A61K 39/00; A61P 35/00; A61P 37/06; C07K 14/47; C07K 14/54; C07K 16/28; C12N 15/85; G01N 33/68

(52) U.S. Cl. CPC A61K 35/17; A61K 39/4611; A61K 39/464452; A61P 35/00; A61P 37/06; C07K 14/4705; C07K 14/5428; C07K 16/2818; C12N 15/85; G01N 33/6893; A61K 2239/31; G01N 2333/4706; G01N 2800/065; G01N 2800/067; G01N 2800/50; G01N 2800/52

(57) ABSTRACT

The present disclosure includes compositions and methods for treating gastrointestinal inflammatory disease and/or cancers. In certain aspects, the disclosure includes an isolated cell comprising a nucleic acid vector comprising a gene encoding the transcription factor ZBTB20 which is operably linked to a promoter.

### CROSS-REFERENCE TO RELATED APPLICATION

The present application is entitled to priority under 35 U.S.C. § 119(e) to U.S. Provisional Patent Application No. 63/183,357 filed May 3, 2021, which is hereby incorporated by reference in its entirety herein.

### STATEMENT REGARDING FEDERALLY SPONSORED RESEARCH OR DEVELOPMENT

This invention was made with government support under R01AI122757 and R21AI120600 awarded by the National Institutes of Health. The government has certain rights in the invention.

### INCORPORATION-BY-REFERENCE OF MATERIAL SUBMITTED AS A TEXT FILE VIA THE OFFICE ELECTRONIC FILING SYSTEM

This invention contains one or more sequences in a computer readable format in an accompanying text file titled “370602-7051WO1 Sequence Listing_ST25,” created on Feb. 18, 2022, and which is 5.09 KB in size, the contents of which are incorporated herein by reference in their entirety.

### BACKGROUND

Regulatory T cells (Tregs) are a specialized population of T cells that function to suppress or regulate immune responses. As such, these cells play a vital role in maintaining immune homeostasis and self-tolerance. One key function of Tregs is the maintenance of tissue-specific tolerance, especially in the digestive tract. In that context, Tregs act to maintain the regulatory balance between recognition and elimination of pathogens and the tolerance of food substances and normal bacterial gut flora. Despite constant antigenic stimulation, controlled inflammatory responses and inflammation suppression predominate under normal conditions, wherein the gut immune system differentiates the antigenic signals from the high background noise of food and bacterial antigens.

Recent studies have identified distinct Treg subsets that coexist in the intestinal mucosa and mesenteric lymph nodes, and disturbances in Treg number and function are associated with immune-mediated disorders. Among the most common of these disorders are inflammatory bowel diseases (IBDs), of which there are several depending on location and severity. It is estimated that over 1.3% of adults in the U.S. are diagnosed with inflammatory bowel diseases each year, with the incidence increasing significantly over the past several decades. Also, adults suffering from IBD are more likely to have certain chronic health conditions including cardiovascular and respiratory diseases, cancer, arthritis, and kidney and liver diseases.

As such there is a need for therapies that can treat, ameliorate, and/or prevent diseases resulting from inappropriate inflammatory responses in gastrointestinal tissues by restoring normal immune regulation. Further, there is a need for therapies that can treat, ameliorate, and/or prevent certain cancers. The current invention addresses these needs.

### BRIEF SUMMARY OF THE DISCLOSURE

The present disclosure includes compositions and methods for treating gastrointestinal inflammatory disease based on subsets of CD4⁺ regulatory T cells (Tregs) which express high levels of the transcription factor ZBTB20.

Thus, in some aspects, the disclosure includes an isolated cell comprising a nucleic acid vector comprising a gene encoding the transcription factor ZBTB20 which is operably linked to a promoter.

In certain embodiments, the promoter is constitutive. In certain embodiments, the promoter is inducible.

In certain embodiments, the promoter drives the expression of ZBTB20 such that the function of the isolated cell is altered.

In certain embodiments, the expression of ZBTB20 results in enhanced IL-10 production by the isolated cell as compared to a cell not comprising the nucleic acid vector.

In certain embodiments, the cell is a T cell.

In certain embodiments, the T cell is a regulatory T cell.

In certain embodiments, the cell is derived from a mammal. In certain embodiments, the cell is derived from a mouse. In certain embodiments, the cell is derived from a human.

In another aspect, the disclosure includes a therapeutic composition comprising an effective amount of the isolated cell of any the above aspect or any other aspect or embodiments disclosed herein and a pharmaceutically acceptable carrier.

In another aspect, the disclosure includes a method for treating, ameliorating, and/or preventing an inflammatory disease in a subject in need thereof, the method comprising administering to the subject an effective amount of the isolated cell of any one of the above aspects or any aspect or embodiment disclosed herein, thereby treating, ameliorating, and/or preventing the inflammatory disease.

In certain embodiments, the inflammatory disease is a gastrointestinal inflammatory disease.

In certain embodiments, the gastrointestinal inflammatory disease is selected from the group consisting of inflammatory bowel disease (IBD), Crohn's disease, and ulcerative colitis.

In certain embodiments, the cells are administered via a route selected from the group consisting of intravenous, intraperitoneal, intramuscular, subcutaneous, and implantation.

In certain embodiments, the cells are autologous to the subject. In certain embodiments, the cells are heterologous to the subject.

In another aspect, the disclosure includes a method of treating, ameliorating, and/or preventing an inflammatory disease in a subject in need thereof, the method comprising:
a) isolating a cell from the subject,
b) contacting the cell with a nucleic acid vector encoding ZBTB20 such that expression of ZBTB20 protein is elevated in the cell as compared to uncontacted cells, thereby inducing an anti-inflammatory function in the cell, and
c) administering the contacted cell to the subject thereby treating, ameliorating, and/or preventing the inflammatory disease.

In certain embodiments, the inflammatory disease is a gastrointestinal inflammatory disease.

In certain embodiments, the gastrointestinal inflammatory disease is selected from the group consisting of inflammatory bowel disease (IBD), Crohn's disease, and ulcerative colitis.

In certain embodiments, the cell is a T cell.

In certain embodiments, the T cell is a regulatory T cell.

In certain embodiments, the anti-inflammatory function of the cell results from elevated expression of IL-10 by the cell.

In certain embodiments, the altered cells are administered via a route selected from the group consisting of intravenous, intraperitoneal, intramuscular, subcutaneous, and implantation.

In another aspect, the disclosure includes a method of determining the risk of developing an inflammatory disease in a subject, the method comprising:
a) obtaining a tissue sample from the subject,
b) assessing the level of ZBTB20 expression in a cell of the sample, and
c) comparing the level of ZBTB20 expression to a baseline expression level established from normal tissue which does not present the inflammatory disease;
   wherein ZBTB20 levels in the tissue sample that is lower than the baseline expression level represents an increased risk of the subject developing the inflammatory disease.

In certain embodiments, the inflammatory disease is a gastrointestinal inflammatory disease.

In certain embodiments, the inflammatory disease is selected from the group consisting of inflammatory bowel disease (IBD), Crohn's disease, and ulcerative colitis.

In another aspect, the disclosure provides a method of determining whether a cancer patient is a candidate for cancer treatment with anti-PD-1 therapy, the method comprising:
a. obtaining a tumor sample from the patient,
b. assessing the level of ZBTB20 expression in a cell of the tumor sample, and
c. comparing the level of ZBTB20 expression in the patient's tumor sample to a baseline expression level established from a tumor which was successfully treated with anti-PD-1 therapy;

In certain embodiments, if the patient's tumor sample has ZBTB20 levels that are lower than the baseline expression level, the patient is not a candidate for anti-PD-1 therapy.

In certain embodiments, the cancer is a solid tumor.

In certain embodiments, the cancer is selected from the group consisting of melanoma, head and neck cancer, non-small cell lung cancer, bladder cancer, and microsatellite unstable cancers.

In certain embodiments, the anti-PD-1 therapy is an antibody blockade therapy.

In certain embodiments, the antibody targets PD-1.

In certain embodiments, the antibody targets PD-L1.

In another aspect, the disclosure provides a method of immunotherapy for cancer for use in a patient in need thereof, the method comprising:
a. isolating an immune cell from the patient,
b. contacting the patient's immune cell with a nucleic acid vector encoding ZBTB20 such that expression of ZBTB20 protein is elevated in the patient's immune cell as compared to uncontacted immune cells, and
c. administering the contacted immune cell to the patient thereby treating or ameliorating the cancer.

In certain embodiments, the method further comprises administering to the subject anti-PD-1 therapy.

In certain embodiments, the patient has a better cancer treatment response to the anti-PD-1 therapy than in the absence of being administered the contacted immune cell.

In certain embodiments, the anti-PD-1 therapy is an antibody.

In certain embodiments, the anti-PD-1 therapy is an antibody specific for PD-1.

In certain embodiments, the anti-PD-1 therapy is an antibody specific for PD-L1.

In certain embodiments, the immune cell is a T cell.

In certain embodiments, the T cell is a CD4+ T cell.

In certain embodiments, the T cell is a CD8+ T cell.

In certain embodiments, the T cell is a mixture of CD4+ and CD8+ T cells.

In certain embodiments, the cancer is a solid cancer.

In certain embodiments, the cancer is selected from the group consisting of melanoma, head and neck cancer, non-small cell lung cancer, bladder cancer, and microsatellite unstable cancers.

### BRIEF DESCRIPTION OF THE DRAWINGS

The following detailed description of specific embodiments of the disclosure will be better understood when read in conjunction with the appended drawings. For the purpose of illustrating the disclosure, there are shown in the drawings exemplary embodiments. It should be understood, however, that the disclosure is not limited to the precise arrangements and instrumentalities of the embodiments shown in the drawings.

FIGS. 1A-1L illustrate that Zbtb20 is expressed in a subset T cells. FACS analysis of spleen cells from Zbtb20-eGFP (ZEG20) mice and non-GFP littermate. In ZEG20 mice, the endogenouse ZBTB20 promoter also drive expression of GFP, such that ZBTB20⁺ cells are also GFP⁺. (FIG. 1A) Expression of Zbtb20-GFP⁺ (ZEG20⁺) in CD3⁺ T cells. (FIG. 1B) Quantification of ZEG20⁺ T cells as a percent of all T cells and actual cell number. (FIG. 1C) Expression of CD4 and CD25 in ZEG20⁺ T cells. (FIG. 1D) Quantification of ZEG⁺ CD4⁺ T cells as a percent of GFP⁺ CD3⁺ T cells and actual cell number. (FIG. 1E) Quantification of ZEG20⁺ CD25⁺ CD4⁺ T cells as percent of GFP⁺ CD4⁺ T cells and actual cell number. (FIG. 1F) Expression of CD4, CD8, and CD25 in ZEG20⁻ T cells in ZEG20 mice. (FIG. 1G) Quantification of ZEG20⁻ CD4⁺ T cells as a percent of GFP⁻ CD3⁺ T cells and actual cell number. (FIG. 1H) Quantification of ZEG20⁻ CD25⁺ CD4⁺ T cells as a percent of GFP⁻ CD4⁺ T cells and actual cell number. (FIG. 1I) Expression of FoxP3 in ZEG20⁺ CD25⁺ CD4⁺ T cells and ZEG20⁺ CD25⁻ CD4⁺ T cells (left) and quantification (right). (FIG. 1J) Expression of FoxP3 in ZEG20⁻ CD25⁺ CD4⁺ T cells and ZEG20⁻ CD25⁻ CD4⁺ T cells (left) and quantification (right). (FIG. 1K) Comparison of FoxP3 expression in ZEG20⁺ and ZEG20-Tregs and (FIG. 1L) quantification (FoxP3-RFP MFI). Data representative of at least two independent experiments (n=3-7 mice/group). Significance represents p<0.05. Data analyzed using unpaired t-test and represented as mean±SEM.

FIGS. 2A-2H illustrate that Zbtb20 expressing T cells have a unique phenotype. FACS analysis of spleen cells from ZEG20 mice. (FIG. 2A) CD62L and Zbtb20 expression in Tregs collected from ZEG20;FoxP3-RFP mice. Representative FACS plot (left) and cell quantification (right). (FIG. 2B) Gene-expression profile of ZEG20⁺ Tregs and ZEG20⁻ CD62L^{lo} Tregs collected from the spleen of ZEG20;FoxP3-RFP mice. RNA-Seq data represented as fold-change difference in gene expression in ZEG20⁺ Tregs vs ZEG20⁻ CD62L^{lo} Tregs or ZEG20⁻ CD62L^{lo} Tregs vs ZEG20⁻ CD62L^{hi} Tregs (n=4 mice/group). (FIG. 2C-2H) Expression of CD62L, CD44, TIGIT, GITR, ICOS, and Neuropilin-1 in subsets of Tregs collected from ZEG20 mice. Data representative of at least two independent experiments (n=4-5 mice/group). Significance represents p<0.05. Data analyzed using unpaired One-way ANOVA and represented as mean±SEM.

FIGS. 3A-3J illustrate that Zbtb20 expressing T cells constitutively transcribe Il10. (FIG. 3A) CD62L expression in ZEG20⁺ and ZEG20⁻ CD3⁺ thymocytes assessed by FACS. Representative FACS plot (left) and quantification (right). (FIG. 3B) Il10 gene expression in subsets of ZEG20⁺, ZEG20⁻ CD62L^{lo}, and ZEG20⁻ CD62L^{hi} (CD25⁺) Tregs and (FIG. 3C) in ZEG20⁺, ZEG20⁻ CD62L^{lo}, and ZEG20⁻ CD62L′ (CD25⁻) CD4⁺ T cells sorted from the spleen of ZEG20 mice assessed by qPCR. (FIG. 3D) Il10 gene expression in ZEG20⁺ T cells and ZEG20⁻ T cells sorted from the thymuses of ZEG20 mice assessed by qPCR. (FIG. 3E) IL-10 cytokine concentration in medium collected after 24 h PMA and ionomycin activation of different subsets of Tregs and CD4⁺ T cells sorted from the spleens of ZEG20 mice. (FIG. 3F) Rapid secretion of IL-10 by different subsets of Tregs and CD4⁺ T cells after 3 h activation with PMA and ionomycin (the IL-10 secreting cells are stained with IL-10 catch antibodies and APC detection reagent). (FIG. 3G) The Zbtb20 staining of Tregs' (left) and CD4⁺ T cells' subsets (right) sorted from spleens of IL-10^{ires-GFP} mice. The cells were stained with an anti-Zbtb20-PE antibody and assessed by FACS. (FIG. 3H) Surface expression of CD44, (FIG. 3I) GITR, (FIG. 3J) TIGIT in Tregs sorted from ZEG20 mice (left), and IL-10^{ires-GFP} mice (right). Data representative of at least three independent experiments (n=3-5 mice/group). Significance represents p<0.05. Data analyzed using unpaired t-test or One-way ANOVA and represented as mean±SEM.

FIGS. 4A-4I illustrate that Zbtb20⁺ Tregs are enriched in the gastrointestinal tract. FACS analysis of single cell suspensions of spleens, PPs, epithelium, and LP from ZEG20;FoxP3-RFP mice. (FIG. 4A) Zbtb20-GFP and CD62L expression in Tregs in spleen and PPs. (FIG. 4B) Zbtb20-GFP and CD62L expression in IEL (sIEL) and LPL (sLPL) Tregs in small intestine. (FIG. 4C) Zbtb20-GFP and CD62L expression in IEL (cIEL) and LPL (cLPL) Tregs in the colon. (FIG. 4D) The percentage of ZEG20*Tregs in the spleen, PPs, epithelium, and LP of the small intestine and the colon. (FIG. 4E) The body weight change in ZEG20 mice that received 3% DSS or water. (FIG. 4F) Representative FACS plots of ZEG20⁺ CD3⁺ T cells collected from the epithelium of ZEG20 mice that received water (left) or 3% DSS (right). (FIG. 4G) The percentage (left) and cell number (right) of ZEG20⁺ CD3⁺ T cells in the epithelium of ZEG20 mice that received water or 3% DSS. (FIG. 4H) The representative FACS plots of ZEG20⁺ CD3⁺ T cells collected from LP of ZEG20 mice that received water or 3% DSS. (FIG. 4I) The percentage (left) and cell number (right) of ZEG20⁺ CD3⁺ T cells in LP of ZEG20 mice that received water or 3% DSS. Data representative of at least three independent experiments (n=5-7 mice/group). Significance represents p<0.05. Data analyzed using unpaired t-test or One-way ANOVA and represented as mean±SEM.

FIGS. 5A-5I illustrate that deletion of zbtb20 in T cells impacts intestinal homeostasis. (FIG. 5A) Expression of Zbtb20 in ZEG20⁺ Tregs and ZEG20⁻ CD62L^{lo} Tregs sorted from zbtb20^{fl/fl};ZEG20 (WT;GFP) mice and Zbtb20^{wannabe} Tregs sorted from zbtb20-cKO;ZEG20 mice. The cells were made permeable, stained with an anti-Zbtb20-PE antibody, and assessed by FACS. (FIG. 5B) Expression of Zbtb20 in ZEG20⁺ CD25⁻ CD4⁺ T cells and ZEG20⁻ CD62L^{lo} CD25⁻ CD4⁺ T cells sorted from zbtb20^{fl/fl};ZEG20 (Wt;GFP) mice and Zbtb20^{wannabe} CD25⁻ CD4⁺ T cells sorted from zbtb20-cKO;ZEG20 mice. The cells were stained with an anti-Zbtb20-PE antibody and assessed by FACS. (FIG. 5C) The percent and absolute number of ZEG20⁺ and Zbtb20^{wannabe} T cells in the epithelium and (FIG. 5D) LP of zbtb20^{fl/fl};ZEG20 and zbtb20-cKO;ZEG20 mice (respectively). (FIG. 5E) A total number of leukocytes in PPs, (FIG. 5F) colonic epithelium and (FIG. 5G) colonic LP of zbtb20^{fl/fl};ZEG20 and zbtb20-cKO ZEG20 mice. (FIG. 5H) A number of Peyer's patches in the small intestine of zbtb20^{fl/fl};GFP and zbtb20-cKO;GFP mice. (FIG. 5I) The percent and absolute number of ZEG20⁺ and Zbtb20^{wannabe} T cells in PPs of zbtb20^{fl/fl};ZEG20 and zbtb20-cKO;ZEG20 mice (respectively). Data representative of at least two independent experiments (n=5-6 mice/group). Significance represents p<0.05. Data analyzed using unpaired t-test and represented as mean±SEM.

FIGS. 6A-6J illustrate that the conditional deletion of zbtb20 in T cells changes the phenotype of Zbtb20^{wannabe} T cells. (FIG. 6A) Expression of CD62L and CD44 in ZEG20⁺ and Zbtb20^{wannabe} Tregs; representative FACS plots. (FIG. 6B) The percent of CD62L^{lo} CD44^{hi} ZEG20⁺ and CD62L^{lo} CD44^{hi} Zbtb20^{wannabe} Tregs in the spleen of zbtb20^{fl/fl};ZEG20 and zbtb20-cKO;ZEG20 mice (left). The percent of CD62L^{hi} CD44^{hi} ZEG20⁺ and CD62L^{hi} CD44^{hi} Zbtb20^{wannabe} Tregs in spleen of zbtb20^{fl/fl};ZEG20 and zbtb20-cKO;ZEG20 mice (right). (FIG. 6C) Expression of CD62L and (FIG. 6D) CD44 in ZEG20⁺ and Zbtb20^{wannabe} Tregs. (FIG. 6E) IL-10 cytokine concentration in medium collected after 24 h PMA and ionomycin activation of ZEG20⁺ and Zbtb20^{wannabe} T cells; cells were collected from zbtb20^{fl/fl};ZEG20 and zbtb20-cKO;ZEG20 mice (respectively). (FIG. 6F) Rapid secretion of IL-10 by Tregs collected from 8-week old zbtb20^{fl/fl} (Wt) and zbtb20-cKO mice. Cells were activated for 3 h with PMA and ionomycin and the IL-10 secreting cells were stained with IL-10 capture antibodies and APC detection reagent. (FIG. 6G) Representative image of a colon collected from zbtb20^{fl/fl} (WT) and zbtb20-cKO mice (left) and their normalized length (right). (FIG. 6H) Representative images of H&E staining of colons collected from naïve 8-week old Zbtb20^{fl/fl} (WT; left) and Zbtb20 cKO mice (right); Images at 10× (scale bar=200 mm) and 40× (insert) magnification. (FIG. 6I) The histological and inflammation score assessed in colons collected from naïve 8-week old Zbtb20^{fl/fl} (Wt) and Zbtb20 cKO mice. (FIG. 6J) The concentration of creatinine, FITC-dextran (FD4), and Rhodamine B-dextran (RD70) in the serum of naïve 14-week old Zbtb20^{fl/fl} (Wt) and Zbtb20 cKO mice. Serum collected 5 h after mice were gavaged with all three probes. Data represent at least two independent experiments (n=4-5 mice/group). Significance represents p<0.05. Data analyzed using unpaired t-test and represented as mean±SEM.

FIGS. 7A-7K illustrate that mice with the targeted deletion of zbtb20 develop more severe symptoms of DSS-induced colitis. (FIG. 7A) Body weight change of zbtb20^{fl/fl} (WT) and zbtb20-cKO mice that received 3% DSS for 5 days to induce colitis; the red cross indicates the presence of occult blood in the stool, the double red crosses indicate the presence of visible blood in the stool. (FIG. 7B) Survival of zbtb20^{fl/fl} (WT) and zbtb20-cKO mice with DSS-induced colitis. (FIG. 7C) The length of colons collected from zbtb20^{fl/fl} (WT) and zbtb20-cKO mice with DSS-induced colitis; A representative image of the colons (left) and their normalized length (right). (FIG. 7D) Representative images of H&E staining of colons collected from zbtb20^{fl/fl} (WT; left) and zbtb20-cKO (right) mice with DSS-induced colitis; images at 10× magnification (scale bar=200 mm). (FIG. 7E) Histological and inflammation score of colons of zbtb20^{fl/fl} (WT) and zbtb20-cKO collected at day 9 post-induction of colitis with 3% DSS. (FIG. 7F) The percent and the absolute number of MHC II⁺ cells in the epithelium and (FIG. 7G) LP of colons collected from zbtb20^{fl/fl};ZEG20 and zbtb20-cKO;ZEG20 mice (respectively) that received regular drinking water or 3% DSS to induce colitis. (FIG. 7H) Body weight change due to DSS-induced colitis; the zbtb20 cKO mice received i.p. injection of 500 k of total Tregs collected from the spleen of healthy WT mouse a day before induction of the colitis; the control zbtb20^{fl/fl} (WT) and zbtb20-cKO mice received the vehicle (* indicates significance between “Wt+PBS vs cKO+PBS”; ⁺ indicates significance between “cKO+ Tregs Wt vs cKO+PBS”). (FIG. 7I) Survival of zbtb20^{fl/fl} (WT) and zbtb20-cKO mice with DSS-induced colitis after receiving i.p. injection with 500 k of healthy Tregs or the vehicle. (FIG. 7J) Body weight change due to DSS-induced colitis; the zbtb20 cKO mice received i.p. injection of 100 k of sorted ZEG20⁺ Tregs or ZEG20⁻ CD62L^{lo} Tregs collected from the spleen of healthy ZEG20;Foxp3-RFP mouse a day before induction of the colitis; the control zbtb20^{fl/fl} (WT) and zbtb20-cKO mice received the vehicle (^{#} indicates significance between “cKO+GFP⁺ and cKO+GFP⁻”; * indicates significance between “cKO+GFP⁺ vs cKO+PBS”; ⁺ indicates significance between “WT+PBS vs cKO+PBS”). (FIG. 7K) Survival of zbtb20^{fl/fl} (WT) and zbtb20-cKO mice with DSS-induced colitis after receiving i.p. injection with 100 k of sorted ZEG20⁺ Tregs or ZEG20⁻ CD62L^{lo} Tregs or the vehicle. Data represent at least two independent experiments (n=5-10 mice/group). Significance represents p<0.05. Data analyzed using unpaired t-test or One-way ANOVA and represented as mean±SEM.

FIGS. 8A-8F illustrate that Zbtb20⁺ T cells suppress the myeloid cells during DSS-induced-colitis. (FIG. 8A) Body weight change due to DSS-induced colitis; the zbtb20-cKO mice received i.p. injection of 500 k of total Tregs collected from the spleen of zbtb20^{fl/fl};ZEG20 (WT) or zbtb20-cKO;ZEG20 (cKO) mice a day before induction of the colitis; the control zbtb20^{fl/fl} and zbtb20-cKO mice received the vehicle (^{#} indicates significance between “cKO+ Tregs WT and cKO+PBS”; ⁺ indicates significance between “cKO+ Tregs KO vs cKO+PBS; * indicates significance between “WT+PBS vs cKO+PBS). (FIG. 8B) Survival of zbtb20^{fl/fl} (WT) and zbtb20-cKO mice with DSS-induced colitis after receiving i.p. injection with 500 k of total Tregs collected from the spleen of zbtb20^{fl/fl};ZEG20, zbtb20-cKO;ZEG20 mice, or the vehicle. (FIG. 8C) The concentration of IL-6 and (FIG. 8D) IL-1α in the serum of zbtb20-cKO mice that received i.p. injection of 500 k of total Tregs collected from the spleen of zbtb20^{fl/fl};ZEG20 or zbtb20-cKO;ZEG20 mice a day before induction of the colitis, and zbtb20^{fl/fl} (WT) and zbtb20-cKO mice received the vehicle. (FIG. 8E) The concentration of IL-6, IL-9, and IL-17 in the serum of zbtb20^{fl/fl} (WT) and zbtb20-cKO mice collected 21 h after i.p injected with 100 g LPS. (FIG. 8F) Body weight change due to the colitis induced with anti-CD40 agonist antibody. The zbtb20^{fl/fl} (WT), zbtb20-cKO, and RAG1 KO mice were i.p. injected with 100 μg of an anti-CD40 agonist antibody or isotype control (* indicates significance between “Wt+aCD40 vs zbtb20-cKO+aCD40”). Data represent at least two independent experiments (n=3-6 mice/group). Significance represents p<0.05. Data analyzed using unpaired t-test or One-way ANOVA and represented as mean±SEM.

FIGS. 9A-9D illustrate specific expression of GFP in cells known to express Zbtb20. (FIG. 9A) zbtb20 expression in sorted GFP⁺ and GFP⁻ T cells collected from the spleen of ZEG20 mice was assessed by rtPCR; the PCR products were run on 1% agarose gel and (3-actin was used as a housekeeping gene. (FIG. 9B) Western Blots for Zbtb20 expression in transfected HELA cells, T cells ectopically expressing zbtb20, and sorted ZEG20⁺ and ZEG20⁻ T cells were blotted. Two independent experiments are shown. (FIG. 9C) Zbtb20 expression in CD19⁺ B cells from the peritoneal cavity of a ZEG20 mouse. B1 cells were identified as CD5⁺, CD43⁺, IgM^{hi}. (FIG. 9D) Fas expression in GFP⁺ and GFP⁻ CD19⁺ B cells. GFP⁺ cells expressed FAS indicating they are germinal center B cells, while GFP⁻ cells did not express FAS. Data represent at least two independent experiments (n=3-6 mice/group).

FIGS. 10A-10G illustrate the phenotype of GFP⁺ T cells in spleen and thymus of ZEG20 and IL-10-GFP mice. (FIG. 10A) Representative image of FACS plots. Zbtb20 expression in T cells of non-GFP (left) and ZEG20 (right) littermates. (FIG. 10B) Expression of CD4 and CD25 in ZEG20⁺ T cells. (FIG. 10C) Expression of FoxP3 in ZEG20⁺ CD25⁺ CD4⁺ T cells and ZEG20⁺ CD25⁻ CD4⁺ T cells; Cells were permeabilized and stained with anti-FoxP3-APC antibody. (FIG. 10D) Zbtb20 expression in CD3⁺ thymocytes of ZEG20;FoxP3-RFP mice. Representative FACS plot (left) and quantification (right). (FIG. 10E) Expression of CD4 and CD25 in ZEG20⁺ CD3⁺ thymocytes. (FIG. 10F) Expression of FoxP3 in ZEG20⁺ CD25⁺ CD4⁺ T cells and ZEG20⁺ CD25⁻ CD4⁺ T cells. (FIG. 10G) Representative FACS plot showing IL10-GFP and CD3 expression in splenocytes collected from IL-10-GFP mouse. Data represent at least two independent experiments (n=3-6 mice/group).

FIGS. 11A-11H illustrate the phenotype of ZEG20⁺ T cells in spleen and thymus of ZEG20 mice. (FIG. 11A) CD62L and Zbtb20 expression FoxP3⁻ CD4⁺ T cells collected from ZEG20;FoxP3-RFP mice. Representative FACS plot and cell quantification. (FIG. 11B) Gene-expression profile of ZEG20⁺ Tregs and ZEG20⁻ CD62L^{lo} Tregs collected from the spleen of ZEG20;FoxP3-RFP mice. RNA-Seq data represented as log 2 fold-change difference in gene expression in ZEG20⁺ Tregs vs ZEG20⁻ CD62L^{hi} Tregs or ZEG20-CD62L^{lo} Tregs vs ZEG20⁻ CD62L^{hi} Tregs (n=4 mice/group). (FIG. 11C) Expression of CD62L, (FIG. 11D) CD44, (FIG. 11E) TIGIT, (FIG. 11F) GITR, (FIG. 11G) ICOS, and (FIG. 11H) Neuropilin-1 in subsets of CD4⁺ T cells collected from ZEG20 mice. Data represent at least two independent experiments (n=4-5 mice/group). Significance represents p<0.05. Data analyzed using unpaired one-way ANOVA and represented as mean±SEM.

FIGS. 12A-12B illustrate in vitro and in vivo induction of zbtb20. (FIG. 12A) In vitro induction of zbtb20 in GFP⁻ CD4⁺ T cells collected from ZEG20 mice. The cells were cultured in the presence of TGFβ, IL-6 or both for 72 h and analyzed by FACS. (FIG. 12B) In vivo induction of zbtb20 in GFP⁻ CD4⁺ T cells collected from ZEG20 mice. Naïve GFP⁻, CD62L^{hi} CD4⁺ CD45.2⁺ T cells were i.p. injected into the SJL (CD45.1+) mice. The expression of Zbtb20-GFP in Payer's patches was assessed after 2 weeks by FACS. Data represent at least two independent experiments (n=3-6 mice/group).

FIGS. 13A-13H illustrate an analysis of zbtb20-cKO;GFP mice. (FIG. 13A) Deletion of a “floxed” exon in ZEG20;CD4-Cre;zbtb20^{fl/fl} (zbtb20-cKO;ZEG20) mice. The mRNA was isolated from sored Zbtb20^{wannabe} CD4⁺ T cells collected from the spleen of zbtb20-cKO;ZEG20 mice and ZEG20⁺ CD4⁺ T cells collected from the spleen of zbtb20^{fl/f};ZEG20 mice. The expression of zbtb20 was assessed by PCR using 3 sets of primes; the PCR products ran on 1% agarose gels. (FIG. 13B) A total number of leukocytes in the thymus and spleen of zbtb20^{fl/f}/GFP and zbtb20-cKO/GFP mice. (FIG. 13C) Percent and an absolute number of ZEG20⁺ and Zbtb20^{wannabe} T cells in the thymus of zbtb20^{fl/f}; ZEG20 and zbtb20-cKO;ZEG20 mice (respectively). (FIG. 13D) A total number of leukocytes in the spleen of zbtb20^{fl/f};ZEG20 and zbtb20-cKO;ZEG20 mice. (FIG. 13E) Expression of GITR, (FIG. 13F) TIGIT, and (FIG. 13G) ICOS in ZEG20⁺ and Zbtb20^{wannabe} Tregs and quantitation of expression. (FIG. 13H) The ratio of Creatinine to Rhodamine B-dextran (Creatinine/RD70) (left) and the ratio of FITC-dextran to Rhodamine B-dextran (FD4/RD70) (right); concentrations of Creatinine, FD4, and RD70 were assessed in the serum of naïve 14-week old zbtb20^{fl/fl} and zbtb20-cKO mice. Serum collected 5 h after mice were gavaged with the probes. Data represent at least two independent experiments (n=3-6 mice/group). Significance represents p<0.05. Data analyzed using unpaired t-test and represented as mean SEM.

FIGS. 14A-14E illustrate DSS-induced colitis in zbtb20-cKO and ZEG20 mice. DSS-induced colitis in zbtb20-cKO and ZEG20 mice (FIG. 14A) Representative image of occult blood in the stool collected from zbtb20-cKO and ZEG20 mice and detected with the Hemoccult II test. (FIG. 14B) Percent and an absolute number of ZEG20⁺ and Zbtb20^{wannabe} T cells in the epithelium of zbtb20^{fl/fl}/GFP and zbtb20-cKO/GFP mice (respectively) that received regular drinking water or 3% DSS to induce colitis. (FIG. 14C) Percent and an absolute number of ZEG20⁺ and Zbtb20^{wannabe} T cells in LP of zbtb20^{fl/fl}/GFP and zbtb20-cKO;ZEG20 mice (respectively) that received regular drinking water or 3% DSS to induce colitis. (FIG. 14D) Percent and an absolute number of CD3⁺ T cells in the epithelium and (FIG. 14E) LP of colons collected of zbtb20^{fl/fl};ZEG20 and zbtb20-cKO;ZEG20 mice (respectively) that received regular drinking water or 3% DSS to induce colitis. Data represent four independent experiments (n=5 mice/group). Significance represents p<0.05. Data analyzed using unpaired One-way ANOVA and represented as mean±SEM.

FIG. 15 illustrates the transduction of peripheral mouse CD4⁺ T cells to express ZBTB20. Dot plots indicate that the vector expresses GFP and can be used to monitor the efficiency of transduction. T cells were transduced with “empty” vector (GFP only) or Zbtb20 expressing vector. Final amount of virus used was 25 ul and 40 ul.

FIGS. 16A-16F illustrate phenotypic changes in peripheral mouse CD4⁺ T cells transduced to express ZBTB20. (FIGS. 16A-16B) The level of expression of TIGIT (FIG. 16A) or IL-10 (FIG. 16B) (light blue) for T cells transduced with Zbtb20 as compared to cells transduced with empty vector (red). (FIGS. 16C-16D) The level of expression of ICOS (FIG. 16C) or Nrp1 (FIG. 16D) (light blue) for T cells transduced with Zbtb20 as compared to cells transduced with empty vector (red). (FIG. 16E-16F) The level of expression of GITR (FIG. 16E) or CD25 (FIG. 16F) (light blue) for T cells transduced with Zbtb20 as compared to cells transduced with empty vector (red). Graphs below each histogram represent percentage of GFP positive cells in ZBTB20 or empty vector control cells at each concentration of virus.

FIG. 17 illustrates quantification of CD62L and Zbtb20 expression in Tregs from the spleen of ZEG20;FoxP3-RFP mice. Percent of the ZEG20⁺ and ZEG20⁻ populations among CD62L^{lo} Tregs is shown in the graph.

FIGS. 18A-18E illustrate that Zbtb20-expressing T cells constitutively express Il10. FIG. 18A IL-10 expression by CD4SP Thymocytes detected by cell surface capture reagent after 3 h activation with PMA/ion. FIG. 18B Expression of IL-10 and Helios in ZEG20⁺ and ZEG20⁻ thymocytes after 3 h activation with PMA/ion. FIG. 18C Zbtb20 protein expression in IL-10-GFP⁺ and IL-10 GFP-Tregs and CD4⁺ T cells sorted from spleens of IL-10^{ires-GFP} mice. FIG. 18D Expression of Zbtb20 in MT-2 cells assessed by Western blot. FIG. 18E ChIP of Zbtb20 bound to the IL10 promoter in MT-2 cells. The X-axis indicates 11 regions identified in the ENCODE database as accessible to transcription factor binding (the indicated base pair number is the position of the 5′ primer used for PCR, see Table 1), and the Y-axis shows the fold enrichment at each site as compared to an IP with an irrelevant antibody (the dotted line). The shaded bars indicate regions that had significantly enriched binding of Zbtb20, based on three independent ChIP experiments. For FACS, qPCR and cytokine release at least three independent experiments were performed. FIG. 18A, 18C total n=3 mice/group; FIG. 18B total n=4/group. Significance represents p<0.05. Data were analyzed using an unpaired t-test (FIG. 18B) or One-way ANOVA with Tukey correction (FIG. 18E) and represented as mean SEM.

FIGS. 19A-19C illustrate that Zbtb20-expressing Tregs are enriched in the gastrointestinal tract. FIG. 19A. IL-10 expression by the indicated subsets detected by cell surface capture after 3 h activation with PMA/ion. FIG. 19B Representative FACS plots and quantification of the increase in ZEG20⁺ CD4⁺ T cells among the cIEL and FIG. 19C the cLPL from ZEG20;FoxP3-RFP mice following DSS treatment. Three independent experiments were performed. A,B,C,D total n=5-7 mice/group; E total n=4 mice/group; F,G total n=6 mice/group. Significance represents p<0.05. Data were analyzed using an unpaired t-test and represented as mean±SEM.

FIG. 20 illustrates that the percent and the absolute number of total CD11b⁺, F4/80⁺ macrophages and proinflammatory CD11b⁺, F4/80⁺, CD80⁺ macrophages in the LP of colons collected from Zbtb20^{fl/fl} WT and Zbtb20-cKO mice with DSS-induced colitis.

FIG. 21 illustrates that loss of body weight and survival of WT and cKO mice that received an i.p. injection of PBS or 100 k of sorted ZEG20⁺ IL-10^{+/+} Tregs or ZEG20⁺ IL-10^{-/-} Tregs collected from the spleen of healthy ZEG20 mice or IL10 deficient ZEG20 mice one day before induction of the colitis (^{a} indicates significance between cKO+ZEG20⁺ Tregs and cKO+PBS; ⁺ indicates significance between cKO+ZEG20⁺ Tregs and cKO+ZEG20 IL-10^{-/-} Tregs; ^{#} indicates significance between WT+PBS and cKO+ZEG20 IL-10^{-/-} Tregs; * indicates significance between WT+PBS and cKO+PBS). At least two independent experiments were performed. Total n=6-9 mice/group. Significance represents p<0.05. Data were analyzed using two-way ANOVA with Tukey correction and represented as mean SEM.

FIGS. 22A-22E illustrate Zbtb20 expression during thymic development. Expression of Nrp-1 in the indicated subsets of (FIG. 22A) T cells from the spleen of ZEG20;FoxP3-RFP mice. FIG. 22B. Expression of Zbtb20 in CD3^{hi} CD4SP and CD8SP and CD3^{lo} DP thymocytes from ZEG20 mice. FIG. 22C. Expression of CD73 on ZEG20⁺ CD4SP thymocytes. FIG. 22D. Expression of CD25 and FoxP3 in ZEG20⁺ CD4SP thymocytes. FIG. 22E. Expression of CD62L on ZEG20⁺ CD4SP thymocytes. At least two independent experiments were performed. FIGS. 22A, 22C, 22D total n=5 mice/group. FIGS. 22B, 22E total n=3 mice/group. Significance represents p<0.05. Data were analyzed using One-way ANOVA and represented as mean±SEM

FIGS. 23A-23B illustrate validation of Zbtb20-eGFP reporter. FIG. 23A. Zbtb20 expression in sorted GFP⁺ and GFP⁻ T cells collected from the spleen of 3 ZEG20 mice assessed by qPCR. 500×10³ cells were used for mRNA isolation and RT reaction FIG. 23B. Zbtb20 expression in sorted GFP⁺ and GFP⁻ T cells collected from the spleen of 4 ZEG20;FoxP3-RFP mice was assessed by RNA-Seq and presented as reads per kilobase per million mapped reads (RPKM).

FIG. 24 illustrates FACS analysis of ZEG20⁺ and ZEG20⁻ Tregs with antibodies against TCR variable regions (Vα2, Vα3.2, Vα8, Vβ6, Vβ7, Vβ8). At least two independent experiments were performed with a total n=3 mice/group. Significance represents p<0.05. Data were analyzed using an unpaired t-test and represented as mean±SEM.

FIGS. 25A-25E illustrate IL-10 and Zbtb20 expression in different subsets of cells. FIG. 25A. Percentage of Helios expressing in the indicated spleen T cell populations. FIG. 25B. IL-10 and Helios in ZEG20⁺, FoxP3⁻ ZEG20⁻ CD62L^{lo}, and FoxP3⁻ ZEG20⁻ CD62L^{hi} CD4⁺ spleen T cells after 3 h activation with PMA/ion. FIG. 25C Representative FACS plot showing IL10-GFP in CD3+ spleen T cells from IL-10^{ires-GFP} mouse. FIG. 25D. Expression of CD44, GITR, and TIGIT on GFP⁺ and GFP⁻ Tregs from ZEG20 mice and IL-10^{ires-GFP} mice. FIG. 25E. Zbtb20 expression in MT-2 cells assessed by FACS. At least three independent experiments were performed. FIGS. 25A, 25B total n=4 mice/group; FIGS. 25C,25D total n=3 mice/group. Significance represents p<0.05. Data were analyzed using a one-way ANOVA with Tukey correction and represented as mean±SEM.

FIG. 26A-26E illustrate analysis of Zbtb20-cKO/GFP mice. FIG. 26A. Expression of Zbtb20 in ZEG20⁺ Tregs and ZEG20⁻ CD62L^{lo} Tregs sorted from Zbtb20^{fl/fl}/ZEG20 (WT/GFP) mice and Zbtb20^{wannabe} Tregs sorted from Zbtb20-cKO/ZEG20 mice (cKO/GFP). The cells were made permeable, stained with an anti-Zbtb20-PE antibody, and assessed by FACS. FIG. 26B. Expression of TIGIT in ZEG20⁺ and Zbtb20^{wannabe} Tregs collected from spleens. FIG. 26C. Expression (MFI) of CD62L and CD44 in ZEG20⁺ and Zbtb20^{wannabe} Tregs in spleens. FIG. 25D. The percent and absolute number of ZEG20⁺ and Zbtb20^{wannabe} T cells in the colon epithelium and LP. FIG. 26E. Representative image of a colon collected from Zbtb20^{fl/f}/GFP (WT) and Zbtb20-cKO (cKO) mice (left) and their normalized length (right). At least two independent experiments. FIG. 26A. total n=4 mice/group; FIGS. 26B, 26C, 26D total n=5 mice/group; FIG. 26E total n=4 mice/group. Significance represents p<0.05. Data were analyzed using an unpaired t-test and represented as mean±SEM.

FIGS. 27A-27E illustrate DSS-induced colitis in Zbtb20-cKO (CD4-Cre;cKO), FoxP3-Cre/Zbtb20^{fl/fl} (FoxP3-Cre; cKO), and ZEG20 mice. FIG. 27A. The absolute numbers of ZEG20⁺ and Zbtb20^{wannabe} T cells in the colon epithelium and LP of WT and cKO mice that received regular drinking water or 3% DSS to induce colitis. FIG. 27B. The absolute numbers of non-Zbtb20 CD3⁺ T cells in the colon epithelium and LP of WT and cKO mice that received regular drinking water or 3% DSS to induce colitis. FIG. 27C. The concentration of creatinine, FITC-dextran (FD4), and Rhodamine B-dextran (RD70) in the serum of naïve 14-week old Zbtb20^{fl/fl} (WT), Zbtb20-cKO (CD4-Cre;cKO), and FoxP3-Cre/Zbtb20^{fl/fl} (FoxP3-Cre;cKO) mice and the ratios of Creatinine to Rhodamine B-dextran (Creatinine/RD70) and FITC-dextran to Rhodamine B-dextran (FD4/RD70). FIG. 27D. Loss of body weight and survival of WT, CD4-Cre;cKO, and FoxP3-Cre;cKO mice following induction of colitis with DSS. (* indicates significance between CD4-Cre;cKO and WT; ⁺ indicates significance between CD4-Cre;cKO and FoxP3-Cre;cKO; ^{#} indicates significance between FoxP3-Cre;cKO and WT). FIG. 27E. The length of the colons collected from WT, CD4-Cre;cKO, and FoxP3-Cre;cKO mice with DSS-induced colitis. At least two independent experiments were performed. FIGS. 27A, 27B total n=5 mice/group; FIG. 27C total n=4 mice/group; FIG. 27D total n=9 mice/group; FIG. 27E total n=4-9 mice/group. Significance represents p<0.05. Data were analyzed using one-way ANOVA with Tukey correction and represented as mean±SEM.

FIG. 28 illustrates Adoptive transfer of ZEG20 and non-ZEG20 Tregs. Loss of body weight and survival of WT and cKO mice that received an i.p. injection of PBS or 500 k of ZEG20⁺ or ZEG20⁻ Tregs collected from the spleen of healthy ZEG20 mice one day before induction of the colitis. Data show one experiment with an n=3.

FIGS. 29A-29B illustrate Zbtb20 expression during thymic development. FIG. 29A. Nearly half of the GFP expressing CD3^{lo} DP thymocytes are CD24^{hi}. FIG. 29B. Expression of Zbtb20-GFP in CD4 and CD8 T cells collected from fetal thymic organ cultures (FTOC) was done with E16 organs taken from ZEG20 or control embryos and cultured for 15 days. Three independent experiments were performed. A total n=5 mice/group; B total n=9 mice/group. Significance represents p<0.05. Data were analyzed using one-way ANOVA with Tukey correction and represented as mean±SEM.

FIGS. 30A-30C illustrate In vitro activation of T cells does not induce Zbtb20 expression. FIG. 30A. Expression of Zbtb20-GFP in spleen CD4⁺ and CD8⁺ T cells from ZEG20;FoxP3-RFP mice prior to activation. FIG. 30B. ZEG20 spleen cells were activated with 2.5 μg/mL anti-CD3 antibody for 4 days then analyzed by FACS to determine if Zbtb20-GFP expression in CD4⁺ and CD8⁺ T cells was induced. The cells were rested for a total of 6 days and then reactivated with anti-CD3/anti-CD28 beads (Miltenyi) for 4 more days and Zbtb20-GFP was assessed by FACS. WT spleen cells were also activated and used as controls for FACS analysis. FIG. 30C. GFP⁻ CD4⁺ T cells collected from ZEG20 mice were activated with 5 μg/ml plate-bound anti-CD3 (2C11) and 5 μg/ml soluble anti-CD28 and cultured for 72 h in the presence of TGFβ, IL-6 or both. At least two independent experiments were performed. A,B total n=4 mice/group; C total n=3 mice/group.

FIGS. 31A-31B illustrate that Zbtb20 is not induced in non-expressing T cells in vivo. FIG. 31A. GFP⁻, CD62L^{hi} CD4⁺ CD45.2⁺ T cells collected from spleens of ZEG20 mice were i.p. injected into the non-transgenic B6.SJL (CD45.1⁺) mice. The expression of Zbtb20-GFP in donor cells in Payer's patches of the recipient mice was assessed after 2 weeks by FACS. FIG. 31B. Sorted GFP⁻ CD4SP thymocytes from ZEG20;FoxP3-RFP (CD45.1⁺/CD45.2⁺) mice were i.p. injected into B6.SJL (CD45.1⁺) mice; the recipient mice received DSS to induce colitis for 5 days followed by a 4-day recovery period. The expression of Zbtb20-GFP in T cells from the spleen, small intestine LP, colon LP, Peyer's Patches, small intestine IEL and colon IEL were analyzed by FACS to determine if Zbtb20 had been induced 10 days post-injection. The GFP expression in non-transgenic recipient mice was used to assess the background level. At least two independent experiments were performed. A total n=3 mice/group; B total n=6 mice/group.

FIGS. 32A-32C illustrate TIL from MB49 grown in Zbtb20 reporter mice. (FIG. 32A) Total TIL population. (FIG. 32B) Total CD4+ T cell population. (FIG. 32C) Zbtb20 expression in CD4+CD25+ Tregs.

FIG. 33 illustrates a comparison of tumor growth in wild type (black line; N=24) or cKO mice (purple line; N=21). Data are from 4 independent experiments.

FIG. 34 illustrates a flow cytometry stain for PD-1 expression on spleen Zbtb20+ Tregs as compared to non-Zbtb20 Tregs.

FIGS. 35A-35B illustrate the impact of Zbtb20 depletion on anti-PD1 treatment. (FIG. 35A) Wild type or age/sex matched (FIG. 35B) cKO mice were implanted with DM4-BRaf^{V600E} melanoma cells. Mice received injections of anti-PD1 or an isotype control mAb on the days circled in red (FIG. 35A) black=iso; Grey=PD1 (FIG. 35B) Dark=iso; Grey=PD1. N=6 mice per group.

FIG. 36 illustrates FACS analysis of T cells isolated from tumors. The TIL T cells from WT mice treated with anti-PD1 nearly all have an activated CD44 high phenotype, whereas the TIL T cells from cKO mice are mostly not activated (CD44 low).

FIG. 37 illustrates that the number of F4/80+ macrophages is increased in the intestine of Zbtb20 KO mice following DSS treatment.

### DETAILED DESCRIPTION

### Definitions

Unless defined otherwise, all technical and scientific terms used herein have the same meaning as commonly understood by one of ordinary skill in the art to which the disclosure pertains. Although any methods and materials similar or equivalent to those described herein can be used in the practice for testing of the present disclosure, the preferred materials and methods are described herein. In describing and claiming the present disclosure, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20% or ±10%, more preferably ±5%, even more preferably ±1%, and still more preferably ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, to “alleviate” a disease means reducing the severity of one or more symptoms of the disease.

“Allogeneic” refers to a graft derived from a different animal of the same species.

“Alloantigen” refers to an antigen present only in some individuals of a species and capable of inducing the production of an alloantibody by individuals which lack it.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present disclosure may exist in a variety of forms including, for example, polyclonal antibodies, monoclonal antibodies, Fv, Fab and F(ab)₂, as well as single chain antibodies (scFv) and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. α and β light chains refer to the two major antibody light chain isotypes.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it is later to be re-introduced into the individual.

“Allogeneic” refers to any material derived from a different animal of the same species.

As used herein, the phrase “cancer treatment response” refers to any physiological indication that the cancer, and/or symptom thereof, is being treated and/or ameliorated. Non-limiting examples of such cancer treatment responses include shrinkage and/or disappearance of a tumor, reduction in tumor growth rate, reduction and/or inhibition of metastatic growth(s), reduction in tumor angiogenesis, increase in patient's survival, increase in overall patient's energy and/or stamina, reduction in patient's nausea, and so forth.

“Co-stimulatory ligand,” as the term is used herein, includes a molecule on an antigen presenting cell (e.g., an APC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

The term “regulatory T cell” or “Treg”, as used herein, refers to a specialized population of primarily CD4+ T cells which function as negative regulators of T cell responses. Regulatory T cells may be thymically derived or arise in the periphery as a consequence of certain inflammatory and non-inflammatory conditions. Tregs are essential for maintaining immune tolerance to self and immune homeostasis, and their dysregulation can lead to inappropriate inflammatory responses or over-regulated and inefficacious responses.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune suppression or tolerance compared to the immune response detected in the absence of the composition of the disclosure. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly about 10 amino acids and/or sugars in size. Preferably, the epitope is about 4-18 amino acids, more preferably about 5-16 amino acids, and even more most preferably 6-14 amino acids, more preferably about 7-12, and most preferably about 8-10 amino acids. One skilled in the art understands that generally the overall three-dimensional structure, rather than the specific linear sequence of the molecule, is the main criterion of antigenic specificity and therefore distinguishes one epitope from another. Based on the present disclosure, a peptide of the present disclosure can be an epitope.

As used herein, the term “exogenous” refers to any material introduced from or produced outside an organism, cell, tissue or system.

The term “expand” as used herein refers to increasing in number, as in an increase in the number of T cells. In certain embodiments, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “immune response” as used herein is defined as a cellular response to an antigen that occurs when lymphocytes identify antigenic molecules as foreign and induce the formation of antibodies and/or activate lymphocytes to remove the antigen.

The term “immunostimulatory” is used herein to refer to increasing overall immune response.

The term “immunosuppressive” is used herein to refer to reducing overall immune response.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

By the term “modified” as used herein, is meant a changed state or structure of a molecule or cell of the disclosure. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “self-antigen” as used herein is defined as an antigen that is expressed by a host cell or tissue. Self-antigens may be tumor antigens, but in certain embodiments, are expressed in both normal and tumor cells. A skilled artisan would readily understand that a self-antigen may be overexpressed in a cell.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

A “target site” or “target sequence” refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (a) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

“Transplant” refers to a biocompatible lattice or a donor tissue, organ or cell, to be transplanted. An example of a transplant may include but is not limited to skin cells or tissue, bone marrow, and solid organs such as heart, pancreas, kidney, lung and liver. A transplant can also refer to any material that is to be administered to a host. For example, a transplant can refer to a nucleic acid or a protein.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

“Xenogeneic” refers to any material derived from an animal of a different species.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The current disclosure is based in certain aspects on the surprising finding that a subset of CD4⁺ regulatory T cells (Tregs) and CD4+ T cells express the transcription factor ZBTB20. In these cells, ZBTB20 acts as a regulator of cell function by inducing certain phenotypic and functional changes in the cells, including the enhanced expression of IL-10, a cytokine associated with regulation of immune responses. T cells expressing high levels of ZBTB20 have been found to be key regulators of immune homeostasis in gastrointestinal mucosa, and perturbation of normal expression leads to inflammatory diseases of the gut that resemble human inflammatory bowel diseases. As such, in certain aspects, the current disclosure includes cells which have been engineered to overexpress ZBTB20 protein, which can be used to dampen inappropriate inflammatory immune responses in subjects. In certain aspects of the disclosure, therapeutic compositions comprising the ZBTB20 overexpression cells are also contemplated. In certain aspects, the disclosure also includes methods for treating inflammatory diseases in subject using cells engineered to express ZBTB20, as well as methods of determining a subject's risk of developing an inflammatory disease comprising assessing the level of ZBTB20 expression in a tissue sample from the subject.

The current disclosure is further based in certain aspects on the surprising finding that the frequency of Zbtb20 expressing T cells in a tumor may be predicative of successful treatment with anti-PD1 and that tumor growth is accelerated in Zbtb20 knockout mice. Further, in certain embodiments, introduction of T cells transduced with ZBTB20 is a cellular therapy to improve anti-PD1 induced responses to tumors.

### Regulatory T Cells and IL-10:

The majority of Tregs are CD4+ and express the lineage defining transcription factor, FoxP3 (forkhead box protein 3) that is responsible for most of the characteristic features of these cells, such as the expression of CD25 (IL2RA—Interleukin 2 Receptor Subunit Alpha) and CTLA-4 (cytotoxic T-lymphocyte antigen 4). Phenotypic diversity and tissue specialization among Tregs imply the existence of functional subsets. For example, Foxp3+ Tregs that express the IL-33 receptor ST2 and the transcription factors GATA3 and Helios, in response to the danger signals during inflammation produce high levels of amphiregulin (Areg), which mediates tissue repair. Another subset of Foxp3+ Tregs, defined by expression of RORγt, ICOS, CTLA4, and the nucleotidases CD39 and CD73, has enhanced suppressive capacity during the T-cell-mediated intestinal inflammation. Thus, the tissue-specific milieu and the presence of different transcription factors appear to contribute to the functional diversity of Tregs. In certain embodiments, the current disclosure includes isolated T cells which have been modified to express high levels of the ZBTB20 transcription factor. In certain embodiments, the isolated T cells are regulatory T cells (Tregs).

Regulatory T cells (Tregs) use various mechanisms to balance the immune response, but the secretion of Interleukin-10 (IL-10) is of particular importance for modulating intestinal homeostasis. IL-10 has pleiotropic effects, impacting the proliferation and/or differentiation of numerous cell types including dendritic cells (DCs), B and T cells, Tregs, and natural killer (NK) cells. It has also been shown to reduce the expression of MHC II, co-stimulatory molecules (CD80, CD86), and cytokine production. Although IL-10 can be expressed by many types of immune cells, CD4+ T cells are its main source in the gastrointestinal tract. Interestingly though, FoxP3 is not directly responsible for the production of IL-10 by Tregs as the IL10 locus lacks the binding site for FoxP3. However, FoxP3+CD4+ T cells (Tregs) are the primary source of IL-10 in the colon, whereas FoxP3-CD4+ T cells (Type 1 regulatory T cells; Tr1) represent a major producer of the cytokine in the small intestine. Naïve CD4+ T cells do not express IL-10 since the chromatin in the vicinity of the Il10 gene is closed. On the other hand, terminally differentiated, mature Tregs have the Il10 locus in a transcriptionally competent state allowing IL-10 production. The expression of IL-10 is tightly regulated through chromatin structure and histone modification, but also DNA methylation and active transcription factors. Therefore, IL-10 is an effector cytokine that is produced as a result of activation and differentiation of T cells, including Tregs. In certain embodiments of the current disclosure, the expression of ZBTB20 by the isolated cell results in enhanced IL-10 production as compared to unmodified cells.

### ZBTB20:

The BTB-ZF (broad-complex, tramtrack and bric-à-brac—zinc finger) genes are a family of 49 transcription factors defined by the presence of an N-terminal BTB domain that is involved in protein-protein interactions, and DNA binding C-terminal Krüppel-type zinc fingers. Recent studies have shown that BTB-ZF transcription factors control the commitment of developing lymphocytes to specific lineages and control functions of mature cells. In the studies of the current disclosure, expression of the BTB-ZF transcription factor ZBTB20 defines distinct subtypes of CD4⁺ T cells and FoxP3⁺ Tregs. Both T cell types are found in the thymus and spleen and are substantially enriched in the intestine. ZBTB20 expressing T cells have an activated phenotype (CD62L^{lo}, CD44^{hi}). Importantly, ZBTB20-expressing T cells constitutively express IL-10 message and rapidly secrete IL-10 upon primary stimulation. Interestingly, these phenotypic and functional characteristics were observed not only in mature T cells but also in Zbtb20 expressing thymocytes. Therefore, similar to NKT cells, ZBTB20 expressing T cells have “innate-like” features that are acquired during development. In certain embodiments of the current disclosure, isolated cells are modified to comprise nucleic acid vectors comprising a gene encoding ZBTB20 protein. In certain embodiments, the expression of ZBTB20 resulting from the vector alters the function of the cell such that IL-10 production is enhanced relative to cells not comprising the nucleic acid vector.

### Modified Immune Cells

The present disclosure provides modified cells or precursors thereof (e.g., T cells) comprising a nucleic acid vector encoding ZBTB20. The disclosure also includes modified cells comprising any of the nucleic acids disclosed herein or any of the vectors disclosed herein.

In certain embodiments, the modified cell is a modified immune cell. In certain embodiments, the modified cell is a modified T cell. It is contemplated that both CD4+ and CD8+ T cells could be modified by introduction of the ZBTB20-encoding vectors of the invention. In certain embodiments, the T cell is a regulatory T cell. Regulatory T cell populations can include both CD4+ and CD8+ regulatory T cells. In certain embodiments, the regulatory T cell is a CD4+ regulatory T cell (Treg). In certain embodiments, the modified cell is an autologous cell. In certain embodiments, the modified cell is an autologous cell obtained from a mammalian subject. In certain embodiments the subject is mouse. In certain embodiments, the subject is human.

Methods of Treatment, Amelioration, and/or Prevention

The modified cells (e.g., ZBTB20-expressing T cells) described herein, may be included in a composition for immunotherapy. The composition may include a pharmaceutical composition and further include a pharmaceutically acceptable carrier. A therapeutically effective amount of the pharmaceutical composition comprising the modified cells may be administered.

In one aspect, the disclosure includes a method for treating, ameliorating, and/or preventing an inflammatory disease in a subject in need thereof, the method comprising administering to the subject an effective amount of the modified cells of the disclosure, thereby treating, ameliorating, and/or preventing the inflammatory disease. In another aspect, the disclosure includes a method of treating, ameliorating, and/or preventing an inflammatory disease in a subject in need thereof, the method comprising: isolating a cell from the subject, contacting the cell with a nucleic acid vector encoding ZBTB20 such that expression of ZBTB20 protein is elevated as compared to uncontacted cells, thereby inducing an anti-inflammatory function in the cell, and administering the contacted cell to the subject thereby treating, ameliorating, and/or preventing the inflammatory disease. In certain embodiments, the inflammatory disease is a gastrointestinal inflammatory disease. In certain embodiments, it is contemplated that the gastrointestinal inflammatory disease is selected from a group comprising, but not limited to, inflammatory bowel disease (IBD), Crohn's disease, and ulcerative colitis. In certain embodiments, the modified cells are autologous to the subject. In certain embodiments, the modified cells are heterologous to the subject.

In one aspect, the disclosure includes a method for adoptive cell transfer therapy comprising administering to a subject in need thereof a modified T cell of the present disclosure. In another aspect, the disclosure includes a method of treating a disease or condition in a subject comprising administering to a subject in need thereof a population of modified T cells. In certain embodiments, the disease to be treated is an inflammatory disease. In certain embodiments, the inflammatory disease is a gastrointestinal inflammatory disease. Any number of gastrointestinal inflammatory diseases are contemplated to be treated by the modified cells of the disclosure, including but not limited to inflammatory bowel disease (IBD), Crohn's disease, and ulcerative colitis or any combination thereof.

In certain embodiments, the cells of the disclosure can be used to treat an inflammatory disease resulting from allogeneic transplant. In certain embodiments, the inflammatory disease resulting from allogenic transplant is graft versus host disease (GVHD). Allogenic bone marrow transplant or allogenic stem cell transplant is a treatment strategy used in a number of cancers in which donor blood stem cells are implanted to replace a patient's diseased or damaged bone marrow cells in a number of cancer and non-cancer diseases including but not limited to acute leukemia, adrenoleukodystrophy, aplastic anemia, bone marrow failure syndromes, chronic leukemia, hemoglobinopathies, Hodgkin's lymphoma, immune deficiencies, inborn errors of metabolism, multiple myeloma, myelodysplastic syndromes, neuroblastoma, non-Hodgkin's lymphoma, plasma cell disorders, POEMS syndrome, and primary amyloidosis. Because the transplanted bone marrow cells are genetically distinct from the tissues of the recipient, T cells derived from the transplanted tissue (the graft) can often recognize the recipient's tissues as foreign (the host) and attack them. The gastrointestinal system is one of the most common tissue sites for the manifestation of GVHD, and severe reactions are associated with poor prognosis. In certain embodiments the cells of the disclosure are used to treat or ameliorate GVHD inflammatory responses in specific tissue sites, including the gastrointestinal tract, among others.

In one aspect, the disclosure includes a method of immunotherapy for cancer for use in a subject in need thereof. In certain embodiments, the method comprises isolating an immune cell from the subject. In certain embodiments, the method comprises contacting the immune cell with a nucleic acid vector encoding ZBTB20 such that expression of ZBTB20 protein is elevated in the immune cell as compared to uncontacted immune cells. In certain embodiments, the method comprises administering the contacted immune cell to the subject thereby treating, ameliorating, and/or preventing the cancer. In certain embodiments, the method further comprises administering to the subject an anti-PD-1 therapy. Administration of the anti-PD-1 therapy can occur prior to, following, or concurrent with administration of the ZBTB20 modified T cells. In this way, elevated expression of ZBTB20 potentiates the immunotherapeutic effects of the anti-PD-1 therapy and results in a more efficacious anti-tumor immune response. In certain embodiments, the anti-PD-1 therapy is an antibody. In certain embodiments, the anti-PD-1 therapy is an antibody specific for PD-1. In certain embodiments, the anti-PD-1 therapy is an antibody specific for PD-L1. Antibody-based inhibitors of the PD-1/PD-L1 signaling axis are commonly known in the art and have become a mainstay or front-line treatment for certain cancers. It is contemplated that any available anti-PD-1 therapy (such as an anti-PD-1/PD-L1 antibody) appropriate for clinical use could be used in the method of the invention including but not limited to pembrolizumab, nivolumab, cemiplimab, atezolizumab, avelumab, dostarlimab, and durvalumab or any combination thereof, among others. It is also contemplated that the anti-PD-1 therapy (such as anti-PD-1/PD-L1 antibody treatment) could be combined with any other immunotherapy (such as anti-CTLA-4, anti-TIGIT, anti-LAG-3, anti-Tim-3, and the like) or chemotherapy. In certain embodiments, the immune cell is a T cell. It is contemplated that modification which increases ZBTB20 expression can benefit effector T cells of multiple lineages in order to enhance anti-tumor immune responses. Thus, in certain embodiments, the T cell is a CD4+ T cell, a CD8+ T cell, or a mixture of CD4+ and CD8+ T cells.

In certain embodiment, the enhanced expression of ZBTB20 in immune cells potentiates effectiveness of anti-PD-1 therapy against cancers. Thus, in certain embodiments, the cancer is a solid cancer or a blood cancer. Any cancer which could be treated with an anti-PD-1 therapy could be treated with the methods of the invention. Non-limiting examples of such cancers include melanoma, head and neck cancer, non-small cell lung cancer, bladder cancer, and any microsatellite unstable cancers which correlate to favorable targeting by immunotherapies.

In one aspect, the disclosure includes a method of determining whether a subject is a candidate for cancer treatment with anti-PD-1 therapy. In certain embodiments, the method comprises obtaining a tumor sample from the subject. In certain embodiments, the method further comprises assessing the level of ZBTB20 expression in a cell of the tumor sample. In certain embodiments, the method further comprises comparing the level of ZBTB20 expression to a baseline expression level established from tumors which were successfully treated with anti-PD-1 therapy. In certain embodiments, ZBTB20 levels in the tumor sample being lower than the baseline expression level represents a decreased likelihood of successful anti-PD-1 therapy. In certain embodiments, wherein ZBTB20 levels in the tumor sample are lower than the baseline expression level, the subject is not selected for anti-PD-1 therapy. In certain embodiments, ZBTB20 levels are assessed in tumor-resident T cells of both CD4+ and CD8+ lineages. Studies of the present disclosure have demonstrated that T cells with low or no expression of ZBTB20 do not benefit from treatment with PD-1/PD-L1 inhibitors when used in the setting of tumor immunotherapy. Thus the screening methods of the present disclosure can be used to assess the suitability for solid tumors for treatment with PD-1/PD-L1 inhibitors. Non-limiting examples of such tumors include melanoma, head and neck cancer, non-small cell lung cancer, bladder cancer among others. In certain embodiments, the anti-PD-1 therapy is an antibody blockade therapy. In certain embodiments, the antibody targets PD-1 or PD-L1.

Methods for administration of modified cells for adoptive cell therapy are known in the art and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive T cell therapy is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and modification are administered to the same subject.

In some embodiments, the cell therapy, e.g., adoptive T cell therapy, is carried out by allogeneic or heterologous transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The modified immune cells of the present disclosure can be administered to an animal, preferably a mammal, even more preferably a human, to treat an inflammatory disease (e.g. inflammatory bowel disease). In addition, the cells of the present disclosure can be used for the treatment of any condition related to an inflammatory disease, especially a gastrointestinal inflammatory disease, where it is desirable to treat or alleviate the disease. The types of inflammatory diseases to be treated with the modified cells or pharmaceutical compositions of the disclosure include inflammatory bowel disease (IBD), Crohn's disease, and ulcerative colitis, and the like. In certain embodiments, the inflammatory disease is inflammatory bowel disease (IBD). In certain embodiments, the inflammatory disease is a Crohn's disease. In certain embodiments, the inflammatory disease is ulcerative colitis.

The cells of the disclosure to be administered may be autologous, with respect to the subject undergoing therapy.

The administration of the cells of the disclosure may be carried out in any convenient manner known to those of skill in the art. The cells of the present disclosure may be administered to a subject by aerosol inhalation, injection, ingestion, transfusion, implantation or transplantation. The compositions described herein may be administered to a patient transarterially, subcutaneously, intradermally, intranodally, intramedullary, intramuscularly, by intravenous (i.v.) injection, or intraperitoneally. In other instances, the cells of the disclosure are injected directly into a site of inflammation in the subject, a local disease site in the subject, a lymph node, an organ, a tumor, and the like.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD4⁺ regulatory T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4+ to ZBTB20+ ratio), e.g., within a certain tolerated difference or error of such a ratio.

In certain embodiments, the cells, or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 1×10⁵ cells/kg to about 1×10¹¹ cells/kg 10⁴ and at or about 10¹¹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at or about 1×10⁵ T cells/kg, 1.5×10⁵ T cells/kg, 2×10⁵ T cells/kg, or 1×10⁶ T cells/kg body weight. In other exemplary embodiments, a suitable dosage range of modified cells for use in a method of the present disclosure includes, without limitation, from about 1×10⁵ cells/kg to about 1×10⁶ cells/kg, from about 1×10⁶ cells/kg to about 1×10⁷ cells/kg, from about 1×10⁷ cells/kg about 1×10⁸ cells/kg, from about 1×10⁸ cells/kg about 1×10⁹ cells/kg, from about 1×10⁹ cells/kg about 1×10¹⁰ cells/kg, from about 1×10¹⁰ cells/kg about 1×10¹¹ cells/kg. In an exemplary embodiment, a suitable dosage for use in a method of the present disclosure is about 1×10⁸ cells/kg. In an exemplary embodiment, a suitable dosage for use in a method of the present disclosure is about 1×10⁷ cells/kg. In other embodiments, a suitable dosage is from about 1×10⁷ total cells to about 5×10⁷ total cells. In some embodiments, a suitable dosage is from about 1×10⁸ total cells to about 5×10⁸ total cells. In some embodiments, a suitable dosage is from about 1.4×10⁷ total cells to about 1.1×10⁹ total cells. In an exemplary embodiment, a suitable dosage for use in a method of the present disclosure is about 7×10⁹ total cells.

In some embodiments, the cells are administered at or within a certain range of error of between at or about 10⁴ and at or about 10⁹ CD4⁺ and/or CD8⁺ cells/kilograms (kg) body weight, such as between 10⁵ and 10⁶ CD4⁺ and/or CD8⁺ cells/kg body weight, for example, at or about 1×10⁵ CD4⁺ and/or CD8⁺ cells/kg, 1.5×10⁵ CD4⁺ and/or CD8⁺ cells/kg, 2×10⁵ CD4⁺ and/or CD8⁺ cells/kg, or 1×10⁶ CD4⁺ and/or CD8⁺ cells/kg body weight. In some embodiments, the cells are administered at or within a certain range of error of, greater than, and/or at least about 1×10⁶, about 2.5×10⁶, about 5×10⁶, about 7.5×10⁶, or about 9×10⁶ CD4⁺ cells, and/or at least about 1×10⁶, about 2.5×10⁶, about 5×10⁶, about 7.5×10⁶, or about 9×10⁶ CD8⁺ cells, and/or at least about 1×10⁶, about 2.5×10⁶, about 5×10⁶, about 7.5×10⁶, or about 9×10⁶ T cells. In some embodiments, the cells are administered at or within a certain range of error of between about 10⁸ and 10¹² or between about 10¹⁰ and 10¹¹ T cells, between about 10⁸ and 10¹² or between about 10¹⁰ and 10¹¹ CD4⁺ cells, and/or between about 10⁸ and 10¹² or between about 10¹⁰ and 10¹¹ CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios, for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In some embodiments, a dose of modified cells is administered to a subject in need thereof, in a single dose or multiple doses. In some embodiments, a dose of modified cells is administered in multiple doses, e.g., once a week or every 7 days, once every 2 weeks or every 14 days, once every 3 weeks or every 21 days, once every 4 weeks or every 28 days. In an exemplary embodiment, a single dose of modified cells is administered to a subject in need thereof. In an exemplary embodiment, a single dose of modified cells is administered to a subject in need thereof by rapid intravenous infusion.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

Cells of the disclosure can be administered in dosages and routes and at times to be determined in appropriate pre-clinical and clinical experimentation and trials. Cell compositions may be administered multiple times at dosages within these ranges. Administration of the cells of the disclosure may be combined with other methods useful to treat the desired disease or condition as determined by those of skill in the art.

### Vectors

A vector may be used to introduce the gene encoding ZBTB20 into a T cell as described elsewhere herein. In certain aspects, the disclosure includes vectors comprising nucleic acid sequences encoding ZBTB20. The vector can comprise a plasmid vector, viral vector, retrotransposon (e.g. piggyback, sleeping beauty), site directed insertion vector (e.g. CRISPR, Zn finger nucleases, TALEN), suicide expression vector, lentiviral vector, RNA vector, or other known vector in the art.

The production of any of the molecules described herein can be verified by sequencing. Expression of the full length proteins may be verified using immunoblot, immunohistochemistry, flow cytometry or other technology well known and available in the art.

The present disclosure also provides a vector in which DNA of the present disclosure is inserted. Vectors, including those derived from retroviruses such as lentivirus, are suitable tools to achieve long-term gene transfer since they allow long-term, stable integration of a transgene and its propagation in daughter cells. Lentiviral vectors have the added advantage over vectors derived from onco-retroviruses, such as murine leukemia viruses, in that they can transduce non-proliferating cells, such as hepatocytes. They also have the added advantage of resulting in low immunogenicity in the subject into which they are introduced.

The expression of natural or synthetic nucleic acids is typically achieved by operably linking a nucleic acid or portions thereof to a promoter, and incorporating the construct into an expression vector. The vector is one generally capable of replication in a mammalian cell, and/or also capable of integration into the cellular genome of the mammal. Typical vectors contain transcription and translation terminators, initiation sequences, and promoters useful for regulation of the expression of the desired nucleic acid sequence.

The nucleic acid can be cloned into any number of different types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

The expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, the EF-1 alpha promoter, as well as human gene promoters such as, but not limited to, the actin promoter, the myosin promoter, the hemoglobin promoter, and the creatine kinase promoter. Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess expression of a polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

### Introduction of Nucleic Acids

Methods of introducing nucleic acids into a cell include physical, biological and chemical methods. Physical methods for introducing a polynucleotide, such as RNA or DNA, into a host cell include calcium phosphate precipitation, lipofection, particle bombardment, microinjection, electroporation, and the like. RNA or DNA can be introduced into target cells using commercially available methods which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany). RNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8):861-70 (2001).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present disclosure, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

Moreover, the nucleic acids may be introduced by any means, such as transducing the expanded T cells, transfecting the expanded T cells, and electroporating the expanded T cells. One nucleic acid may be introduced by one method and another nucleic acid may be introduced into the T cell by a different method.

### Sources of Immune Cells

In certain embodiments, a source of immune cells, including T cells, is obtained from a subject. Non-limiting examples of subjects include humans, dogs, cats, mice, rats, and transgenic species thereof. In some preferred embodiments, the subject is a human. In some preferred embodiments, the subject is a mouse. T cells can be obtained from a number of sources, including peripheral blood mononuclear cells, bone marrow, lymph node tissue, spleen tissue, umbilical cord, and tumors. In certain embodiments, any number of T cell lines available in the art, may be used. In certain embodiments, T cells can be obtained from a unit of blood collected from a subject using any number of techniques known to the skilled artisan, such as Ficoll separation. In certain embodiments, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood by lysing the red blood cells and depleting the monocytes, for example, by centrifugation through a PERCOLL™ gradient. Alternatively, T cells can be isolated from umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19 and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, In certain embodiments, a concentration of 2 billion cells/ml is used. In certain embodiments, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

Immune cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain embodiments, the population of immune cells is comprised within cells such as peripheral blood mononuclear cells, cord blood cells, a purified population of T cells, and a T cell line. In another embodiment, peripheral blood mononuclear cells comprise the population of immune cells. In yet another embodiment, purified T cells comprise the population of immune cells.

In certain embodiments, T regulatory cells (Tregs) can be isolated from a sample. The sample can include, but is not limited to, umbilical cord blood or peripheral blood. In certain embodiments, the Tregs are isolated by flow-cytometry sorting. The sample can be enriched for Tregs prior to isolation by any means known in the art. The isolated Tregs can be cryopreserved, and/or expanded prior to use. Methods for isolating Tregs are described in U.S. Pat. Nos. 7,754,482, 8,722,400, and 9,555,105, and U.S. patent application Ser. No. 13/639,927, contents of which are incorporated herein in their entirety.

### Expansion of T Cells

In certain embodiments, the T cells disclosed herein can be multiplied by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In certain embodiments, the T cells expand in the range of about 20 fold to about 50 fold.

Following culturing, the T cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. The T cell medium may be replaced during the culture of the T cells at any time. Preferably, the T cell medium is replaced about every 2 to 3 days. The T cells are then harvested from the culture apparatus whereupon the T cells can be used immediately or cryopreserved to be stored for use at a later time. In certain embodiments, the disclosure includes cryopreserving the expanded T cells. The cryopreserved T cells are thawed prior to introducing nucleic acids into the T cell.

In another embodiment, the method comprises isolating T cells and expanding the T cells. In another embodiment, the disclosure further comprises cryopreserving the T cells prior to expansion. In yet another embodiment, the cryopreserved T cells are thawed for electroporation with the nucleic acid encoding the ZBTB20 protein.

Another procedure for ex vivo expansion cells is described in U.S. Pat. No. 5,199,942 (incorporated herein by reference). Expansion, such as described in U.S. Pat. No. 5,199,942 can be an alternative or in addition to other methods of expansion described herein. Briefly, ex vivo culture and expansion of T cells comprises the addition to the cellular growth factors, such as those described in U.S. Pat. No. 5,199,942, or other factors, such as flt3-L, IL-1, IL-3 and c-kit ligand. In certain embodiments, expanding the T cells comprises culturing the T cells with a factor selected from the group consisting of flt3-L, IL-1, IL-3 and c-kit ligand.

The culturing step as described herein (contact with agents as described herein or after electroporation) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but is not limited to the seeding density, substrate, medium, and time between passaging.

In certain embodiments, the cells may be cultured for several hours (about 3 hours) to about 14 days or any hourly integer value in between. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-α. or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

The medium used to culture the T cells may include an agent that can co-stimulate the T cells. For example, an agent that can stimulate CD3 is an antibody to CD3, and an agent that can stimulate CD28 is an antibody to CD28. This is because, as demonstrated by the data disclosed herein, a cell isolated by the methods disclosed herein can be expanded approximately 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater. In certain embodiments, the T cells expand in the range of about 20 fold to about 50 fold, or more by culturing the electroporated population. In certain embodiments, human T regulatory cells are expanded via anti-CD3 antibody coated KT64.86 artificial antigen presenting cells (aAPCs). Methods for expanding and activating T cells can be found in U.S. Pat. Nos. 7,754,482, 8,722,400, and 9,555,105, contents of which are incorporated herein in their entirety.

In certain embodiments, the method of expanding the T cells can further comprise isolating the expanded T cells for further applications. In another embodiment, the method of expanding can further comprise a subsequent electroporation of the expanded T cells followed by culturing. The subsequent electroporation may include introducing a nucleic acid encoding an agent, such as a transducing the expanded T cells, transfecting the expanded T cells, or electroporating the expanded T cells with a nucleic acid, into the expanded population of T cells, wherein the agent further stimulates the T cell. The agent may stimulate the T cells, such as by stimulating further expansion, effector function, or another T cell function.

### Pharmaceutical Compositions

Pharmaceutical compositions of the present disclosure may comprise the modified immune cell as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are preferably formulated for intravenous administration.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

The cells of the disclosure to be administered may be autologous, allogeneic or xenogeneic with respect to the subject undergoing therapy.

Cells of the disclosure can be administered in dosages and routes and at times to be determined in appropriate pre-clinical and clinical experimentation and trials. Cell compositions may be administered multiple times at dosages within these ranges. Administration of the cells of the disclosure may be combined with other methods useful to treat the desired disease or condition as determined by those of skill in the art.

It can generally be stated that a pharmaceutical composition comprising the modified T cells described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

The administration of the modified immune cells of the disclosure may be carried out in any convenient manner known to those of skill in the art. The cells of the present disclosure may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the disclosure are injected directly into a site of inflammation in the subject, a local disease site in the subject, a lymph node, an organ, a tumor, and the like.

It should be understood that the method and compositions that would be useful in the present disclosure are not limited to the particular formulations set forth in the examples. The following examples are put forth so as to provide those of ordinary skill in the art with a complete disclosure and description of how to make and use the cells, expansion and culture methods, and therapeutic methods of the disclosure, and are not intended to limit the scope of what the inventors regard as their disclosure.

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Current Protocols in Immunology” (Coligan, 2002). These techniques are applicable to the production of the polynucleotides and polypeptides of the disclosure, and, as such, may be considered in making and practicing the disclosure. Particularly useful techniques for particular embodiments will be discussed in the sections that follow.

### EXPERIMENTAL EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present disclosure and practice the claimed methods. The following working examples therefore, specifically point out the preferred embodiments of the present disclosure, and are not to be construed as limiting in any way the remainder of the disclosure.

The Materials and Methods used in the performance of the experiments disclosed herein are now described.

Mice. Zbtb20 enhanced green fluorescent protein reporter mice (ZEG20) were made with an engineered BAC obtained from the Gene Expression Nervous System Atlas (GENSAT) Purified BAC DNA was microinjected into fertilized C57BL/6 eggs by MSKCC's Mouse Genetics Core Facility. The resulting founders were backcrossed to C57BL/6 mice and screened for GFP expression by FACS. Multiple founders showed similar expression and one was selected for further study. The CD4-Cre, Foxp3^{ires-mrfp} (FIR), IL-10^{ires-GFP} (tiger), RAG1^{-/-}, C57Bl/6, CD4-Cre, and SJL mice were purchased from The Jackson Laboratory (Bar Harbor, ME). Zbtb20^{fox/flox} mice were generated at Dr. Lynn Corcoran laboratory (The Walter and Eliza Hall Institute) using conventional methods of homologous recombination in embryonic stem (ES) cells derived from C57BL/6 mice. The targeting vector contained 5.4 kb (5′) and 4.2 kb (3′) of homologous Zbtb20 genomic sequence flanking a 1605 bp central protein-coding exon, which was flanked by loxP sites. In these mice, Cre-mediated recombinase deletes the exon 14 (Transcript: Zbtb20-204 ENSMUST00000114694.8), resulting in a Zbtb20 protein lacking 535 central residues, including the BTB/POZ domain and the first Zn finger.

Unless otherwise mentioned, experiments were done using 8-12-week old mice (age- and sex-matched). Both sexes were included to account for possible biological variability. Mice were co-housed at least 4 weeks before each experiment to account for potential differences in the microbiome. Mice were euthanized by carbon dioxide asphyxiation, followed by cervical dislocation. All mouse strains were bred and maintained in the CHINJ animal facility. Animal care and experimental procedures were carried out following the guidelines of the Institutional Animal Care and Use Committee of Rutgers University and the National Institutes of Health Guide for the Care and Use of Laboratory Animals.

Cell isolation. Single cell suspensions were made by dissociation of tissues between glass slides. The isolated cells were filtered through a 40-μm mesh followed by treatment with RBC Lysing Buffer (Sigma-Aldrich). The PPs were collected from the small intestine, dissociated between glass slides, and filtered through a 40-μm mesh. To collect the IEL, the small intestines and colons were longitudinally opened, rinsed in Ca²⁺- and Mg²⁺-free HBSS (Sigma-Aldrich), and incubated on a shaker (at 250 rpm), in HBSS with 5% (wt/vol) Heat Inactivated Fetal Bovine Serum (HI FBS) (Gibco) and 2 mM EDTA for 1 h at 37° C. Supernatants were filtered through a 100-μm mesh and applied to columns packed with nylon wool to remove debris. The LP leukocytes were isolated by two 20 min consecutive digestions with 100 U/mL and 200 U/mL of Collagenase type IV (Worthington) in RPMI (Gibco) with 5% (wt/vol) HI FBS.

Flow Cytometry and cell sorting. Surface staining of the cells was performed for 30 min at 4° C. in FACS buffer (PBS with 1% HI FBS) after blocking (15 min) with 2% normal mouse serum, 0.1% anti-Fcγ Ab, and 0.1 mg/mL streptavidin. Intracellular staining for transcription factors was done at room temperature using the Foxp3/Transcription Factor Staining Buffer Set (eBioscience). The following antibodies were used in this study: anti-CD4 (RM4-5)(GK1.5), anti-CD62L (MEL-14), and anti-Zbtb20 (4A3) (BD Bioscience). Anti-CD44 (IM7), anti-CD45.2 (104), anti-CD45.1(A20), anti-CD3 (500.A2), anti-CD25 (PC61)(PC61.5), anti-CD8 (53-6.7), anti-ICOS (15F9), anti-GITR (DTA-1), anti-FoxP3 (FJK-16s), anti-TIGIT (GIGD7) and Neuropilin-1 (3DS304M) (eBioscience). Anti-MHC II (212.A1) was generated by the MSKCC Ab Core Facility. Dead cells were excluded when possible, by DAPI staining, and doublet events were eliminated by comparing forward scatter width to forward scatter height and side scatter width to side scatter height. The data were acquired on an LSRII cytometer (BD Biosciences, San Jose, CA) and analyzed with FlowJo software (TreeStar, Ashland, OR). Cell sorting was done on either Miltenyi autoMACS PROseperator or BD Influx (Rutgers Cancer Institute Flow Cytometry Shared Resource).

RNA-Seq analysis. Spleen cells from four ZEG20;FoxP3-RFP double reporter mice were sorted using the expression of GFP, RFP, CD62L, and CD4 to obtain highly pure (˜99%) populations of Zbtb20⁺ FoxP3⁺ Tregs, Zbtb20⁻ FoxP3⁺ CD62L^{Lo} Tregs, Zbtb20⁻ FoxP3⁺ CD62L^{Hi} Tregs. Approximately 1×10⁵ cells were resuspended in 100 L of TRIzol (Sigma-Aldrich) solution and stored at −80° C. until the RNA extraction. RNA was isolated from the cell using the Direct-zol RNA MicroPrep Kit (Zymo Research). RNA quality, SMARTer mRNA Amplification, libraries preparation, and RNA-Seq was done at the Lewis-Sigler Institute for Integrative Genomics, Princeton University. Sequencing was done on an Illumina HiSeq 2500 in Rapid mode as one lane of single-end 75 nt reads following the standard protocol. Raw sequencing reads were filtered by Illumina HiSeq Control Software and yielded about 160 million Pass-Filter (PF) reads for further analysis. PF Reads were de-multiplexed using the Barcode Splitter in FASTX-toolkit, and the reads from each library were mapped to the mouse genome. The htseq-count software was used next to obtain gene expression value as the total number of reads mapped to all exons of each gene, and these counts were further normalized to minimize the variation among samples and log 2-transformed after raising all zero values to 0.5 to obtain a log 2-fold change between each pair of samples. Heat maps were generated using GraphPad Prism (La Jolla, CA) software.

Quantitative RT-PCR. Cells were FACS sorted to obtain >98% pure populations of GFP⁺ and GFP⁻ cells. RNA purification and cDNA synthesis were carried out with the Qiagen RNeasy kit (gDNA shredder), and GoScript™ Reverse Transcription and random hexamers (Promega). The resultant cDNA was used to perform TaqMan (Life Technologies) based qPCR with TaqMan β2M (Mm00437762_m1) and IL-10 (Mm01288386) probes and TaqMan Universal PCR Master Mix No AmpErase UNG (Life Technologies). Samples were run on a QuantStudio6 Flex Real-Time PCR System (Life Technologies). Data were normalized to β2M and expressed as a relative target amount using the ΔΔCT method. zbtb20 expression in sorted GFP⁺ and GFP⁻ cells, collected from ZEG20 and zbtb20-cKO/GFP mice were assessed using Taq 2× Master Mix (NB BioLabs) and specific primers. The PCR products were run on 1% agarose (Hoefer) gel and imaged using Kodak's Gel Logic 200 Imaging System.

Western Blot. T cells and B cells from the spleen of ZEG20 mice were FACS sorted to obtain >98% pure populations of GFP⁺ and GFP⁻ cells. 150×10³ T cells and 25×10³ B cell were lysed with buffer containing 25 mM Tris HCl pH 7.6, 150 mM NaCl, 1% NP-40, 1% sodium deoxycholate, 0.2% SDS. An equal amount of protein lysates was separated by 8% SDS-PAGE gels and transferred to polyvinylidene fluoride membrane (Millipore). The membranes were probed with a primary monoclonal anti-Zbtb20 antibody (clone: 7C8) that L.M.C. generated by immunization of rats with overlapping peptides and Anti-b-Actin Clone AC-15 (Sigma A5441), as a loading control.

The T-cell line MT-2, acquired from the AIDS Research and Reference Reagent Program of the National Institutes of Health (Rockville, MD) and Jurkat T cell line (ATCC TIB-152), were grown in RPMI 1640 (Life Technologies) supplemented with 10% fetal bovine serum (Gemini Bio-Products), 100u/ml Penicillin/Streptomycin (Life Technologies), 2 mM GlutaMAX-1 (Life Technologies), and 10 mM HEPES (Life Technologies).

Nuclear lysates of MT-2 and Jurkat T cells were obtained by lysing cells in hypotonic lysis buffer (0.2% NP-40, 10 mM HEPES, 1.5 mM MgCl2, 10 mM KCl, 5 mM EDTA, and protease inhibitor cocktail); nuclei were then pelleted, resuspended with lysis buffer (20 mM HEPES, 300 mM NaCl, 20 mM KCl, 0.05% NP40, protease inhibitor cocktail) and an equal amount of lysates were separated by 8% SDS-PAGE gels and transferred to polyvinylidene fluoride membrane (Millipore). The membranes were probed with a primary monoclonal anti-Zbtb20 antibody (clone: 7C8) and anti-HDAC1 (Cell signaling), as a loading control.

Chromatin immunoprecipitation analysis. Chromatin immunoprecipitation (ChIP) was performed using ChromaFlash High-Sensitivity ChIP Kit (Epigentek p-2027) with anti-ZBTB20 (clone: 4A3) Rat IgG2a antibody (made by L.M.C.) and non-specific isotype control, anti-H2M 2C3M Rat IgG2a antibody. Briefly, cells were cross-linked with 1% formaldehyde (Sigma-Aldrich F8775), for 10 min at room temperature with gentle shaking, and subsequently quenched with 125 mM glycine. Next, cells were sonicated on ice and the fragmented chromatin was immunoprecipitated using 4 μg of antibodies. Real-time PCR was performed with PowerUp SYBR Green Master Mix (Thermo Fisher Scientific)). Data are presented as fold enrichment over control antibody. Three independent ChIPs were performed. Primers were chosen based on DNAse hypersensitivity and ChIP-seq data presented on the UCSC Genome Browser for the Human, February 2009 (GRCh37/hg/19) Assembly. Primer sequences are listed in Table 1.

TABLE 1
Primer sequences.

| SEQ ID | | | SEQ ID | |
|---|---|---|---|---|
| NO: | Name | Forward primer (5′-3′) | NO: | Reverse primer (5′-3′) |
| 1. | Exon 13 | CTAGAACGGAAGAAACCCAAG | | |
| | | ACA | | |
| 2. | Exon 14 | CTGACCTTGACCAGCAACACAC | 3. | AGCAAATGGAGCGCTACCTGTCC |
| | | AG | | A |
| | Exon 15 | | 4. | GGATTACCTTATCAAGCACATGGT |
| | | | | GAC |
| 5. | hIL10_-107 | GGAGGAGCTCTAAGCAGAA | 6. | AAGCCCCTGATGTGTAGAC |
| 7. | hIL10_-336 | CTAGGAACACGCGAATGAGAA | 8. | TTTAGGATGGGCTACCTCTCT |
| 9. | hIL10_-701 | GCAGACTACTCTTACCCACTTC | 10. | TGTGTTCCAGGCTCCTTTAC |
| 11. | hIL10_- | GAGGAAAGTAAGGGACCTCCT | 12. | CTTCTGTGGCTGGAGTCTAAAG |
| | 1032 | A | | |
| 13 | hIL10_- | AGTTTCTAGCAGGCTCTTTCTC | 14. | GGTGTCTCTTCCCAACTCTTC |
| | 1810 | | | |
| 15. | hIL10_- | CTTCAGCAAATGGCTTGAGATA | 16. | TGCTGAGATTACAGGCATGAG |
| | 2920 | AT | | |
| 17. | hIL10_- | GCTCTAATGGGAGGCAGATT | 18. | GTGTGTGTGTGTGTGCTTATG |
| | 4035 | | | |
| 19. | hIL10_- | GTGGAGACTGGAGCAAACTAA | 20. | GTCTGCATGACTACCTAGGATAA |
| | 6004 | | | C |
| 21. | hIL10_- | CGCCTGGAAGAGCACTAAT | 22. | TGAAGAAACCACTGTCCTTACA |
| | 8050 | | | |
| 23. | hIL10_- | GACAGGTCACTGGCAAATCA | 24. | CCAGGGTGGTCTTTCAGTTTAG |
| | 10218 | | | |
| 25 | hIL10_- | TCTTCTGATTTCCACTGCTCTC | 26. | TGTATTTCCTTCTCGCAATCCT |
| | 12113 | | | |

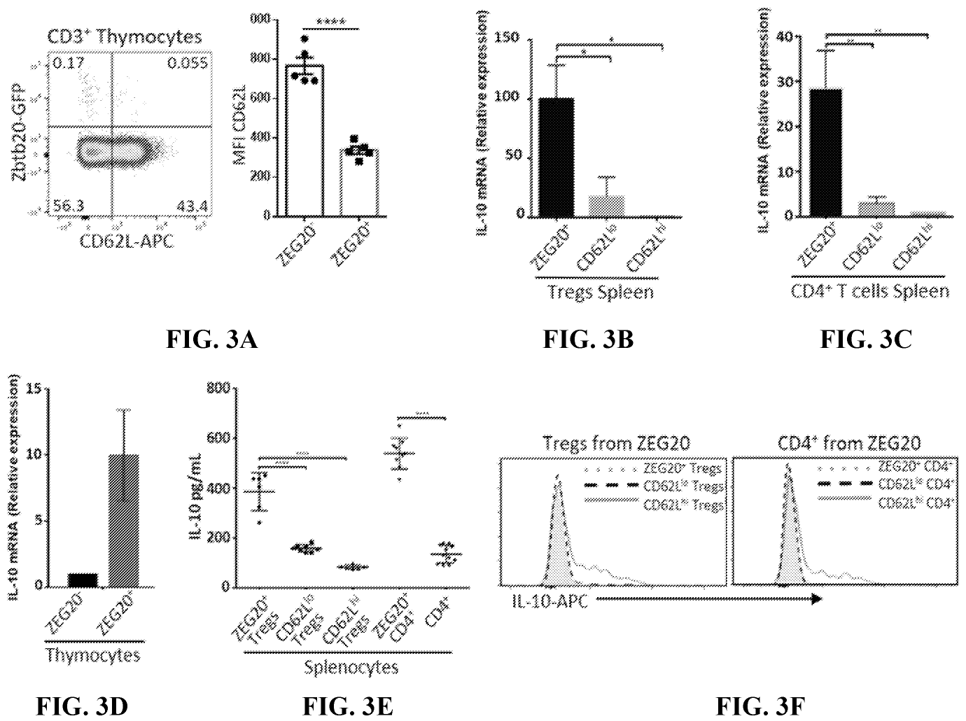

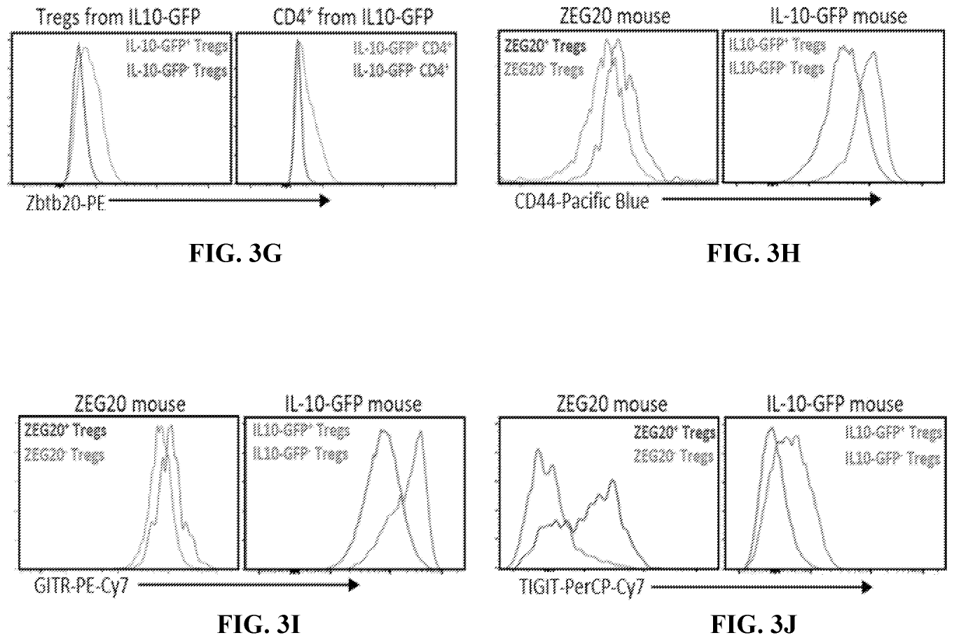

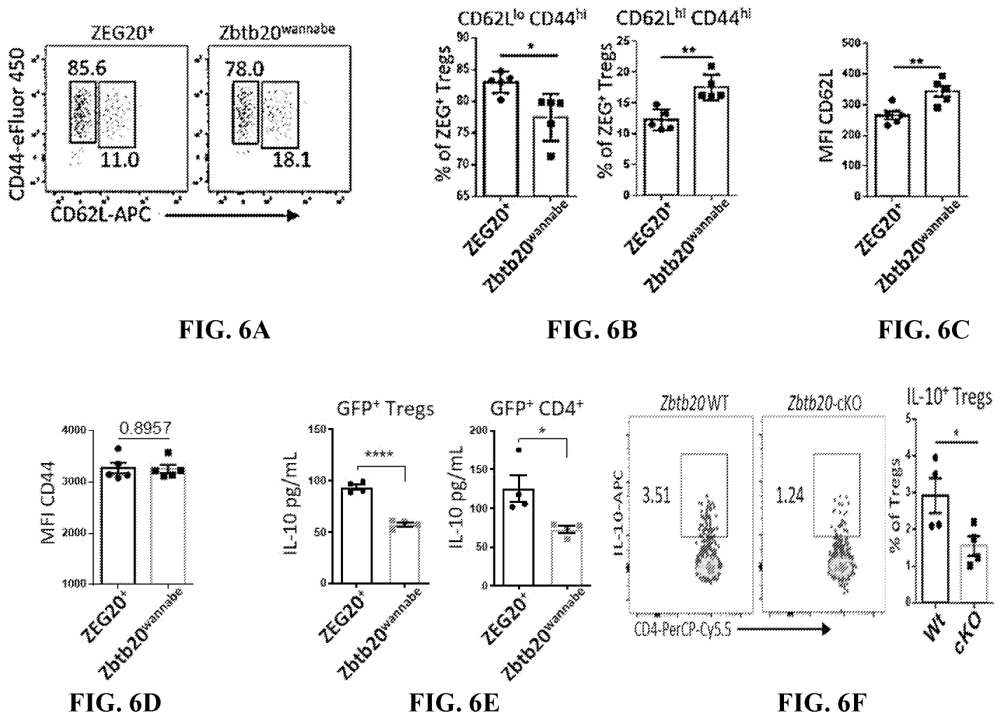

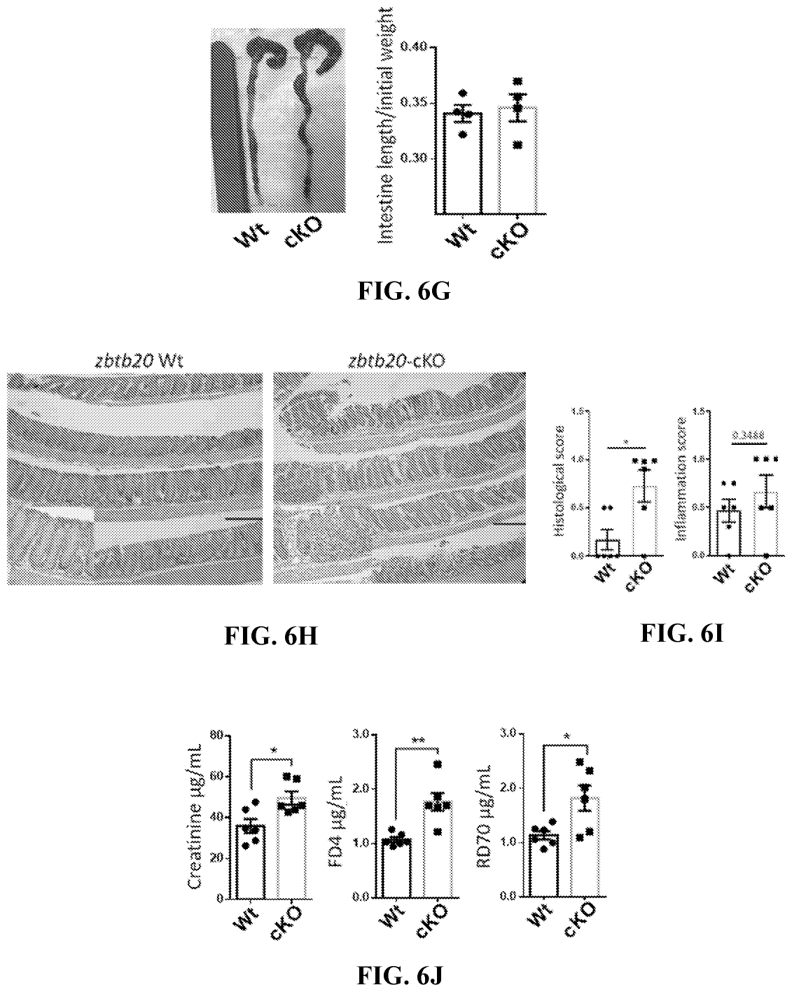

Cytokine Bead Array. Either 5×10⁴ (FIG. 6) or 2×10⁵ (FIG. 3) sorted T cells were stimulated in RPMI containing 5% HI FBS with Phorbol 12-myristate 13-acetate (PMA) (Sigma-Aldrich) (100 ng/mL) and ionomycin (Sigma-Aldrich) (500 ng/mL) for 24 hours and supernatants were collected. Blood samples for cytokine analysis were incubated for 1 hr at RT, spun down and serum was collected. The level of cytokines was determined with the BD Cytometric Bead Array (CBA) as per the kit's instructions. To measure the level of IL-6, IL-9, IL-17 in the serum, the WT and Zbtb20-cKO mice were injected intraperitoneally (i.p.) with 100 mg LPS (Sigma-Aldrich), the serum was collected after 21h post-injection as described above.

IL-10 Secretion Assay. IL-10 was measured using the Mouse IL-10 Secretion Assay (Miltenyi Biotec). 200×10⁵ cells were stimulated in RPMI containing 5% HI FBS with PMA (100 ng/mL) and ionomycin (500 ng/mL) at 37° C. for 3h. Next, the cells were briefly washed and incubated for an additional 45 min in the presence of an anti-IL-10 capture antibody that binds secreted IL-10 at the surface of the cell. Lastly, the cells were stained with APC-conjugated Mouse IL-10 detection antibodies, together with anti-CD25, anti-CD4, anti-CD3, and anti-CD62L for an additional 30 min. The IL-10 producing cells were detected by FACS.

Histology. The collection and preparation of the intestine for the histological analysis was done as described in Bialkowska et al., 2016. The colons of 8-week old zbtb20-cKO and WT mice were harvested and gently rinsed with modified Bouin's fixative (a mixture of 50% ethanol and 5% acetic acid in water) using a 10-mL syringe filled and a gavage needle. Swiss rolls were fixed in 10% buffered formalin overnight at room temperature, next rinsed and stored in 70% ethanol at 4° C. The embedding in paraffin, sectioning (5 m), and hematoxylin and eosin staining of the colons was done by Nationwide Histology LLC. The pathologists at Nationwide Histology LLC performed also blinded histological evaluation and scoring of the severity of the colitis as described in Koelink P J. et al., 2018, Kim J J. et al., 2012 and Erben U. et al. Disease severity was scored from 0 to 5, where 0 is a healthy colon and 5 severe colitis (histological and inflammation scores). The histological evaluation was further confirmed by a pathologist at the Rutgers Child Health Institute of NJ.

Intestinal permeability. The intestinal permeability assay was performed as described before by Edelblum K L et al., 2017. 14-week old zbtb20-cKO and WT mice were fasted for 2 hours and then gavaged with a mixture of 100 mg/mL creatinine (Sigma C4255), 80 mg/mL FITC-dextran 4 kDa (FD4) (Sigma 46944), and 20 mg/mL Rhodamine B-dextran 70 kDa (Sigma R9379). After 5h the 250-300 L of blood was collected by the retroorbital bleed. Samples were spun down at 1000 RPMs and ˜100 L of serum was collected for the analysis. Fluorescence intensity for FD4 and RD70 was determined by using a plate reader at 495 nm excitation/525 nm emission and 555 nm excitation/585 nm emission, respectively. The concentration of creatinine was measured using the Sciteck SVT creatinine kit (Sciteck 139-30) according to the manufacturer's protocol.

Dextran sodium sulfate treatment. The DSS-induced colitis model was done as described before. Mice received 3% colitis grade DSS (MP Biomedicals) in drinking water for 5 days and regular water for an additional 2-6 days. The body wight of the mice was assessed daily and presented as a percent of the initial weight. The presence of the occult blood in the stool was detected with the Hemoccult II test (Sensa).

Tregs adoptive transfer and colitis rescue experiments. Tregs from spleens were isolated using CD4⁺ CD25⁺ Regulatory T Cell Isolation Kit (Miltenyi Biotec) and autoMACS PROseperator. The cells were washed 3 times with sterile PBS and 5×10⁵ were resuspended in 200 μL of sterile saline. Tregs were next i.p. injected with a 1 mL sterile sub-Q syringe 26 G, one day before induction of the colitis with 3% DSS. Tregs subpopulations were sorted from the spleens of ZEG20;FoxP3-RFP double reporter mice. 1×10⁵ of the sorted cells were resuspended in 200 μL of sterile saline and i.p. injected into Zbtb20 cKO mice one day before induction of colitis.

Anti-CD40 IBD Mouse Model. At day 0, 100 μg of endotoxin-free anti-CD40 antibody (Clone FKG45, BioXCell) or anti-Rat IgG2a isotype control (Clone 2A3, BioXCell) were i.p. injected into RAG1^{-/-}, zbtb20-cKO, and WT mice. The progression of the disease was assessed by measuring body weight daily. Weight loss was presented as a percent of the initial weight.

In vitro induction of Zbtb20. 2×10⁶ of sorted GFP⁻ CD4⁺ T cells from ZEG20 mice were activated for 72h with plate-bound 5 μg/ml plate-bound anti-CD3 (2C11) and soluble anti-CD28. The cells were cultured in RPMI containing 5% HI FBS and 50 U/ml IL-2, supplemented with 20 ng/mL IL-6, 5 ng/mlLTGF-β, or both. After 72h cells were stained with anti-CD3, anti-CD4, anti-CD8, anti-CD62L, anti-MHC II and analyzed by FACS for the presence of GFP⁺ T cells.

In vivo induction of Zbtb20. GFP⁻ CD4⁺ CD45.2⁺ T cells from ZEG20;FoxP3-RFP double reporter mice were sorted and 3×10⁶ of the cells were i.p. injected into SJL mice (CD45.1⁺, non-GFP recipient). After 2 weeks, the recipient mice were sacrificed and lymphocytes from intestines were isolated and stained with anti-CD45.2, anti-CD45.1, anti-CD3, anti-CD4, anti-CD8, anti-CD62L, anti-MHC II and analyzed by FACS for the presence of GFP⁺ T cells.

Statistical analysis. Data from at least three samples in two or more independent experiments were collected as detailed in the FIGure legends. Statistical analysis was performed using GraphPad Prism (La Jolla, CA) software. All data were subjected to analysis with a two-tailed unpaired t-test or One-way ANOVA and are expressed as the mean; error bars represent ±SEM. P values <0.05 were considered significant. * P<0.05, ** P<0.01, *** P<0.001.

The Results the experiments disclosed herein are now described.

### Example 1: Zbtb20 is Expressed in Discrete T Cell Subsets

BTB-ZF genes are essential for the development or function of several types of leukocytes. Often, the expression of these genes is restricted to specific subpopulations. It was hypothesized, therefore, that discrete expression of BTB-ZF genes might be a means to identify new T cell effector populations. This screen was initiated by using microarrays to identify BTB-ZF transcription factor family members that changed their expression during the transition from double-positive to single positive during thymocyte development. This approach, showed, as expected, that ThPOK (zbtb7b) was expressed in CD4 single-positive (SP) thymocytes, but not in CD8 SP T cells and that PLZF (zbtb16) could be detected in the CD4⁺ SP and double negative (DN) subpopulations that contain NKT cells. Results also identified modest differences in zbtb20 expression during T cell maturation. In particular, zbtb20 was found to be more prominently expressed in CD4⁺ SP T cells. Zbtb20 previously has been shown to be expressed in B1 and germinal center B cells as well as in long-lived antibody-secreting cells in the bone marrow. Although not required for B cell development, Zbtb20 was shown to be necessary for plasma cell survival as mice with B cells deficient for zbtb20 were not able to generate long-term humoral immune responses. Zbtb20 has also been shown to function as a positive regulator of Toll-like receptor (TLR) signaling in myeloid cells. The expression and function of zbtb20 in T cells had not, however, been previously reported.

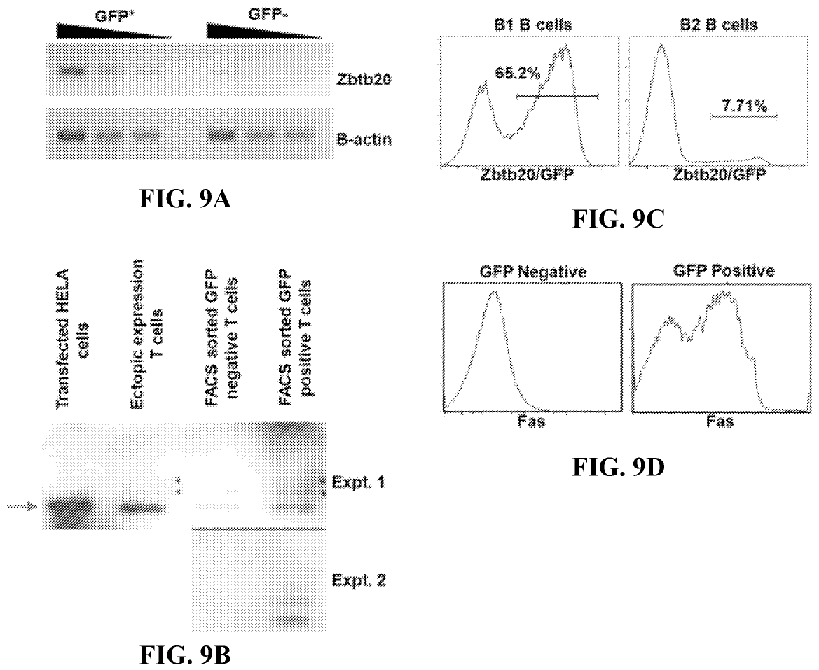

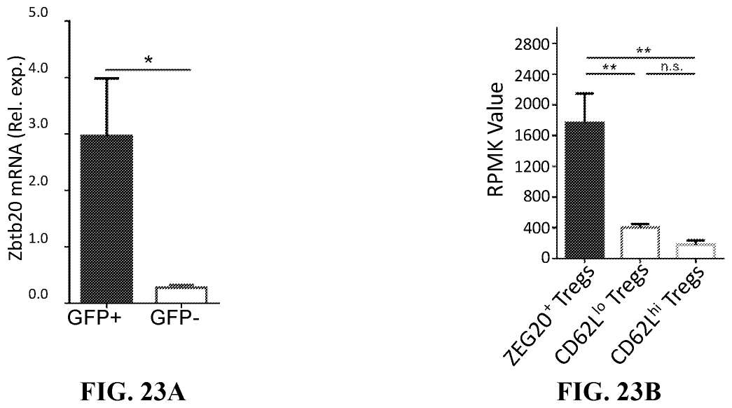

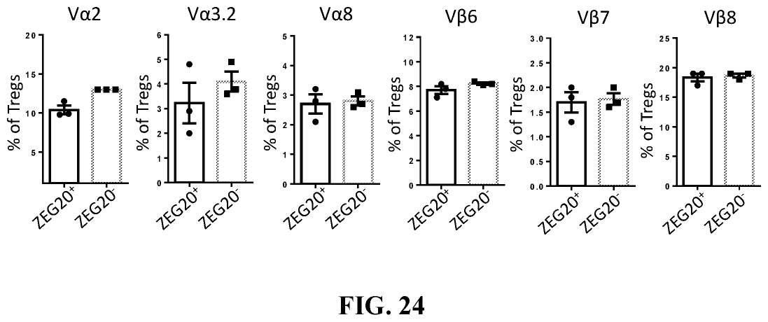

To study the single-cell expression of zbtb20, a ˜210 kb bacterial artificial chromosome (BAC) was obtained from the Gene Expression Nervous System Atlas (GENSAT). The bacterial artificial chromosomes (BAC) transgene was engineered with an enhanced green fluorescent protein (eGFP) gene immediately upstream of the ATG start codon of zbtb20. The inserted eGFP has its own start codon and polyadenylation signal and is under the control of the regulatory elements and the zbtb20 promoter region all encoded within the BAC. The design of the eGFP gene insertion prevented the expression of the BAC-encoded zbtb20 gene. GFP expression in these Zbtb20-eGFP (ZEG20) mice mimicked zbtb20 expression as shown by RT-PCR and, qPCR, RNA-Seq, and Western blot analysis of sorted GFP⁺ and GFP⁻ T cells (FIGS. 9A, 9B, 23A, 23B). Consistent with published reports of zbtb20 expression, GFP was found in most B1 B cells and some B2 B cells in the peritoneal cavity (FIG. 9C). In the spleen, only Fas⁺, germinal center B cells, were GFP⁺, which was also consistent with previously published data (FIG. 9D), confirming that expression of the reporter faithfully defined Zbtb20 expression.

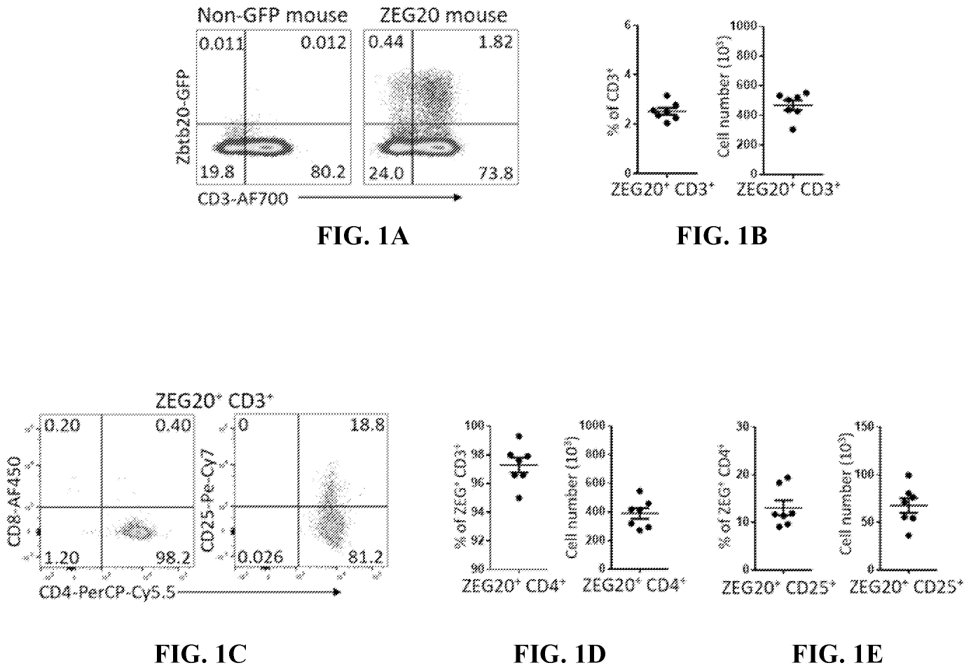

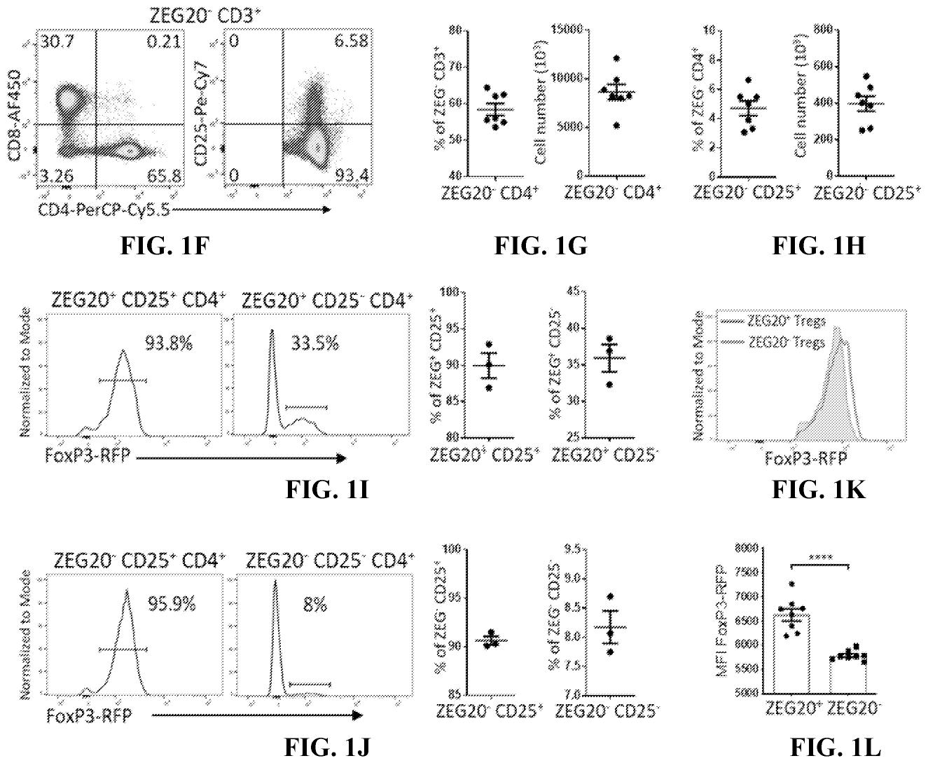

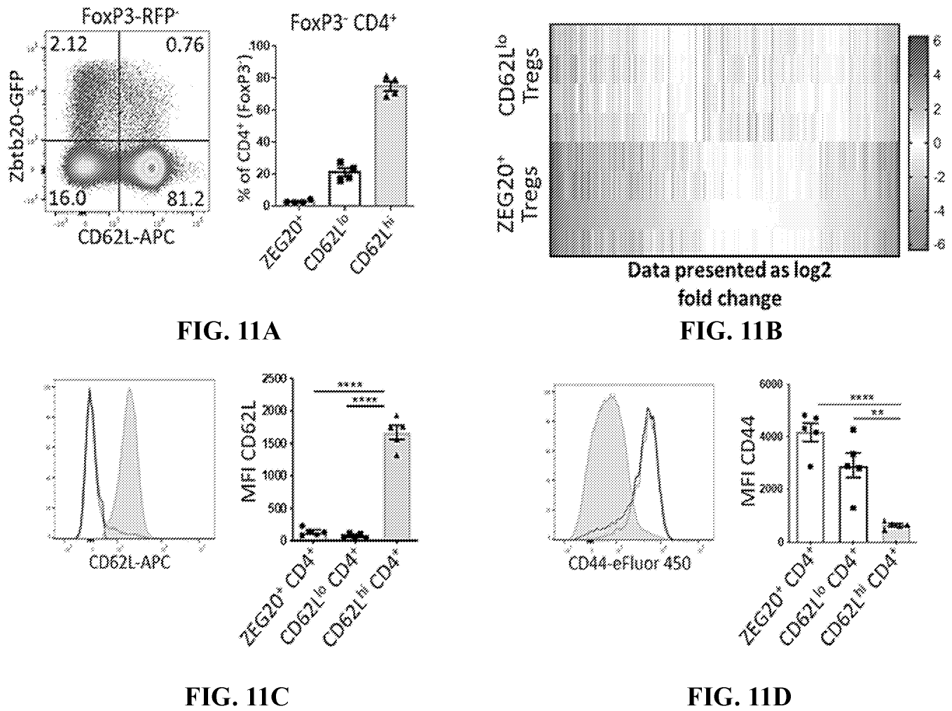

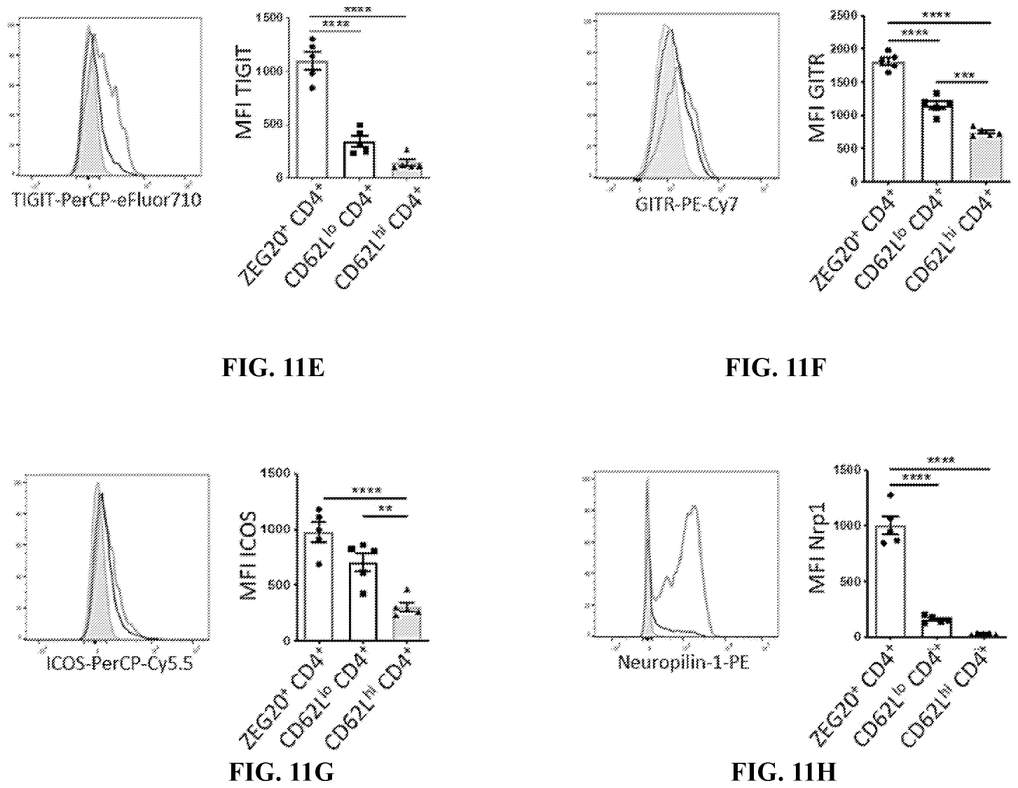

Zbtb20 expression, as detected by GFP expression in cells from ZEG20 mice, was found in ˜3% of CD3⁺ spleen T cells (ZEG20⁺ T cells) (FIGS. 1A, 1B). These cells were all CD4⁺ T cells and ˜20% of them expressed CD25 (FIGS. 1C-1H). To assess if the CD25⁺ ZEG20⁺ T cells also expressed the Treg lineage-specific transcription factor, FoxP3, ZEG20 mice were crossed with FoxP3-RFP reporter mice. Analysis of spleen cells from these double reporter mice showed that nearly all of both the ZEG20⁺ and ZEG20⁻ CD25+CD4⁺ T cells expressed FoxP3 (FIGS. 1I, 1J). Interestingly, ZEG20⁺ Tregs had higher expression of FoxP3-RFP as compared to ZEG20⁻ Tregs (FIGS. 1K, 1L) suggesting these are functionally different subsets of cells. Additionally, more than 30% of ZEG20⁺ CD25⁻ CD4⁺ T cells also expressed FoxP3, whereas only 8% of ZEG20⁻ CD25⁻ CD4⁺ T cells expressed the transcription factor (FIGS. 11, 1J). Similar results were obtained by direct staining of FoxP3 in sorted ZEG20⁺ CD25⁺ and ZEG20⁺ CD25⁻ T cells (FIG. S2A, B). In total, approximately 50% of ZEG20⁺ T cells express FoxP3. Thus, analysis of single-cell expression of Zbtb20 enabled the identification of two T cell populations: Zbtb20⁺ Tregs and Zbtb20⁺ CD4⁺ T cells.

### Example 2: Zbtb20 Expression Defines Phenotypically Distinct Subsets of CD4⁺ T Cells of which Half Also Express FoxP3

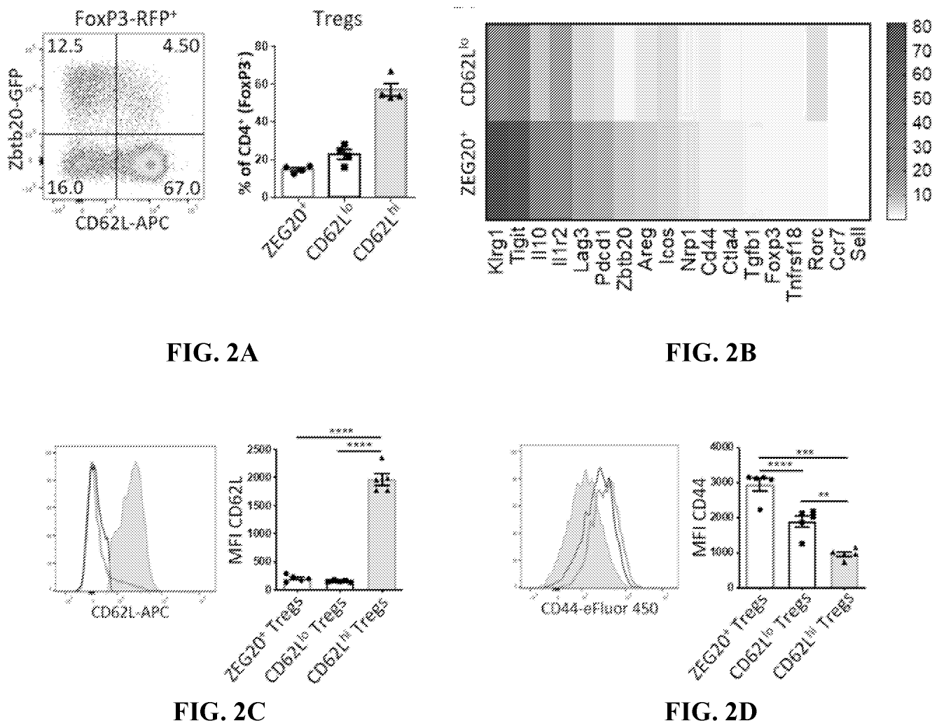

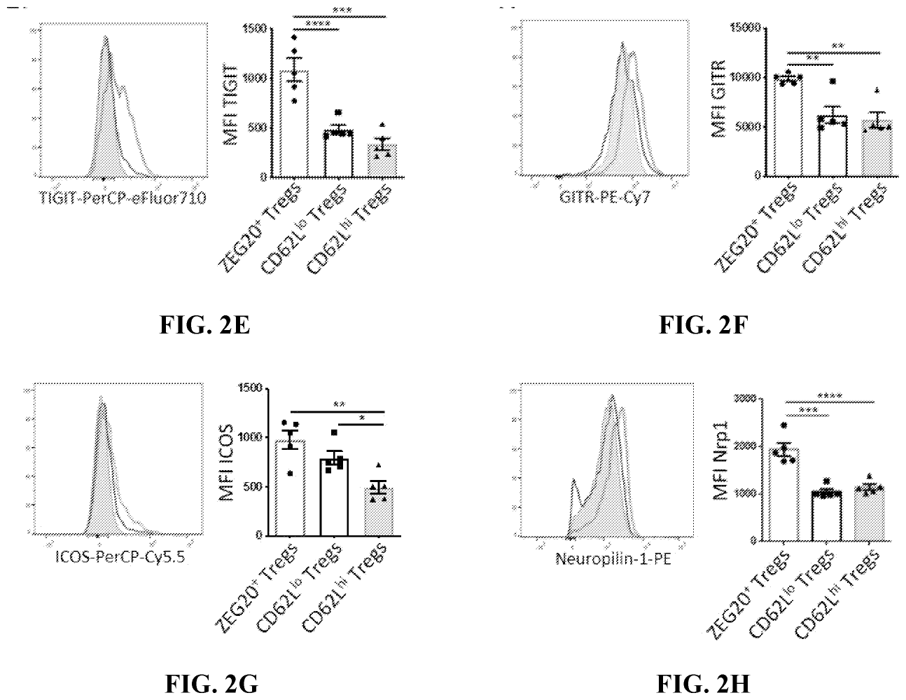

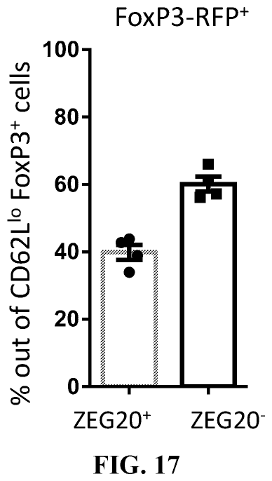

CD62L is an adhesion molecule that is typically shed by naïve T cells following activation. In the spleen, about 30% of CD25⁺ CD4⁺ T cells express low levels of CD62L. Studies suggest that differences in the expression of CD62L correlate with differences in the function of Tregs. The present studies found that more than 75% of ZEG20⁺ Tregs were CD62L^{lo} (FIGS. 2A, 17). Loss of CD62L expression was not, however, a marker for ZEG20⁺ Tregs, since fewer than half of the total CD62L^{lo} Tregs expressed the transcription factor. Approximately 75% of FoxP3⁻ ZEG20⁺ T cells were also found to be CD62L^{lo} (FIG. 11A, 17).

To further study these cells, RNA-seq analysis was performed on purified ZEG20⁺ Tregs, ZEG20⁻ CD62L^{Lo} Tregs, and ZEG20⁻ CD62L^{hi} Tregs sorted from ZEG20;FoxP3-RFP double reporter mice. Only samples with high purity (˜99%) were used for RNA isolation, library generation, and sequencing. These studies focused on the comparison of the two populations of cells that similarly expressed low levels of CD62L. The RNA-seq analysis showed that ZEG20⁺ Tregs have a gene expression profile that is distinct from the ZEG20-CD62L^{lo} Tregs with 109 genes expressed at higher levels and 85 genes expressed at lower levels (FIG. 11B). Several genes, known to be associated with Treg function, including TIGIT, Tnfrsf18 (GITR), Klrg1, ICOS, Pdcd1 (PD-1), Lag3, Nrp1 were differentially expressed (FIG. 2B). Interestingly, these studies also found that Il10 was expressed in the ZEG20⁺ Tregs.

FACS analysis further confirmed that the ZEG20⁺ Tregs had a distinct phenotype. For example, ZEG20⁺ Tregs had the highest expression of CD44, TIGIT, GITR, ICOS as compared to CD62L^{lo} or CD62L^{hi} Tregs (FIGS. 2D-2G). Similarly, ZEG20⁺ CD4⁺ T cells also had the highest expression of these markers when compared to CD62L^{lo} and CD62L^{hi} CD4⁺ T cells (FIGS. 11D-11G). Hence, the RNA-seq and FACS analysis both showed that Zbtb20 expressing T cells are phenotypically and transcriptionally distinct from non-Zbtb20 expressing T cells.

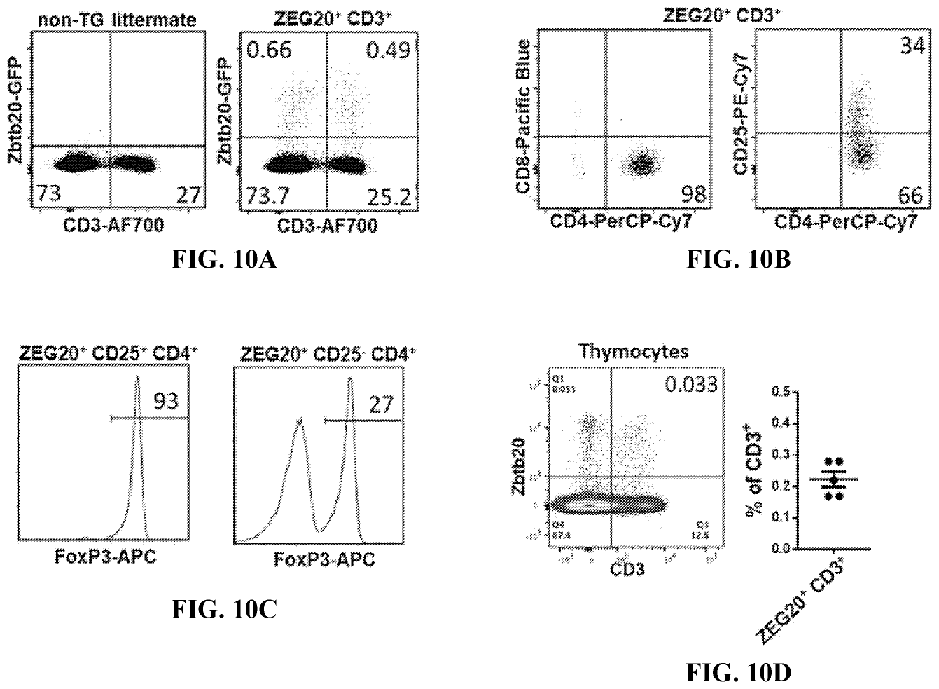

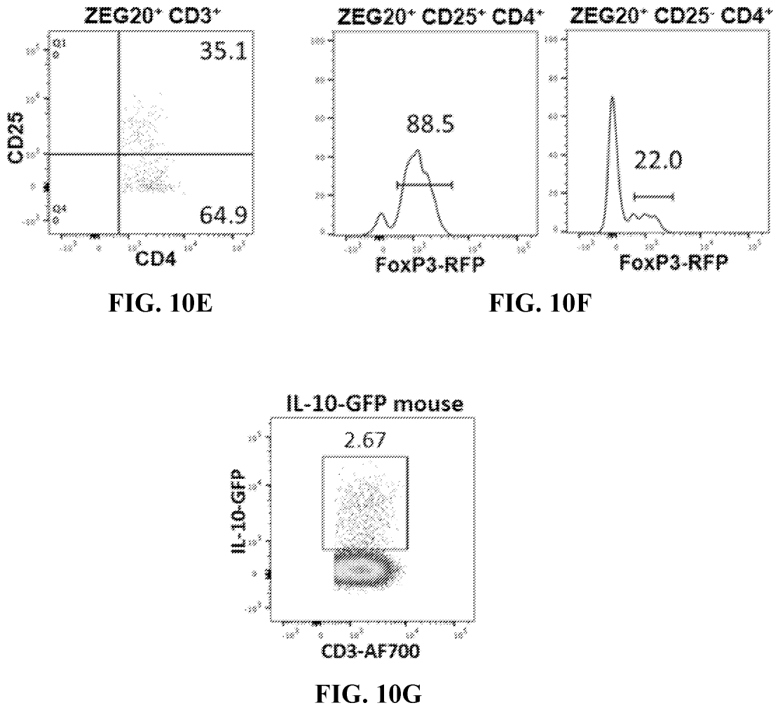

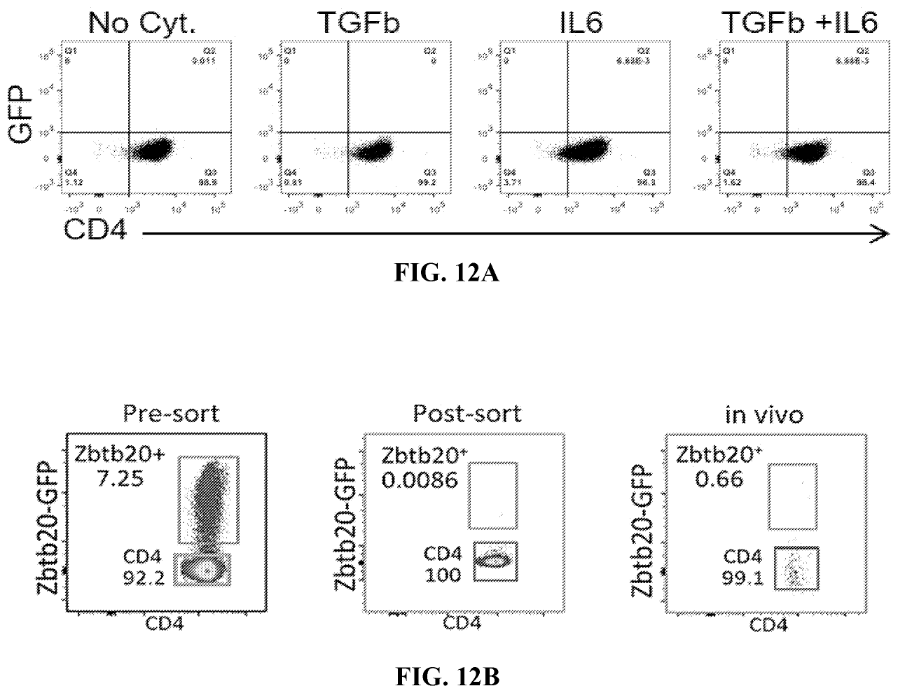

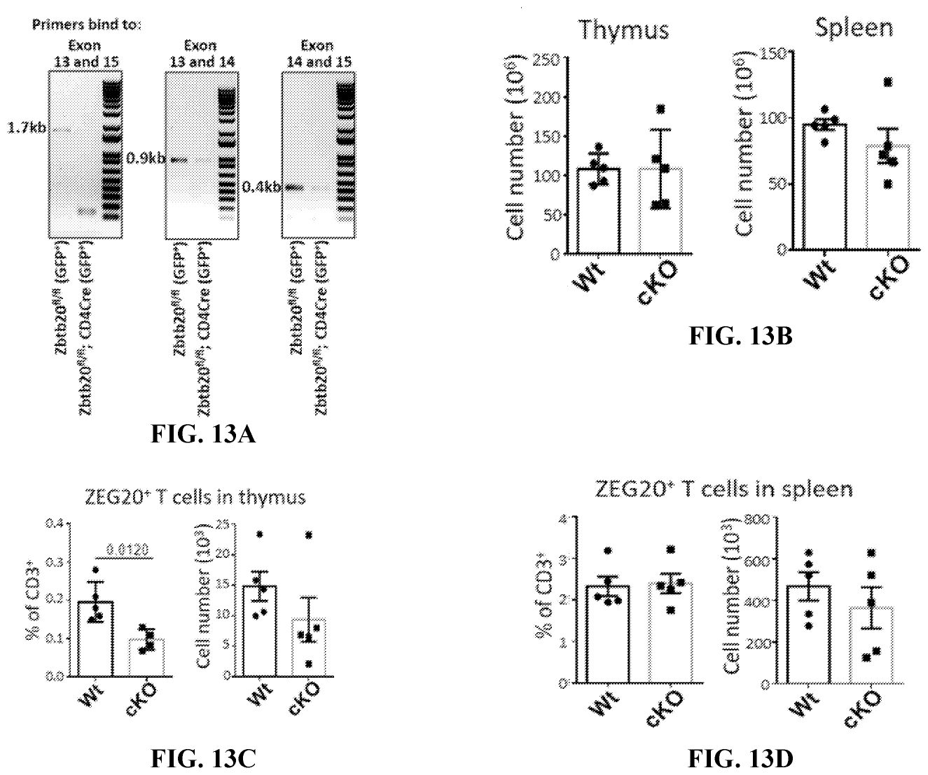

Neuropilin 1 (NRP1), which is considered to be a marker of thymus-derived Tregs, was expressed at higher levels in ZEG20⁺ Tregs as compared to the ZEG20⁻ populations, as shown both by RNA expression levels and by FACS (FIG. 2H). ZEG20⁺ CD25⁻ CD4⁺ T cells also expressed NPR1 whereas both CD62L^{lo} and CD62L^{hi} CD4⁺ T cells were negative (FIG. 11H). Expression of NRP1 on Zbtb20 expressing cells suggested that these cells developed in the thymus. Consistent with this observation, ZEG20⁺ FoxP3⁺ CD3^{hi} CD4 single-positive thymocytes were detected in double reporter mice. Similar to the spleen, approximately 30% of the thymic Tregs expressed Zbtb20-GFP (FIGS. 10D-10F). Furthermore, the ZEG20⁺ Tregs in the thymus also expressed low levels of CD62L (FIG. 3A). These data show that both Zbtb20 expression and the activated phenotype are induced during development, suggesting that Zbtb20 expressing Tregs are a distinct subset rather than a derivative of conventional Tregs that acquire these distinct features in the periphery. Consistent with this, Zbtb20 was not induced in sorted ZEG20⁻ CD4⁺ T cells by activation with anti-CD3 and anti-CD28 in vitro even when exogenous TGFβ and IL-6 were added (FIG. 12A). Also, Zbtb20 was not expressed in ZEG20⁻ T cells 2 weeks following intraperitoneal injection into mice (FIG. 12B). Thus, Zbtb20⁺ Tregs and Zbtb20⁺ CD4⁺ T cells develop in the thymus and are, potentially, distinct lineages of T cells.

### Example 3: Zbtb20 Expressing T Cells Constitutively Express Il10

Tregs use several regulatory mechanisms to control the host immune response, but of particular importance in the intestine is the secretion of IL-10. RNA-Seq analysis of mRNA collected from double reporter mice showed that directly ex vivo, ZEG20⁺ Tregs had substantial levels of Il10 mRNA. “Pre-formed” mRNA such as this is a hallmark feature of NKT cells and is one of the reasons for their innate-like ability to rapidly produce cytokines almost immediately after activation. Il10 message in Zbtb20 expressing T cells suggested that analogous to NKT cells, these cells might also rapidly produce cytokine. To test this, ZEG20⁺ and ZEG20⁻ Tregs were sorted from the spleens of ZEG20 mice. The ZEG20⁻ cells were further divided into CD62L^{lo} and CD62L^{hi} Treg populations. ZEG20⁺ CD25⁻ CD4⁺ T cells and CD62L^{lo} and CD62L^{hi} CD25⁻ ZEG20⁻ CD4⁺ T cells were also collected. The mRNA from the sorted cells was immediately isolated, reverse transcribed and the resultant cDNA was used to perform TaqMan based qPCR. ZEG20⁺ Tregs and ZEG20⁺ CD4⁺ T cells had a 100-fold and 30-fold higher Il10 expression, respectively, as compared to their ZEG20-counterparts (FIGS. 3B-3C). qPCR of cDNA generated from sorted thymocytes, also showed that Il10 mRNA was highly expressed in the thymic ZEG20⁺ cells in comparison to ZEG20-cells (FIG. 3D). The presence of preformed Il10 mRNA in the antigen inexperienced ZEG20⁺ thymocytes indicates that the constitutive expression of the cytokine is an intrinsic characteristic of these cells, acquired during their development in the thymus.

Next, the ability of FACS sorted ZEG20⁺ Tregs and ZEG20⁺ CD4⁺ T cells to secrete IL-10 was compared after 24h stimulation with PMA and ionomycin. The level of IL-10 secreted by ZEG20⁺ Tregs, ZEG20⁻ CD62L^{lo} Tregs, ZEG20⁻ CD62L^{hi} Tregs, ZEG20⁺ CD4⁺ T cells, and ZEG20⁻ CD4⁺ T cells was measured by using BD Cytometric Bead Array (CBA). Consistent with the presence of Il10 mRNA, both ZEG20⁺ Tregs and ZEG20⁺ CD4⁺ T cells produced 3- and 5-fold higher amounts of IL-10, respectively, after primary stimulation as compared to ZEG20⁻ Tregs and ZEG20⁻ CD4⁺ T cells (FIG. 3E).

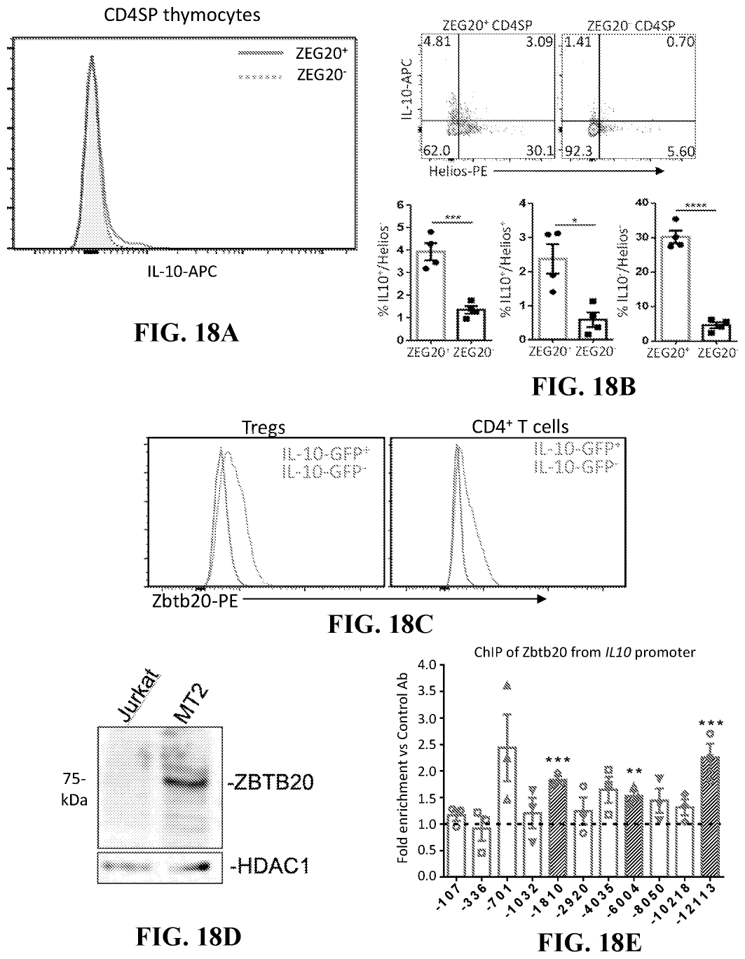

The ability of ZEG20⁺ T cells to produce IL-10 was further demonstrated using an IL-10 cytokine secretion capture assay (Miltenyi Biotec). Splenocytes from ZEG20 mice were activated with PMA and ionomycin in complete medium at 37° C. for 3h then briefly washed and incubated for an additional 45 min in the presence of an anti-IL-10 antibody that “captures” IL-10 at the surface of secreting cells. Data showed that ZEG20⁺ T cells were capable of rapid IL-10 secretion after just 3h stimulation (FIG. 3F, 18). This near-immediate production of IL-10 was also evident for ZEG20⁺ thymocytes (FIG. 18A).

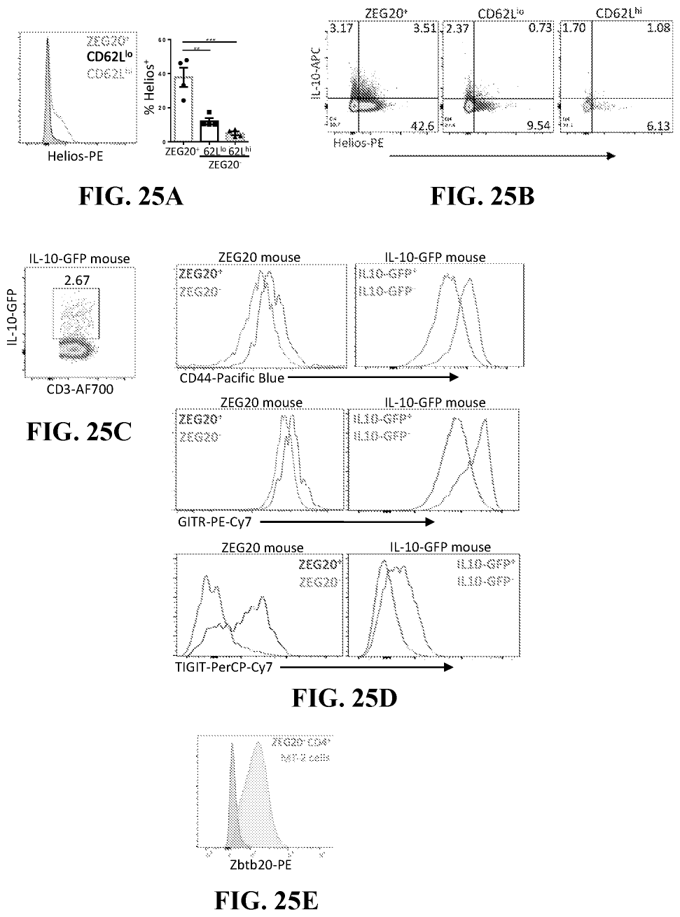

The transcription factor Helios has been proposed as a marker of thymic derived Tregs that produce high levels of IL-10 and are highly immunosuppressive. More than 30% of ZEG20⁺ T cells in the thymus (FIG. 18B) and the spleen (FIGS. 25A, 25B) expressed Helios. Both Helios⁺ and Helios⁻ ZEG20⁺ T cells, however, were able to rapidly secrete IL-10 (FIGS. 18B and 25B).

FACS analysis of IL-10-GFP reporter mice revealed a small percentage (˜3%) of CD3⁺ T cells in the spleen expressed GFP⁺. IL-10-GFP⁺ Tregs, IL-10-GFP⁻ Tregs, IL-10-GFP⁺ CD4⁺ T cells, and IL-10-GFP⁻ CD4⁺ T cells were sorted and evaluated for whether constitutive Il10 expression correlated with zbtb20 expression. Direct staining of the Zbtb20 protein clearly showed that IL-10-GFP⁺ cells expressed the transcription factor (FIG. 3G, 19A). Furthermore, the IL-10-GFP⁺ T cells had a phenotype similar to ZEG20⁺ Tregs, with higher expression of CD44, GITR, and TIGIT as compared to non-GFP Tregs (FIGS. 3H-3J). These data provided additional evidence that zbtb20 expression correlates with an open Il10 locus that is transcriptionally competent.

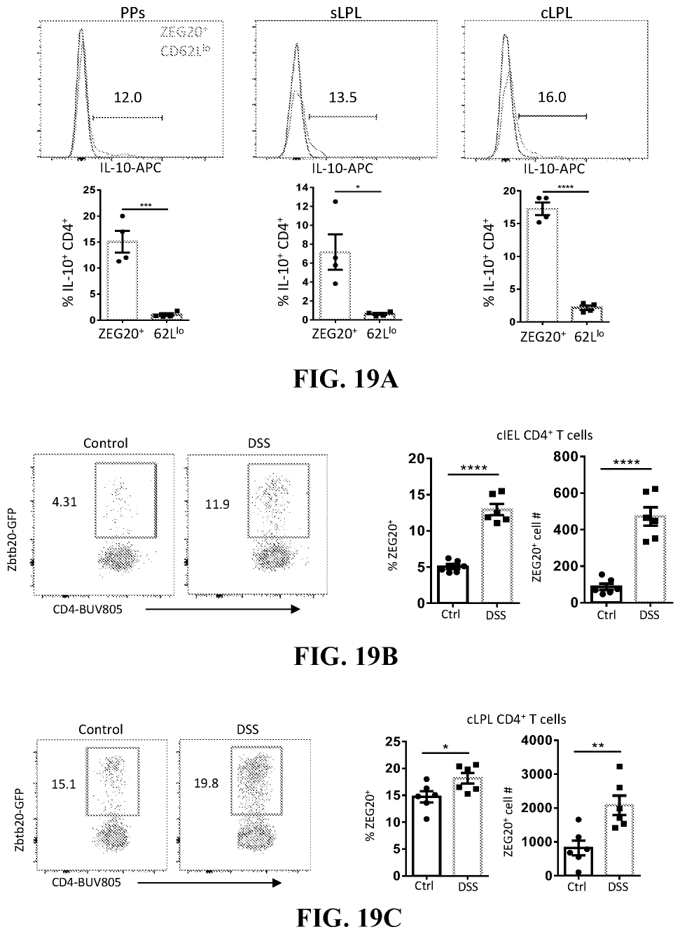

FACS analysis of IL-10-GFP reporter mice revealed that a small percentage (˜3%) of CD3⁺ T cells in the spleen expressed GFP (FIG. 25C). We sorted IL-10-GFP⁺ Tregs, IL-10-GFP⁻ Tregs, IL-10-GFP⁺ CD4⁺ T cells, and IL-10-GFP⁻ CD4⁺ T cells and stained them for Zbtb20 protein expression. Both the Tregs and CD4⁺ T cells that were IL-10-GFP⁺ expressed the Zbtb20 (FIG. 19A). Furthermore, the IL-10-GFP⁺ Tregs had a phenotype similar to ZEG20⁺ Tregs, with higher expression of CD44, GITR, and TIGIT as compared to non-GFP Tregs (FIG. 25D).

Finally, chromatin immunoprecipitation (ChIP) assays were used to determine if Zbtb20 binds the IL10 promoter. A human Treg-like T cell line was identified, MT-2, that expresses FoxP3 and IL-10. FACS and Western blot analysis showed that MT-2 also expressed Zbtb20 (FIG. 25E, 18D). Proteins crosslinked to the chromatin were precipitated with a monoclonal antibody specific for Zbtb20. qPCR was performed using primers amplifying 11 regions 5′ of the IL-10 mRNA transcription start sites, previously identified as accessible to transcription factor binding. Three regions, located at positions −1,810, −6,004, and −12,113 were enriched as compared to an IP with an irrelevant antibody (FIG. 18E). Altogether, these data show that Zbtb20 expressing Tregs and Zbtb20 expressing CD4⁺ T cells constitutively make Il10 mRNA and have the ability to rapidly secrete IL-10 following the primary stimulation.

### Example 4: Zbtb20 Expressing Tregs are Enriched in the Gastrointestinal Tract

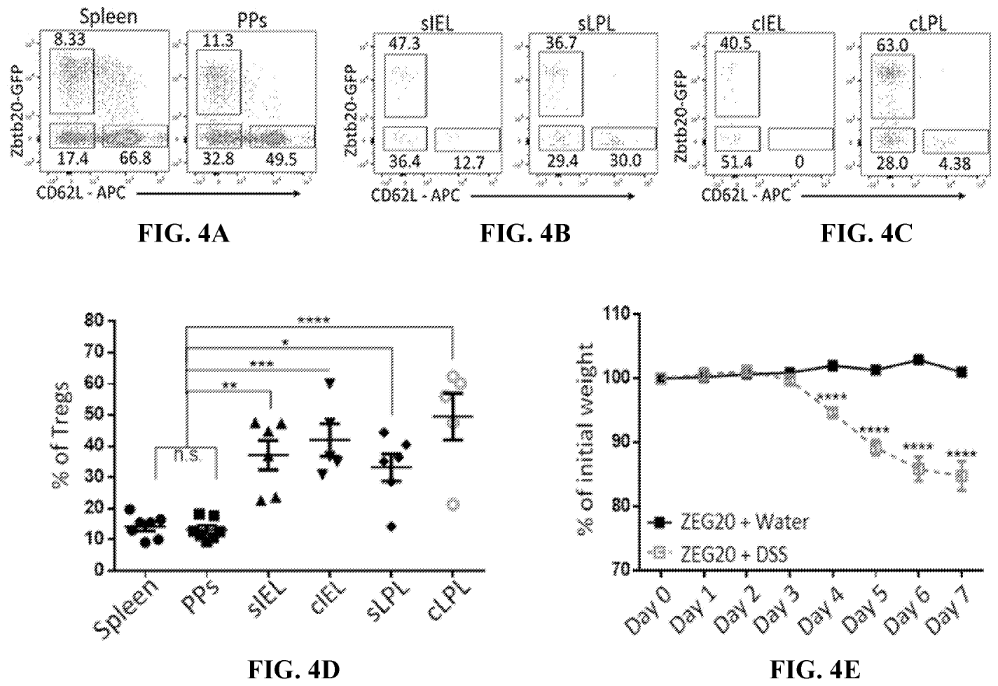

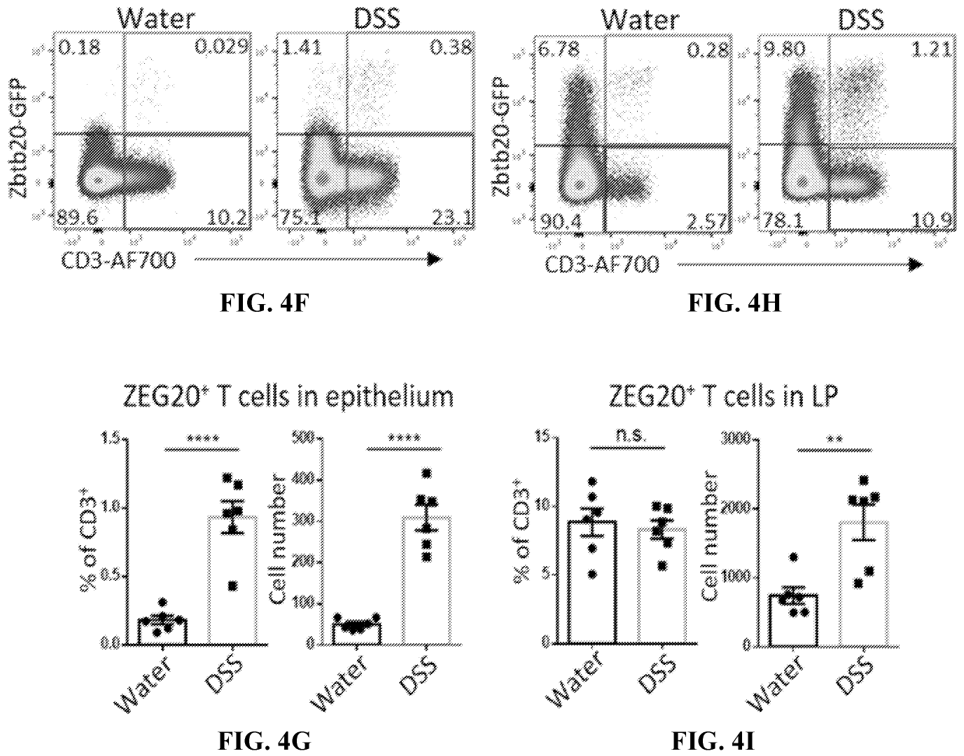

IL-10 plays a pivotal role in the maintenance of intestinal homeostasis. Therefore, the frequency of ZEG20⁺ T cells in the Peyer's Patches (PPs), the epithelium of the small intestine (intraepithelial lymphocytes—sIEL), and colon (cIEL) and the lamina propria of the small intestine (sLPL) and colon (cLPL) was determined (FIG. 4A-C). FACS analysis showed that up to 40% of Tregs in the epithelium (sIEL and cIEL) in the small intestine and colon were ZEG20⁺, as compared to ˜15% ZEG20⁺ Tregs in the spleen from the same mice. The greatest accumulation of ZEG20⁺ Tregs were found in LP particularly in the colon (cLPL) where more than 50% were Zbtb20⁺. The frequency of ZEG20⁺ Tregs in the Peyer's Patches, however, was similar to the spleen (FIG. 4D). Importantly, similarly, to ZEG20⁺ T cells in the spleen and thymus, intestinal ZEG20⁺ T cells also rapidly secrete IL-10 (FIG. 19A).

Acute colitis was induced in ZEG20 mice by the use of 3% dextran sodium sulfate (DSS) in the drinking water for 5 days followed by a 3 day recovery period. The change in body weight due to the progression of the colitis was determined daily (FIG. 4E). On day 8, the DSS treated and control mice were sacrificed and lymphocytes from the colon epithelium and LP were isolated and assessed by FACS. The induction of colitis led to a 6-fold increase in frequency and numbers of intraepithelial ZEG20⁺ T cells and a 3-fold increase in both the frequency and absolute numbers of intraepithelial ZEG20⁺ CD4⁺ T cells (FIG. 19B) and a ˜20% increase in the percentage and a 2.5-fold increase in the absolute number of ZEG20⁺ CD4⁺ T cells in LP of the DSS treated mice (FIGS. 4F-4G, 19C). Although the percentage of ZEG20⁺ T cells did not change in LP of the DSS treated mice, there was a 2.5-fold increase in the absolute number of ZEG20⁺ T cells (FIGS. 4H-4I). Therefore, intestinal ZEG20⁺ T cells increased in number during acute colitis, suggesting that they are an active participant in the regulation of the disease.

### Example 5: Phenotype and Function are Altered in Zbtb20 Conditional Knock Out Mice

Mice were generated with a zbtb20 allele flanked by loxP sites using conventional methods of homologous recombination in embryonic stem (ES) cells derived from C57BL/6 mice. T cell-specific deletion of zbtb20 was induced during thymocyte development by the use of a CD4-Cre transgene. Next, the Zbtb20-eGFP reporter (ZEG20) was bred into these conditional knockout mice with the expectation that eGFP expression would mark cells that should express Zbtb20, but cannot since the gene was deleted. Importantly, the ZEG20 BAC transgene was constructed such that Zbtb20 is not translated. A similar strategy has been used to study FoxP3 deficient Tregs. Thus, analysis of these “Zbtb20^{wannabe}” T cells collected from ZEG20;CD4-Cre;zbtb20fl/fl (zbtb20-cKO/GFP) mice provided an ideal means to study the impact of the loss of zbtb20 expression specifically in the T cells that should express the transcription factor.

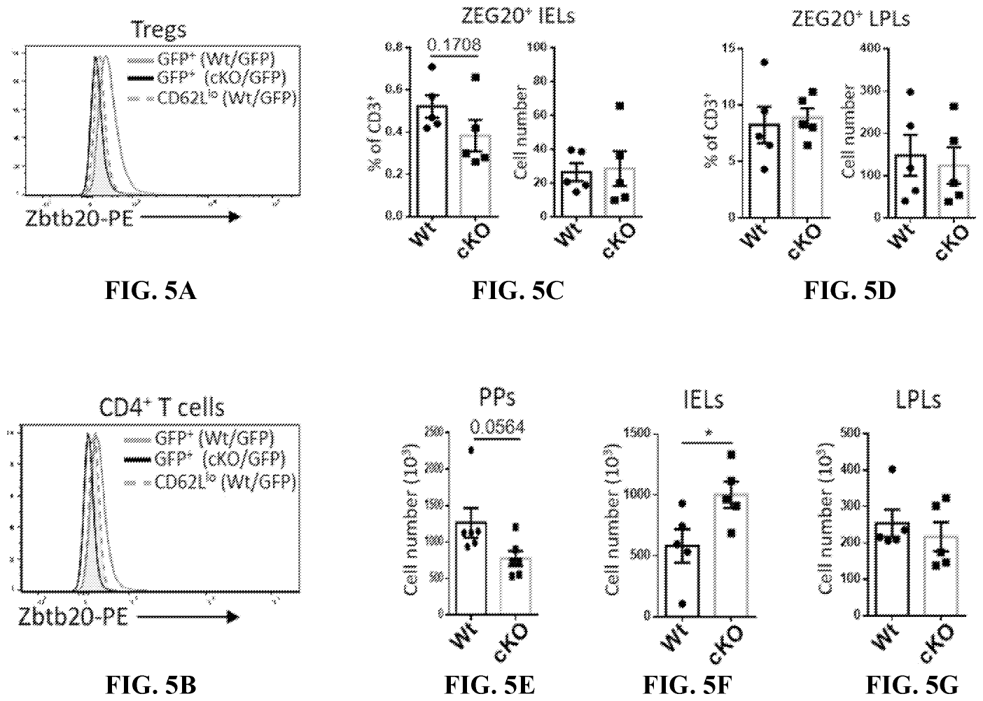

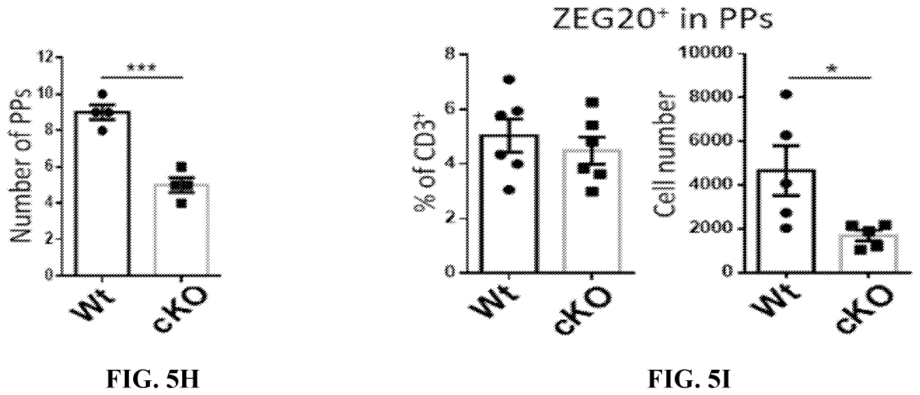

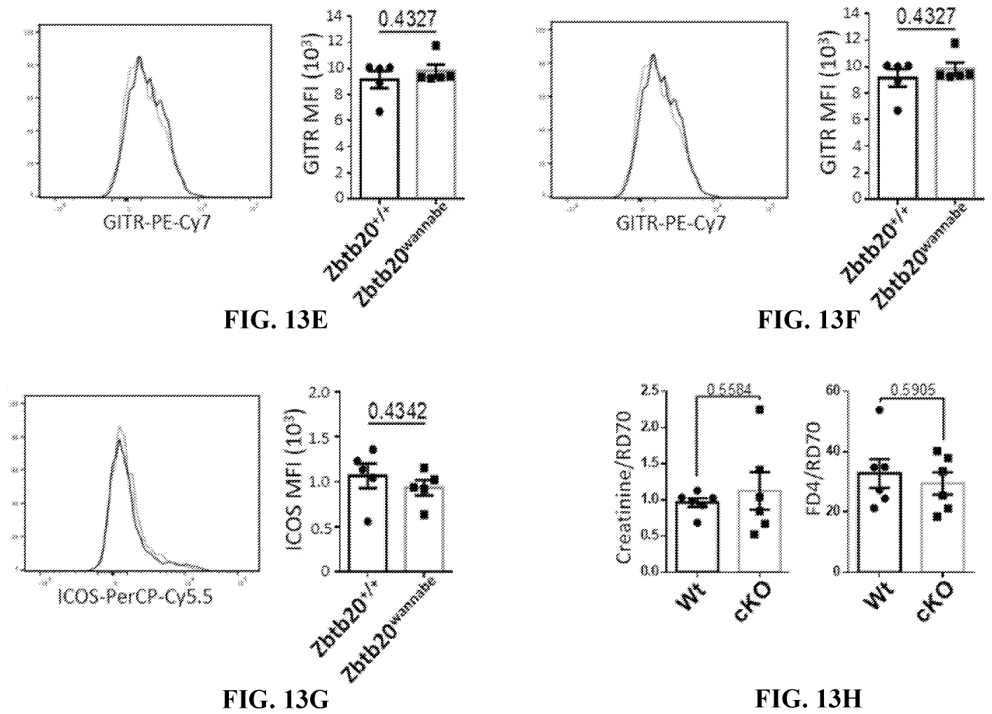

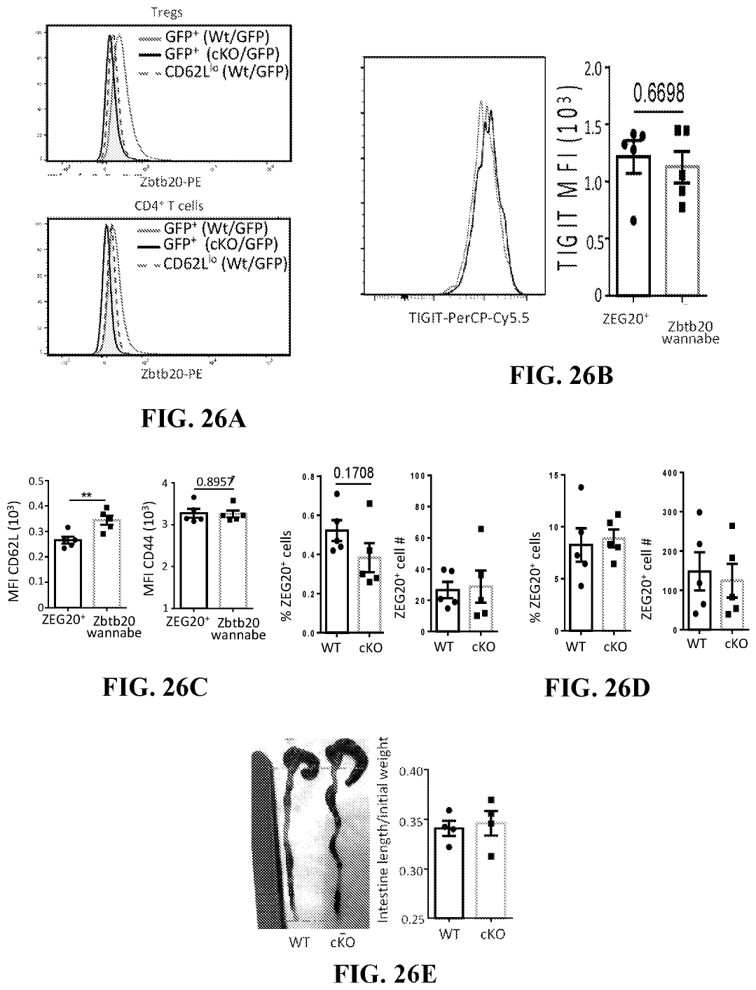

To confirm that the zbtb20 gene was deleted and that protein was not made, the GFP⁺ and GFP⁻ Tregs and CD4⁺ T cells were collected from zbtb20-cKO/GFP (cKO/GFP) and zbtb20^{fl/fl}/GFP (WT/GFP) littermates. RNA from the sorted cells was isolated and reverse transcribed. PCR showed that the exon flanked by loxP sites was deleted in zbtb20-cKO/GFP mice (FIG. 13A). Next, FACS was done on the GFP⁺ and GFP⁻ T cell subsets that had been sorted from zbtb20-cKO/GFP and zbtb20^{fl/fl}/GFP mice. Staining with the anti-Zbtb20-PE antibody showed that Zbtb20^{wannabe} cells did not express the Zbtb20 protein (FIGS. 5A, 5B, 26A). Combined, these data show that conditional deletion of the gene was successful.

The thymuses, spleens, and intestines from 8-week-old zbtb20-cKO/GFP mice were analyzed to assess changes resulting from the loss of Zbtb20 expression. Overall, there was no difference in the total lymphocyte numbers in thymuses and spleens of the cKO mice when compared to sex-matched littermates that did not express CD4-Cre (Zbtb20 WT mice) (FIG. 13B). The frequency of Zbtb20^{wannabe} T cells in the thymus of zbtb20-cKO/GFP mice was, however, decreased by half (FIG. 13C). There also was a small reduction of Zbtb20^{wannabe} T cells in the epithelium of the colon but no difference in the frequency of these cells in LP (FIGS. 5C, 5D). Further analysis of colons from zbtb20-cKO/GFP mice showed an approximately 40% decrease in the number of leukocytes in PPs and an 80% increase of total leukocyte numbers in the epithelium as compared to WT animals (FIGS. 5E, 5F). Interestingly, zbtb20-cKO/GFP mice had approximately half as many PPs as WT and the number of Zbtb20^{wannabe} T cells in the PPs was also 60% reduced (FIGS. 5H, 5I).

In the spleens of zbtb20-cKO/GFP mice a 10% decrease in the frequency of CD62L^{lo} Zbtb20^{wannabe} Tregs was observed but no difference in surface expression of CD44, GITR, TIGIT, or ICOS (FIGS. 6A-6D; 13E-13G, 26B, 26C). Next, sorted Zbtb20^{wanabes} Tregs, Zbtb20^{wannabes} CD4⁺ T cells, ZEG20⁺ Tregs, and ZEG20⁺ CD4 T cells were activated with PMA and ionomycin. The medium was collected after 24h and IL-10 levels were measured by CBA. Consistent with a potential role for Zbtb20 in regulating the production of IL-10, the Zbtb20^{wannabe} Tregs and Zbtb20^{wannabe} T cells collected from the zbtb20-cKO/GFP mice secreted ˜40% less IL-10 as compared to the WT controls (FIG. 6E). Lastly, Tregs collected from zbtb20-cKO and WT mice were activated and assessed for their ability to rapidly produce IL-10; 3h post-activation with PMA and ionomycin the level of secreted IL-10 was detected with the cell surface capture and detection antibodies by FACS. In the absence of zbtb20, there was a 50% decrease of Tregs that were able to produce IL-10 after a 3h stimulation (FIG. 6F). Thus, the development and function of these two T cell populations are dependent on the expression of zbtb20.

### Example 6: Loss of Zbtb20 in T Cells Impacts the Integrity of the Intestine

Alterations in the lymphocyte composition, including the reduction of IL-10 production might impact intestinal homeostasis, leading to inflammation resulting in tissue damage. The average length of the colons from zbtb20-cKO and WT mice was, however, the same (FIG. 6G). Histological assessment of colons collected from 8-week-old, naïve mice, showed subtle but consistent alterations in mucosal architecture in zbtb20-cKO mice as compared with WT, comprising of mild crypt hyperplasia, goblet cell loss, mucosal edema, as well as increased lymphocytic nodules in the lamina propria (FIGS. 6H, 6I). These findings suggested that the integrity of the epithelial barrier might be compromised in the absence of fully functional Zbtb20 expressing T cells. To test this, 14-week old zbtb20-cKO and WT mice were gavaged with a mixture of size-selective probes including creatinine, 4 kDa FITC dextran (FD4), and 70 kDa Rhodamine B dextran (RD70). Although all three probes (creatine, FD4, and RD70) were elevated in zbtb20-cKO mice as compared to matched WT controls (FIG. 6J), there was no change in the creatinine/RD70 or FD4/RD70 ratios (FIG. 13H). Combined, these data demonstrate an increase in the unrestricted pathway indicative of epithelial damage, which is consistent with the morphological changes observed in zbtb20-cKO mice.

### Example 7: Colitis is Exacerbated in Zbtb20 Conditional Knock Out Mice

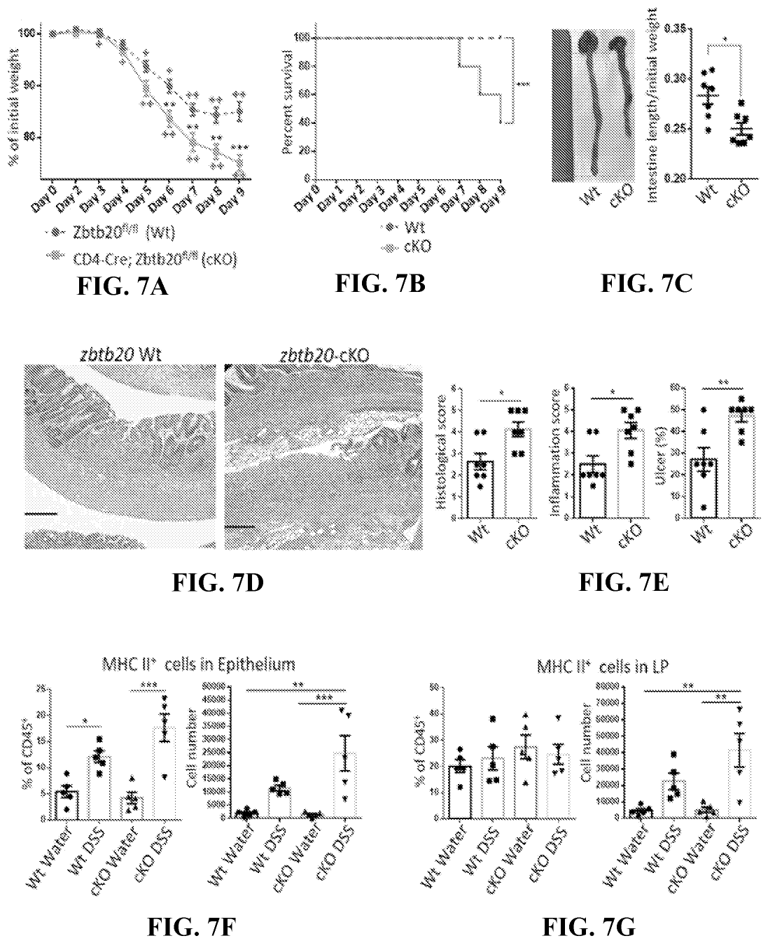

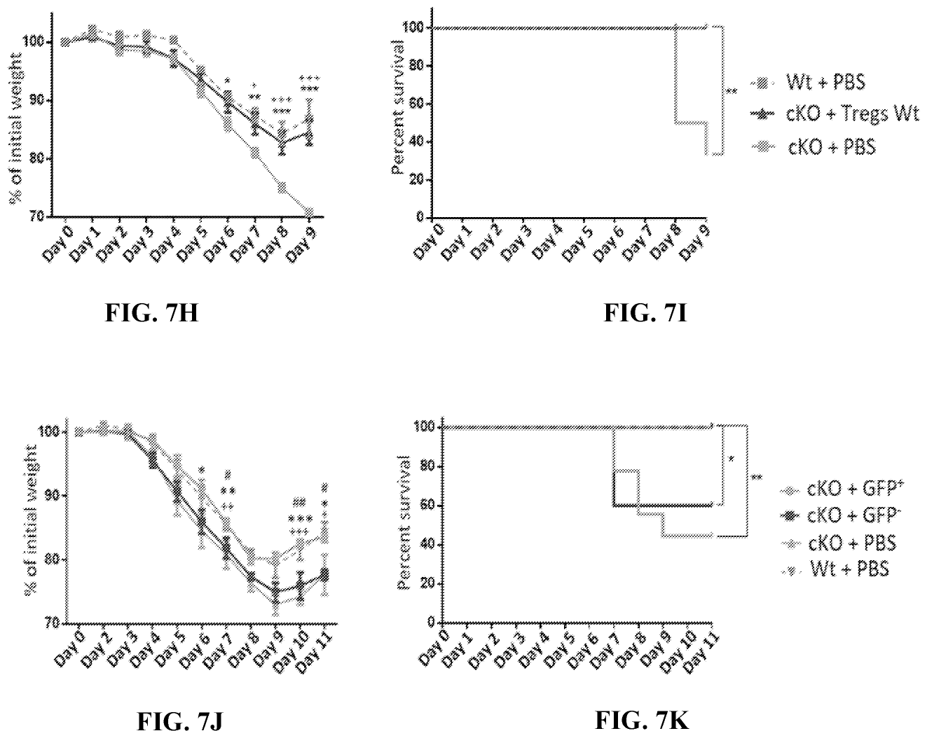

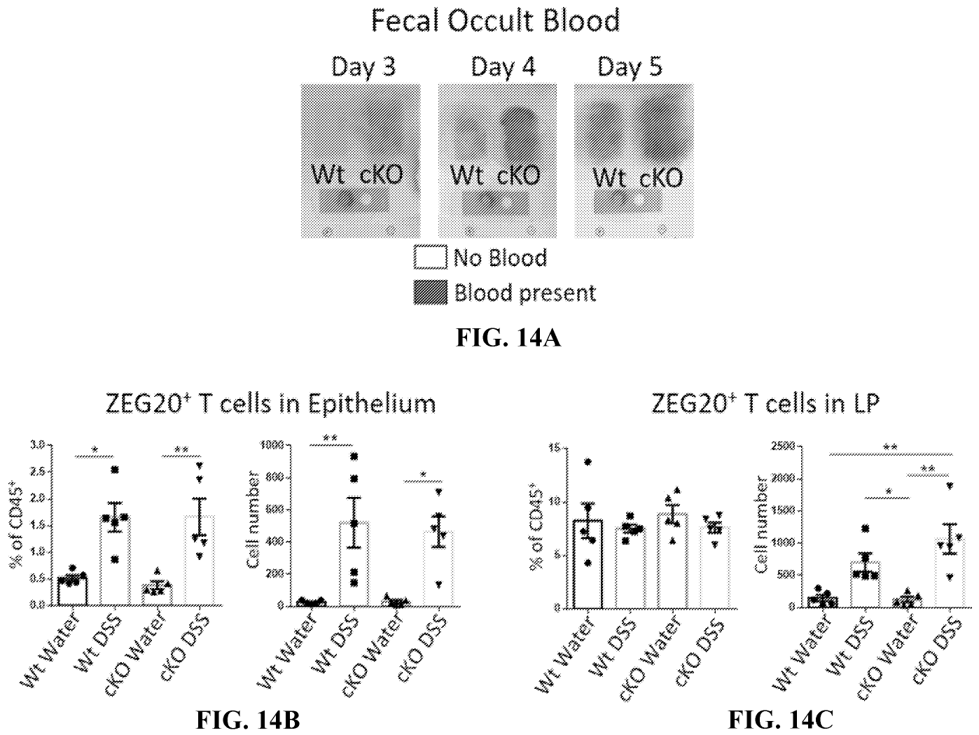

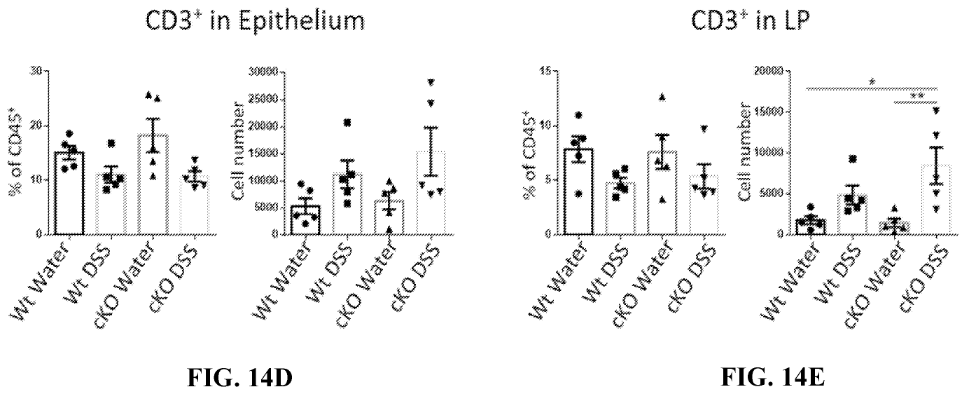

To further assess the requirement for Zbtb20 expressing T cells, zbtb20-cKO mice were challenged with 3% DSS in drinking water for 5 days followed by a 4-day recovery period. The zbtb20-cKO mice presented with more severe symptoms of colitis including greater loss of body weight (FIG. 7A). Moreover, occult blood was detected in the stool of zbtb20-cKO mice two days earlier than in WT which is consistent with the more severe intestinal inflammation observed in the absence of zbtb20 in T cells (FIG. 14A). Approximately 60% of zbtb20-cKO mice died prior to day 9 post-induction of colitis, whereas none of the wild type mice succumbed (FIG. 7B).

The colons of DSS-treated zbtb20-cKO mice that survived to the end of the recovery period were significantly shorter than WT mice (FIG. 7C) and exhibited more severe injury and inflammation (FIGS. 7D, 7E). Histological assessment of the colons showed substantial ulceration in zbtb20-cKO with approximately 50% of the colonic surface being an ulcer as compared to 25% in WT mice, which was mostly focal (FIG. 7D). Colons of both, Wt and zbtb20-cKO mice had atypical hyperplasia of the crypts, with crypt dilatation, and abscess. Lastly, scoring of inflammatory infiltrates revealed a high density of transmural lymphocyte infiltration (size of GALT>50%) and severe epithelial exfoliation in zbtb20-cKO mice, with less florid transmural inflammation (size of GALT 20-50%) and focal exfoliation observed in WT (FIG. 7E).

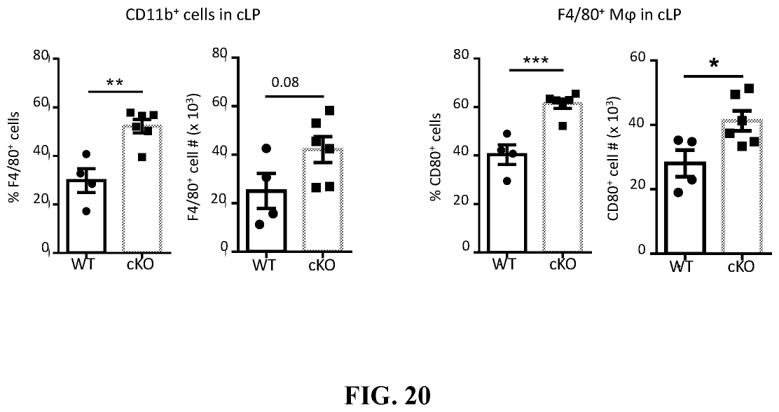

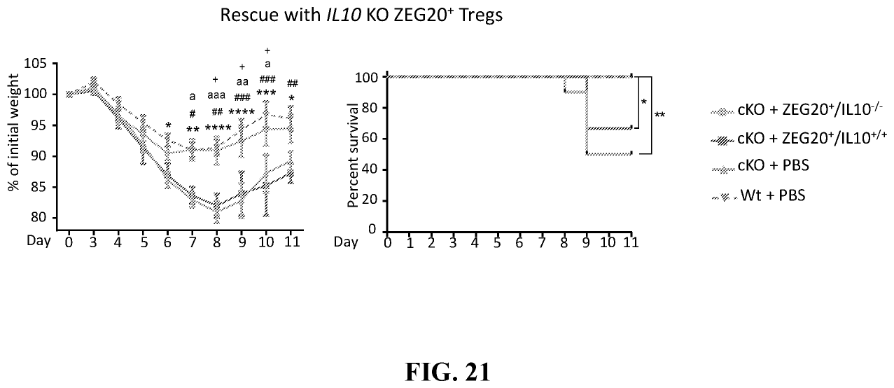

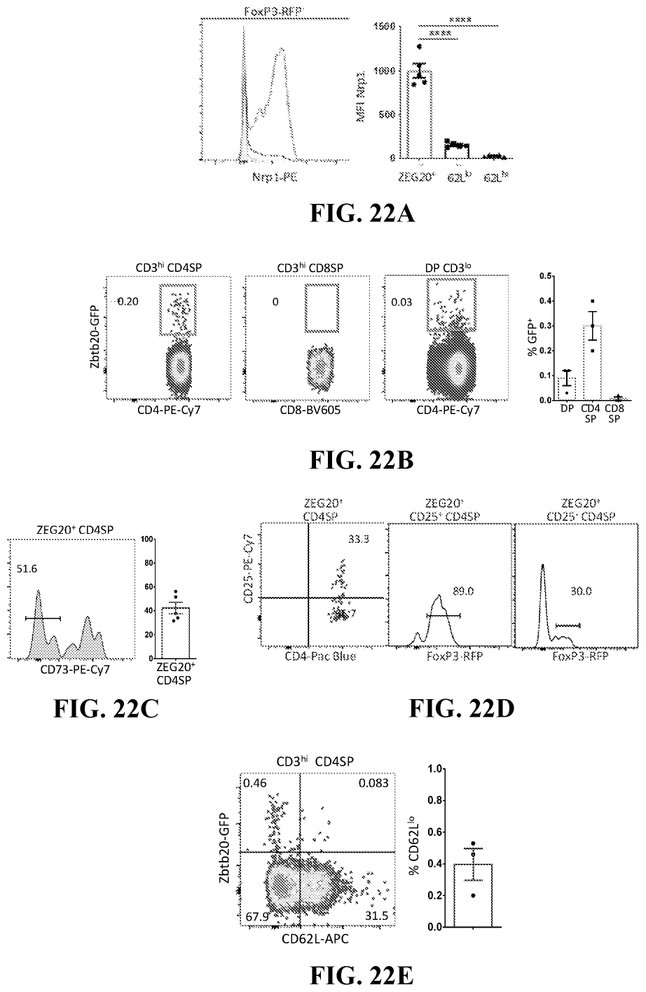

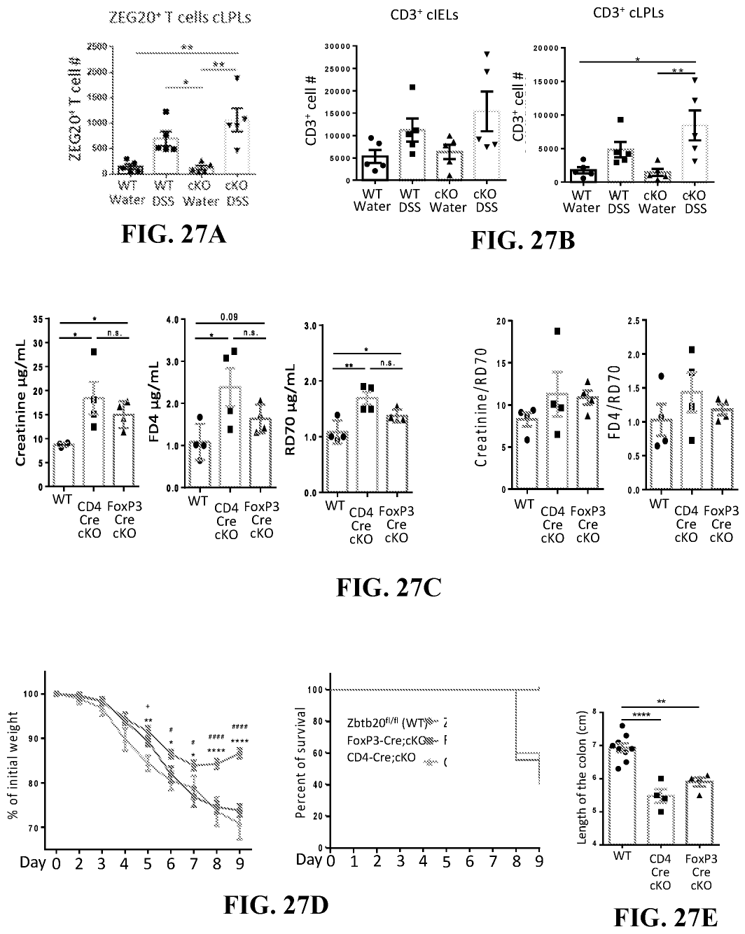

FACS analysis of leukocytes collected from the colons of mice receiving DSS showed a similar increase of ZEG20⁺ T cells and Zbtb20^{wannabe} T cells in both inflamed epithelium and LP (FIG. 27A)) and also a moderate increase in the number of non-Treg, non-Zbtb20-expressing CD3⁺ T cells in the cKO mice (FIG. 27B). These data suggested that the changes in function, rather than frequency, of the Zbtb20^{wannabe} T cells exacerbates the severity of colitis in zbtb20-cKO mice. An increase of MHC II⁺ myeloid cells and non-Treg, non-Zbtb20 expressing CD3⁺ T cells was also observed throughout the colon, but particularly in the LP in the zbtb20-cKO mice (FIGS. 7F, 7G, 14D, 14E). In particular, the cKO mice had a 2-fold increase of F4/80⁺ macrophages most of which were CD80⁺ proinflammatory cells (FIG. 20).

Studies next analyzed mice in which Zbtb20 was specifically deleted in Tregs by use of FoxP3-Cre mice (FoxP3-Cre;cKO mice). Results showed that these mice also had epithelial damage that allowed the contents of the intestinal lumen to leave via the unrestricted pathway (FIG. 27C). Deletion of Zbtb20 with FoxP3-Cre also resulted in more severe symptoms of colitis following treatment with DSS (FIG. 27D) leading to the death of 60% of the cKO mice. Furthermore, the surviving FoxP3-Cre;cKO mice had shorter colons (FIG. 27E). The consequences of deletion of Zbtb20 with FoxP3-Cre, overall were less severe than deletion with CD4-Cre, indicating that the Zbtb20-expressing CD4 T cells also play an active role in suppressing the inflammation. Collectively, without wishing to be bound by theory, these data show that Zbtb20 expressing T cells have potent protective abilities, as impairment of their function markedly increased the severity of DSS-induced injury and inflammation.

### Example 8: Adoptive Transfer of Zbtb20 Expressing Tregs Attenuates DSS-Induced Colitis

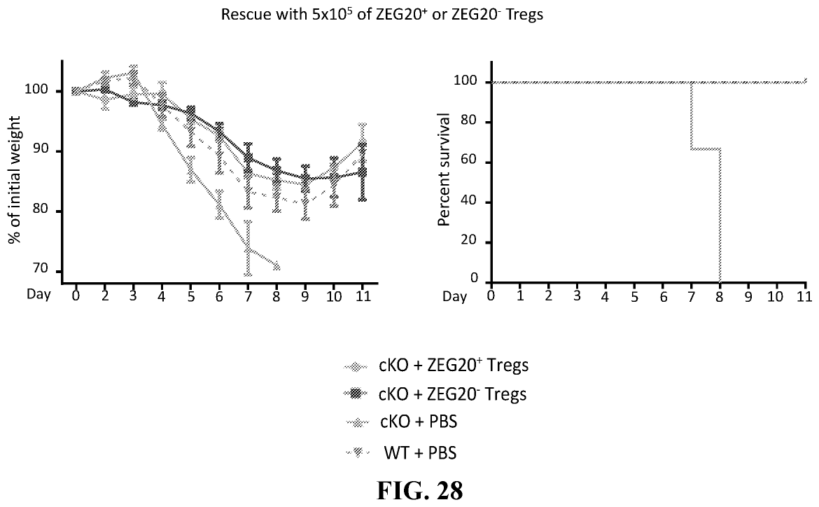

Mice treated with 3% DSS developed more severe colitis in the absence of zbtb20 expression in CD4⁺ T cells. However, it is not known if the damage to the intestine that results in increased susceptibility to colitis, accumulated from birth in zbtb20-cKO mice or if Zbtb20⁺ Tregs directly controlled the detrimental outcome of the disease. To test this, a series of adoptive transfer “rescue” experiments were performed where the zbtb20-cKO mice were i.p. injected with Tregs one day prior to DSS treatment. Transfer of 5×10⁵ total Tregs from WT mice mitigated the symptoms of the colitis in zbtb20-cKO mice, resulting in the progression of the disease that closely resembled WT mice (FIGS. 7H, 7I). Next, studies were carried out which adoptively transferred sorted ZEG20⁺ Tregs or ZEG20⁻ CD62L^{lo} Tregs into zbtb20-cKO mice one day before the treatment with 3% DSS. The mice that received the Zbtb20 expressing cells had mild colitis that closely resembled that observed in WT mice (FIG. 7J). All the mice that received ZEG20⁺ Tregs survived. At higher numbers (5×10⁵), non-Zbtb20 Tregs were able to rescue the cKO mice (FIG. 28). In contrast, mice that received ZEG20⁻ CD62L^{lo} Tregs had excessive weight loss and 40% succumbed to the disease (FIG. 7K). Altogether, these results show that Zbtb20 expressing Tregs are essential for controlling DSS-induced colitis, potentially due to enhanced immunosuppressive activity.

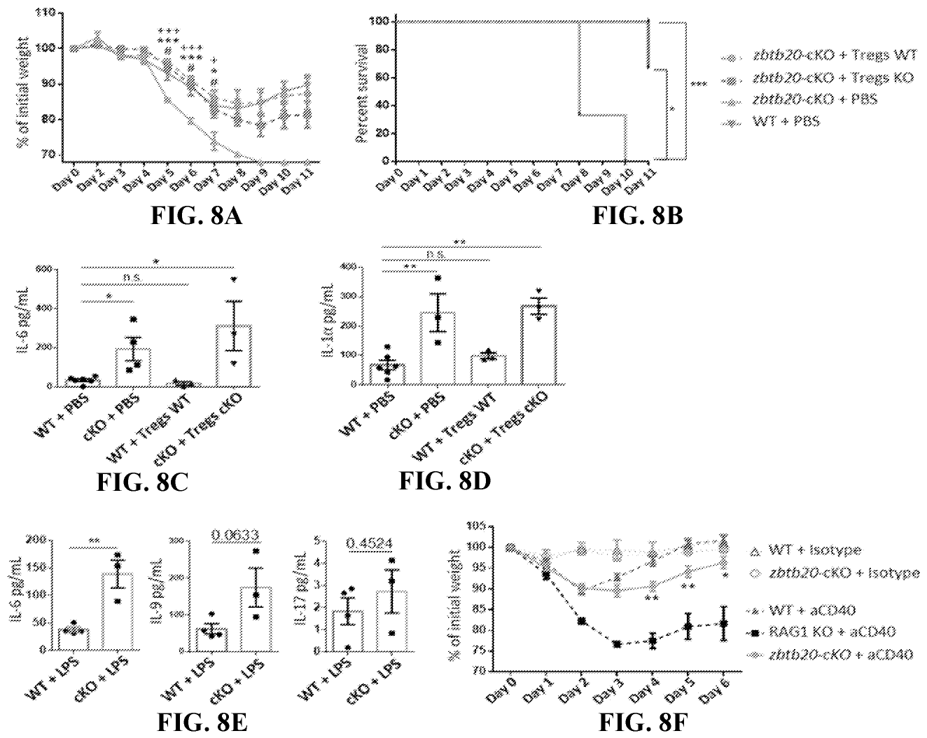

Lastly, total Tregs were collected from either zbtb20-cKO or WT mice and their ability to protect the cKO mice from DSS-induced colitis was observed. 5×10⁵ of total Tregs from either zbtb20-cKO or WT mice were i.p. injected into the cKO mice one day prior to treatment with 3% DSS. Although the increased weight loss observed in zbtb20-cKO mice that received Tregs from zbtb20-cKO did not reach statistical significance when compared to recipients of Tregs from WT mice, 40% of these mice succumbed to disease (FIGS. 8A, 8B). None of the mice receiving Tregs from WT mice died. These data suggest that even when sufficient numbers of Tregs are transferred to attenuate symptoms of colitis, the impaired function of Zbtb20 expressing Tregs prevents full recovery of the zbtb20-cKO mice. Therefore, even though Zbtb20 expressing T cells comprise just a small fraction of total Tregs, they have immunosuppressive abilities that complement and enhance the overall Treg population.

### Example 9: Myeloid Cell Expressed Cytokines are Increased During DSS-Induced Colitis in the Absence of Zbtb20 Expression in T Cells

To assess the nature of the immunological response that is controlled by Zbtb20 expressing Tregs, different subpopulations of Tregs were adoptively transferred into zbtb20-cKO mice, in which colitis was then induced, and serum collected for cytokine analysis. The zbtb20-cKO mice were injected with 5×10⁵ of total Tregs isolated from spleens of either WT or zbtb20-cKO mice. Control zbtb20-cKO and WT animals were injected with vehicle (PBS). One day after injection, DSS was administered to all four groups of mice. On day 9, serum was collected, and the level of different cytokines was assessed using CBA. zbtb20-cKO mice injected with vehicle had elevated levels of proinflammatory cytokines such as IL-6 and IL-1α when compared to WT mice, whereas zbtb20-cKO mice that received WT Tregs had low levels of these cytokines that were comparable to WT mice. The levels of the cytokines, however, remained elevated in the zbtb20-cKO mice that received the cKO Tregs (FIGS. 8C, 8D). Thus, it is possible that systemic inflammation in the mice with functionally impaired zbtb20 deficient Tregs caused higher mortality of zbtb20-cKO mice during the progression of colitis (FIG. 6B). Altogether these data further demonstrate that the Zbtb20 expression in a subset of Tregs is essential for controlling intestinal inflammation.

In IBD, mononuclear cells, which are predominantly macrophages, residing within the LP are the major source of IL-6 and IL-1α in the inflamed intestine. Thus, it was postulated that the exacerbated symptoms of colitis in zbtb20-cKO mice might be a result of the aberrant activation of myeloid cells. To test this hypothesis, WT and zbtb20-cKO mice were i.p. injected with 100 g LPS (Sigma-Aldrich), and serum was collected after 21h for cytokine analysis. The pro-inflammatory cytokines IL-6, IL-9, and IL-17 were found to be approximately 3-fold higher in zbtb20-cKO mice (FIG. 8E). To directly test a possible role for aberrant myeloid cell function in zbtb20-cKO, a model was utilized that induces colitis via the direct activation of CD40 expressing cells, which in the intestine, are mostly CX3CR1^{hi} macrophages. It has been shown that in RAG1 deficient mice, which lack T cells and B cells, activation of CD40 signaling causes severe colitis. To test the impact of the loss of zbtb20 in CD4⁺ T cells on activation of myeloid cells, 100 μg of the agonist anti-CD40 antibody, FKG45, or isotype control was i.p. injected into RAG1 KO, zbtb20-cKO, or WT mice. Progression of colitis was monitored by measuring body weight daily (FIG. 8F). As expected, RAG1 KO mice lost a substantial amount of weight, while WT mice were mostly unaffected. The zbtb20-cKO, however, also lost a significant amount of weight, suggesting that colitis was substantially worse as compared to WT mice. These data show that the small subset of Zbtb20 expressing T cells is essential for expanding the overall activity of the much larger Treg population and suggest that the role of Zbtb20 expressing T cells in the regulation of intestinal homeostasis is mediated by the control of intestinal myeloid cells.

### Example 10: Expression of ZBTB20 in Peripheral T Cells Induces Predicted Phenotypic Changes

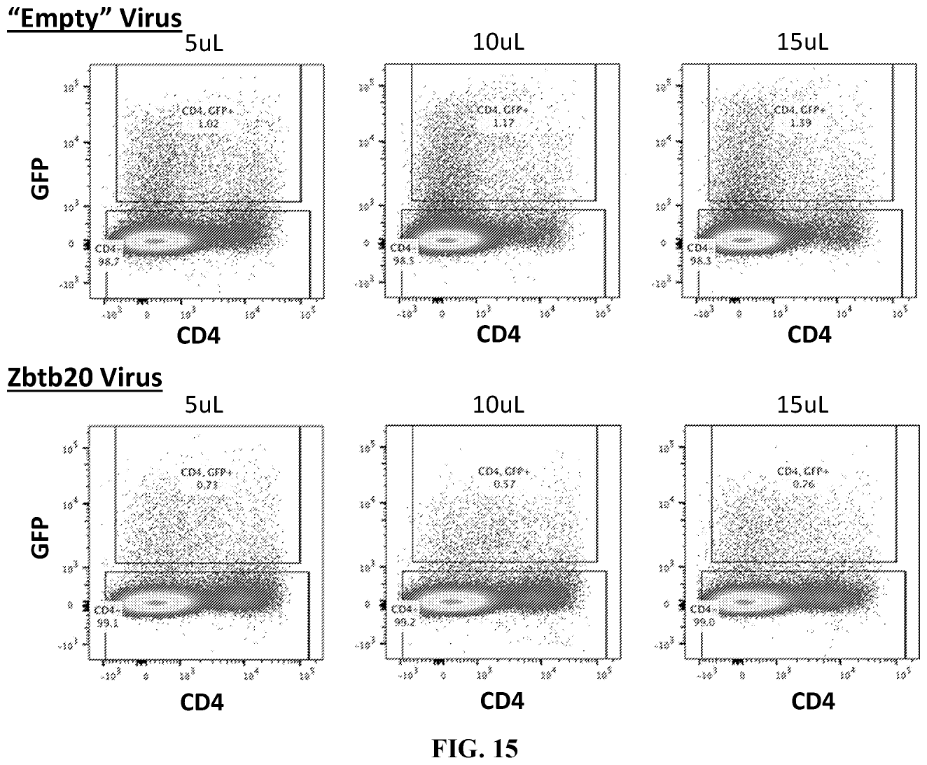

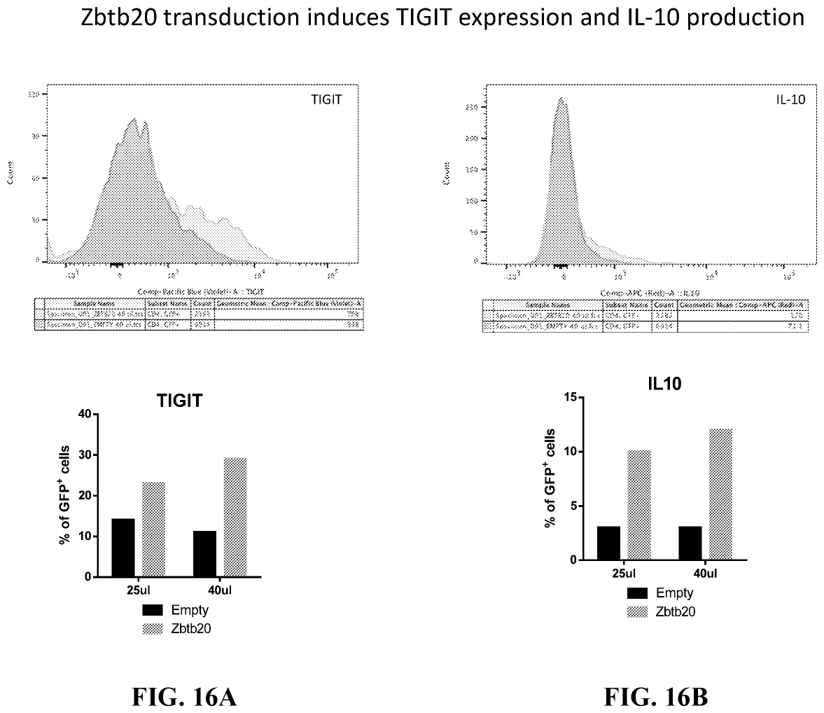

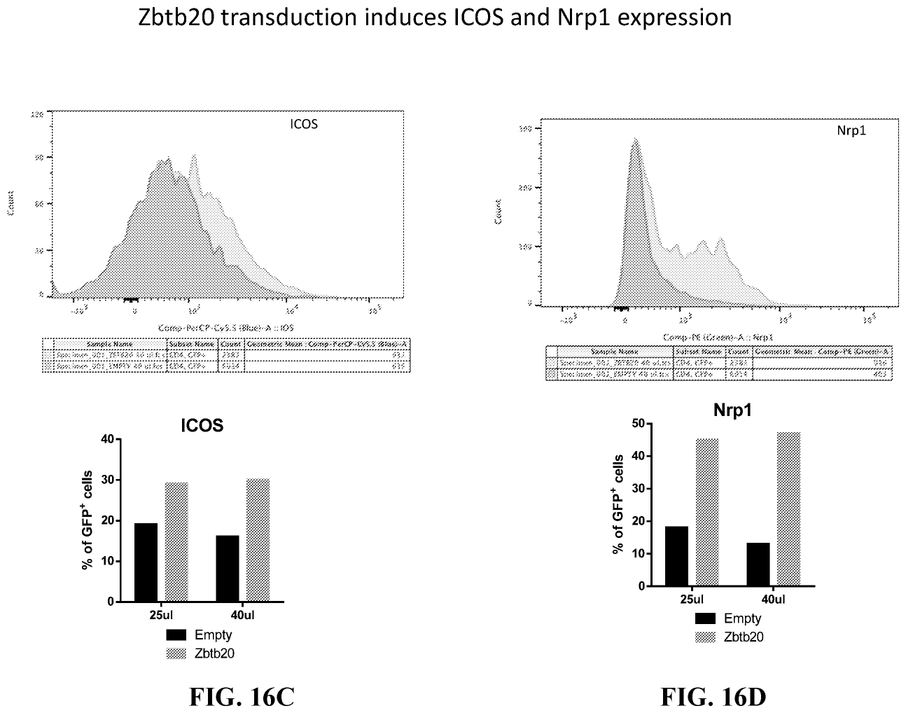

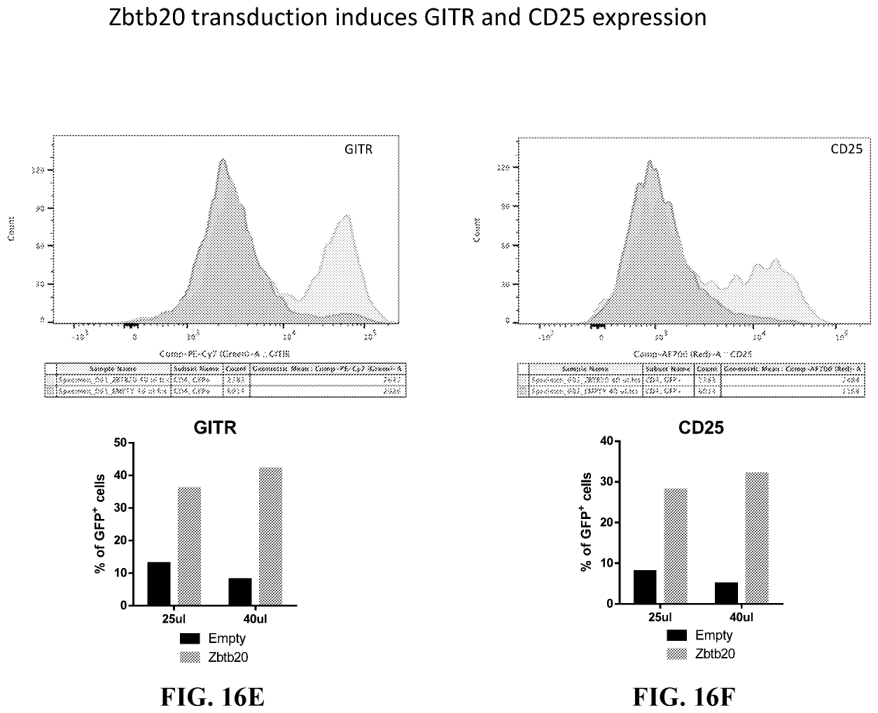

A series of studies was then performed in order determine whether expression of exogenous ZBTB20 protein in isolated peripheral T cells via viral transduction could result in phenotypic changes that resemble endogenous Zbtb20⁺ T cells. Here CD4⁺ were sorted from wildtype 8-week old mice via AutoMacs. Purified T cells were then activated with PMA/ionomycin treatment for 96 hours in a 96-well plate followed by transduction and reactivation with Zbtb20 viral vector or an empty viral vector control. Briefly, the transduction protocol utilized 8 μg/ml of Polybrine and anti-CD3/anti-CD28 beads at a 1:1 ratio. Cells were centrifuged at 22000 rpm for 90 minutes at 32° C. to increase transduction efficiency. FIG. 15 shows that the transduced vector expresses GFP and can be used to monitor the efficiency of transduction. T cells were transduce with “empty” vector (GFP only) or Zbtb20 expressing vector. These studies were performed using 5 μl, 10 μl, and 15 μl of virus, though subsequent studies used 25 μl and 40 μl. FIGS. 16A-16F demonstrate that Zbtb20 transduction results in the upregulation of phenotypic markers associated with endogenous Zbtb20⁺ T cells, including TIGIT (FIG. 16A), IL-10 (FIG. 16B), ICOS (FIG. 16C), Nrp1 (FIG. 16D), GITR (FIG. 16E), and CD25 (FIG. 16F). Without wishing to be bound by theory, these data indicate that ex vivo transduction of peripheral blood T cells to express Zbtb20 could be used to generate large numbers Zbtb20⁺ T cells that resemble endogenous Zbtb20⁺ T cells.

### Example 11: Zbtb20 Expression During Thymic Development

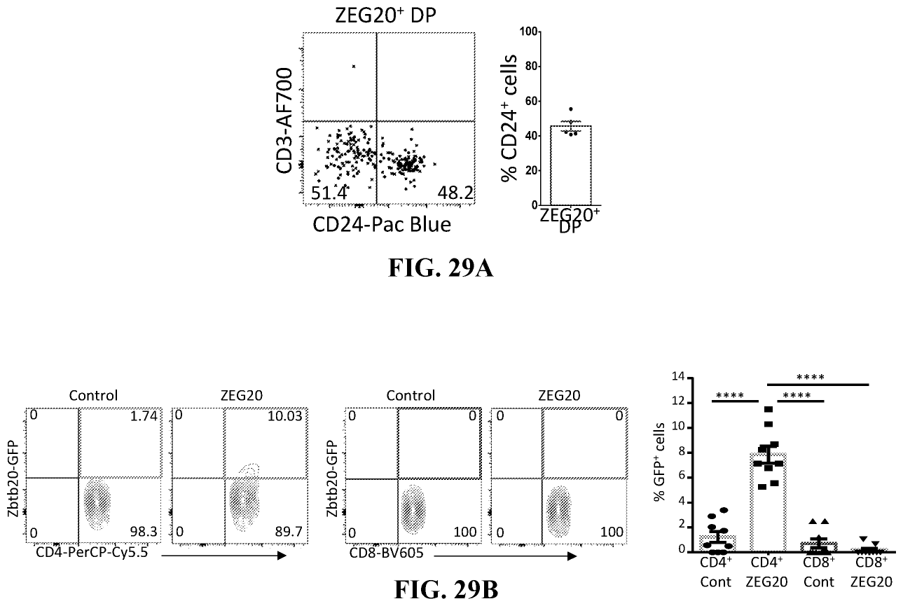

Without wishing to be bound by theory, the higher Neuropilin-1 (Nrp1) expression levels on spleen Zbtb20-expressing Tregs and T cells (FIGS. 17A, 17B) suggested their development in the thymus. FACS analysis showed that Zbtb20 expression was detected in CD3^{hi}CD4⁺ CD8⁻ single-positive cells (CD4SP), but not in CD8SP thymocytes (FIG. 8C). Zbtb20 was also expressed in CD3^{lo} CD4⁺ CD8⁺ CD24⁺ DP (double positive) thymocytes (FIGS. 8C, 29A). Peripheral Tregs that migrate back to the thymus tends to express CD73. Approximately half of the ZEG20⁺ CD4SP cells were negative for CD73 (FIG. 8D), which is comparable to the number previously reported for the total FoxP3+CD4SP population. Similar to spleen ZEG20⁺ cells, ˜30% of ZEG20⁺ CD4SP cells expressed CD25 (FIG. 8E) and nearly all of these cells expressed FoxP3 (FIG. 8E). Also, like in the spleen, ˜30% of the ZEG20⁺ CD25⁻ CD4SP cells expressed FoxP3 (FIG. 8E). ZEG20⁺ CD4SP thymocytes expressed low levels of CD62L suggesting that this phenotype was induced during development (FIG. 8F).

The induction of Zbtb20 in fetal thymic organ cultures (FTOC) was then assessed. Thymuses taken from mouse fetuses carrying the Zbtb20-eGFP reporter or controls were analyzed by FACS after 14 days of culture. Only CD4SP cells from the reporter thymuses expressed GFP, indicating that Zbtb20 had been induced (FIG. 29B). Cells that develop in FTOC may not reach full maturity, however, and therefore these data might not be consistent with what occurs in the intact mouse.

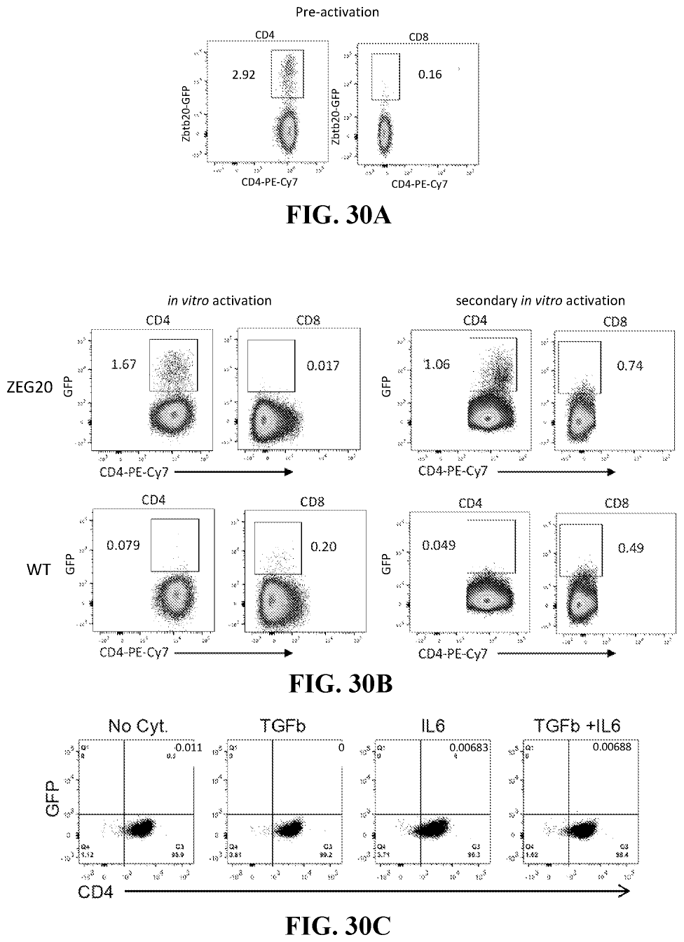

Zbtb20 was not induced in total spleen T cells or sorted ZEG20 negative CD4⁺ T cells by primary or secondary activation with antibodies against CD3 and CD28 in vitro (FIGS. 30A, 30B). Activation in the presence of exogenous TGFβ and IL-6, which induces FoxP3 expression, was also tested but this too had no impact on Zbtb20 expression (FIG. 30C). Zbtb20 was also not detected in CD8⁺ T cells.

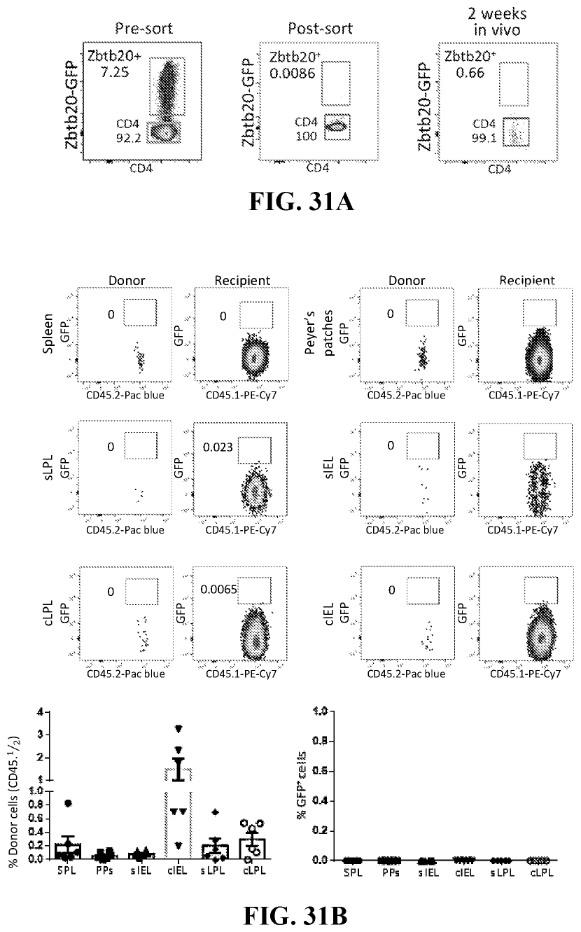

Allelically marked (CD45.2), FACS sorted ZEG20⁻ (CD62L^{hi}) CD4⁺ spleen T cells were adoptively transferred into CD45.1 (B6.SJL) host mice. After two weeks, lymphocytes were isolated from the Peyer's Patches, and transferred CD45.2⁺ T cells were analyzed by FACS. GFP expression was not detected in these cells (FIG. 31A). Lastly, we adoptively transferred FACS sorted ZEG20⁺ CD4SP thymocytes followed by induction of colitis with DSS. Ten days post-induction, spleen and intestinal T cells were analyzed by FACS (FIG. 31B). Induction of GFP in the donor cells was not detected. Combined, these data suggest that Zbtb20-expressing Tregs are a thymic-derived subset rather than a derivative of conventional Tregs that acquire these distinct features in the periphery.

### Example 11: Selected Discussion

A screen for new T cell effector populations identified two subsets of T cells defined by expression of the BTB-ZF gene, zbtb20. Here in the present disclosure, it is shown that both the Zbtb20⁺ Tregs (FoxP3+CD4⁺) and Zbtb20⁺ CD4⁺ (FoxP3⁻) T cells have distinct phenotypes, constitutively transcribe IL-10 mRNA and rapidly produce the cytokine following primary activation. The subsets are found in the thymus and spleen but are enriched in the intestine indicating a potential role in the regulation of intestinal homeostasis. Without wishing to be bound by theory, it was found that Zbtb20 expressing T cells were increased in frequency in response to experimental colitis.

To study a potential role for Zbtb20 expressing T cells during colitis, mice that had zbtb20 conditionally deleted in T cells were challenged with 3% DSS. zbtb20-cKO mice had substantially more intestinal inflammation and damage as assessed by histology, occult blood present days earlier in the stool, and substantially greater loss of body weight, all of which often culminated in increased mortality. Adoptive transfer of total Tregs from zbtb20 deficient mice failed to rescue zbtb20-cKO mice from death due to the severe colitis, demonstrating that non-Zbtb20 expressing Tregs were insufficient for full protection. Furthermore, the adoptive transfer of sorted Zbtb20 expressing Tregs prevented both the exacerbation of weight loss and mortality in zbtb20-cKO mice, whereas the transfer of non-Zbtb20 expressing Tregs did not. Combined, these data showed that protection from disease was directly attributable to Zbtb20 expressing Tregs and, moreover, was dependent on zbtb20 expression. The ability of Zbtb20 expressing Tregs to rescue zbtb20-cKO mice with acute colitis supports the idea that these cells have potent protective abilities that complement and enhance the overall Treg population.

Using FACS analysis the studies of the present disclosure showed that Zbtb20⁺ Tregs and Zbtb20⁺ CD4⁺ T cells have an activated phenotype (CD62L^{lo}) and higher levels of expression of the immunomodulatory molecules TIGIT, GITR, ICOS, and NRP1, which could contribute to their immunoregulatory abilities. Importantly, it was also shown that these cells have “pre-formed” IL-10 mRNA and can rapidly secrete IL-10 following stimulation. Thus, without wishing to be bound by theory, it is possible that this is a direct mechanism for the function of Zbtb20 expressing T cells since IL-10 plays a pivotal role in proper intestinal homeostasis. For example, IL-10 can hinder the proliferation and/or differentiation of several types of immune cells (e.g. DCs, B and T cells, Tregs, NK cells). IL-10 can also suppress the ability of T cells to produce cytokines both directly and indirectly through down-regulation of MHC II and co-stimulatory molecules (CD80, CD86) on antigen presenting cells (APCs) rendering them unable to activate CD4⁺ T cells. Hence, it is likely that continuous expression of IL-10 by Zbtb20 expressing T cells might alter the function of other immune cell types in the intestine. Such functionality would be analogous to the modification of CD8⁺ T cells' effector functions by IL-4 that is continuously released by some iNKT cells. Also, analogous to iNKT cells, the constitutively activated phenotype, and the presence of preformed cytokine message is already apparent in Zbtb20 expressing thymocytes. This suggests that these features are inherent to Zbtb20 expressing T cells, rather than peripherally acquired traits. The activated phenotype is muted in the absence of the gene, as is the ability to produce IL-10. Perhaps, therefore, Zbtb20 expressing Tregs can be considered yet another “innate-like” T cell subset.

Histological evaluation of colons collected from untreated zbtb20-cKO mice showed subtle but consistent changes in mucosal architecture, including mild crypt hyperplasia, goblet cell loss, edema and modest increased inflammatory cells. as early as 8-weeks of age. Epithelial damage, particularly as indicated by the increased intestinal permeability observed in the colon of zbtb20-cKO mice is a hallmark of IBD. Interestingly, increased intestinal permeability has been reported in a young patient with a positive family history of Crohn's disease almost a decade before the onset of the symptoms. Likewise, a permeability defect was observed in patients with quiescent IBD, but with a high risk of clinical relapse. Thus, without wishing to be bound by theory, it is possible that persistent subclinical inflammation observed in these patients, similar to what was found in zbtb20-cKO mice could be in part due to a reduced number and/or functional defect of Zbtb20 expressing T cells.

Induction of colitis in zbtb20-cKO and WT mice resulted in a rapid spike of IL-6 and IL-1α observed only in the serum of the cKO mice. Adoptive transfer of wild type Tregs, but not zbtb20 deficient Tregs prevented this burst of cytokines. An elevated level of IL-6 and IL-1α in individuals with IBD strongly correlates with the inflammatory activity and is attributed to a dysregulated inflammatory response. Furthermore, in mice, targeted deletion of IL-6 or injection of IL-6 neutralizing antibodies attenuated DSS-induced inflammation. Similarly, IL-1α neutralization reduces the severity of disease and increases the repair of the epithelial barrier following DSS-induced colitis.

The primary source of IL-6 and IL-1α in the inflamed intestine during IBD are monocytes residing within the LP and to some extent intestinal epithelial cells. FACS analysis of zbtb20-cKO mice with colitis showed an increase of MHC 11 cells both in LP and epithelium. This suggests that a loss of Zbtb20 in T cells compromises their function, leading to an uncontrolled expansion and/or activation of these myeloid cells and a burst of IL-6.

To further study the possibility that Zbtb20 expressing T cells control myeloid cells during colitis, zbtb20-cKO and WT mice were injected with an agonist, anti-CD40 antibody. CD40 is highly expressed by APCs in the colon LP, particularly CX3CR1^{hi} macrophages, and inducing CD40 signaling can trigger colitis driven by excessive production of proinflammatory cytokines by these cells. zbtb20-cKO mice injected with anti-CD40 developed colitis with substantially worse symptoms as compared to WT mice. These data strongly suggest a role for Zbtb20 expressing T cells in the regulation of intestinal homeostasis by acting on intestinal myeloid cells. Likely targets include CX3CR1^{hi} macrophages that both secrete and respond to IL-10. IL-10 production by CX3CR1^{hi} macrophages is, however, dispensable as specific deletion of IL-10 in these cells does not affect gut homeostasis. In contrast, mice with targeted deletion of IL-10Rα in CX3CR1^{hi} macrophages develop spontaneous colitis due to impairment of the function of these cells. Thus, the targeted deletion of zbtb20 leads not only to decreased IL-10 production by Zbtb20 expressing T cells but might also indirectly affect IL-10 secretion by other immune cells.

Overall in the present studies, BTB-ZF expression was explored at the single-cell level as a means to identify new effector T cell populations. This approach led to the discovery of two T cell populations: Zbtb20⁺ Tregs and Zbtb20⁺ CD4⁺ T cells. These cells have an activated phenotype (CD62L^{lo}, CD44^{hi}) and the ability to secrete IL-10 immediately after primary stimulation. These characteristics were found in antigen unexperienced Zbtb20 expressing T cells in the thymus, suggesting that this effector function is programmed during their development. These rare populations of T cells appear to have potent immunoregulatory abilities as the targeted deletion of zbtb20 in T cells made mice far more susceptible to the development of colitis. The impact was mostly cell intrinsic since disease symptoms were controlled by injection of Zbtb20⁺ Tregs but not Zbtb20⁻ Tregs. It is likely, however, that IL-10 produced by these cells impacts the function or activation status of other intestinal resident cells, such as CX3CR1^{hi} macrophages. Many aspects of Zbtb20 expressing T cells are analogous to NKT cells, suggesting that some “naïve” Tregs have innate-like effector functions that are not dependent upon differentiation in the periphery.

### Example 12: Role of Zbtb20+ Tregs in Cancer

The data presented in the present disclosure show that Zbtb20+ T cells are a novel type of regulatory T cell that are pivotal for controlling inflammation and preventing unwanted and/or unintended immune responses in the intestine. Zbtb20+ T cells, however, are not only found in the intestine—they are also present in other secondary lymphoid organs including the spleen. This suggested to us that Zbtb20+ Tregs might have roles in other diseases such as autoimmunity or immunity against cancer.

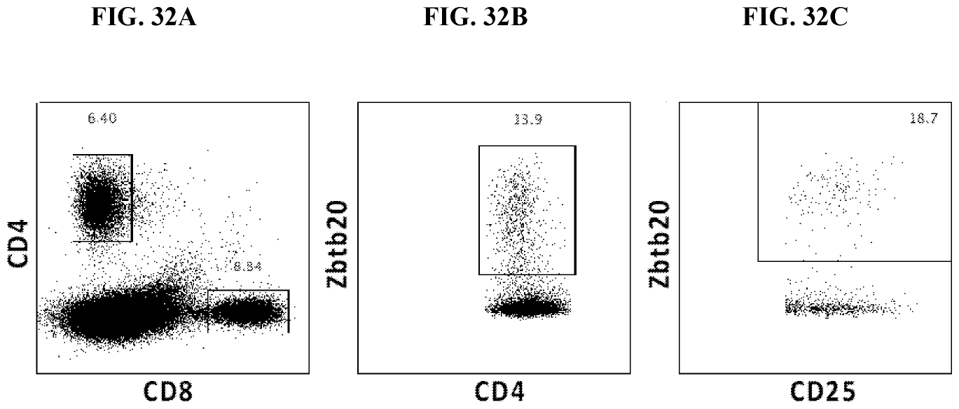

Given the well-known ability of Tregs to prevent effective immune responses to tumors. A series of studies were then undertaken in order to investigate whether, like in the intestine, Zbtb20+ Tregs have a significant impact on the anti-tumor response. The bladder cancer cell line, MB49, was first injected subcutaneously into the Zbtb20 reporter mice of the current disclosure, and tumor infiltrating lymphocytes (TIL) were assessed in established tumors two weeks later. These studies illustrated that CD4+ and CD8+ T cells had infiltrated the tumor (FIG. 32A). Interestingly, an average of 10.4% (SD+/−3; N=6) of the CD4+ T cells were Zbtb20+ (FIG. 32B) and among the CD25+CD4+ Tregs, an average of 19.8% (SD+/−6; N=6) were Zbtb20+ (FIG. 32C).

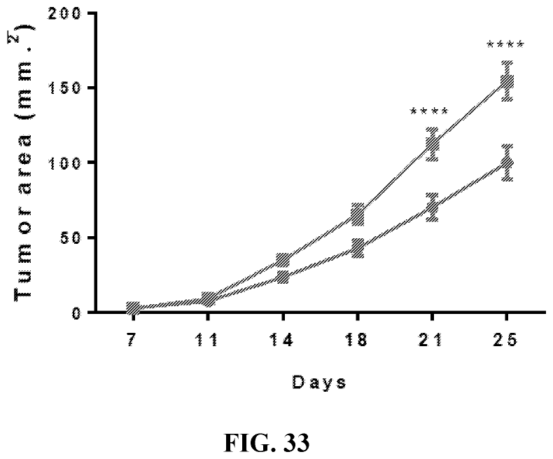

Next, tumor growth was analyzed in mice with Zbtb20 deleted in T cells (cKO mice). For these experiments, the melanoma cell line, DM4-BRaf^{V600E} was injected intradermally into cKO mice and age and sex matched WT littermates. Data from 4 independent experiments (N=24 WT and 21 cKO) show that the tumor grew substantially faster and bigger in the absence of Zbtb20+ T cells (FIG. 33). 28 days post implantation, tumors had grown an average of 50% larger in the cKO mice.

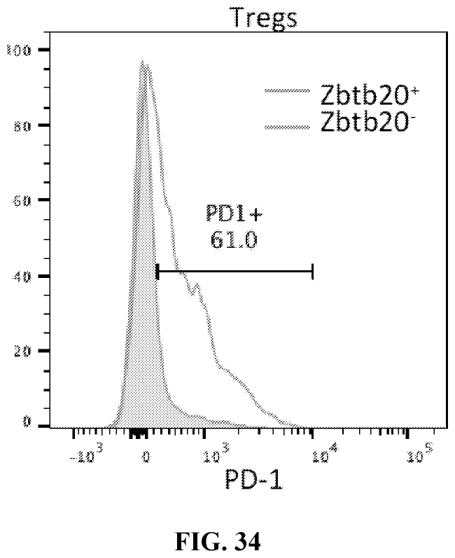

RNA-seq analysis of Zbtb20+ Tregs suggested that these cells expressed elevated levels of PD-1 as compared to non-Zbtb20 expressing Tregs. This result was confirmed by FACS (FIG. 34). When PD-1 expressed on an activated T cell interacts with its ligand, PD-L1, the T cell is signaled to shut down, or not respond; an interaction referred to as an immune checkpoint. Disrupting the PD1-PD-L1 interaction with a blocking monoclonal antibody (mAb), such as Keytruda (anti-PD1), prevents the checkpoint from functioning and allows the T cell to respond. In just ten years, treatment with checkpoint inhibitors has become a mainstay of anti-cancer therapies. Frustratingly, however, fewer than 30% of patients treated with checkpoint inhibitors have durable responses. Furthermore, efforts to stratify patients as responders or non-responders (i.e., who should or should not receive this type of treatment) has had only limited success. Finally, successful treatment of solid tumors with checkpoint inhibitors has proven to be very challenging. As a consequence, it is not surprising that there are thousands of active clinical trials are seeking both treatment options and the means to identify patients who will likely respond.

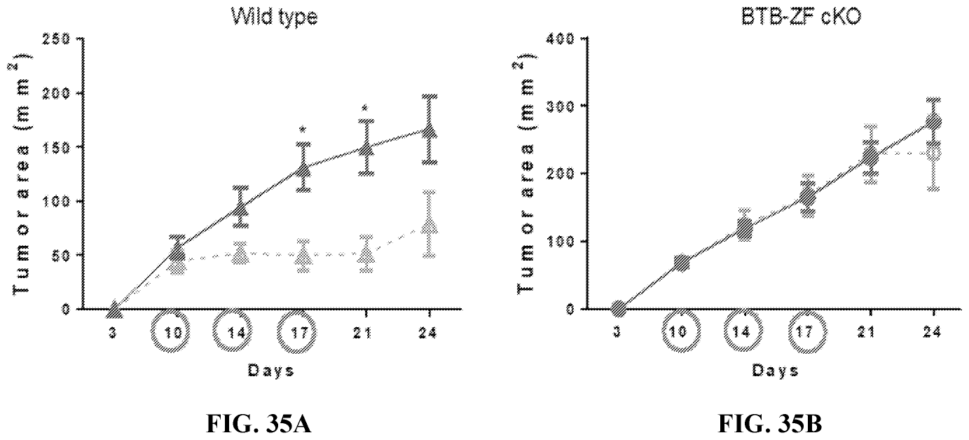

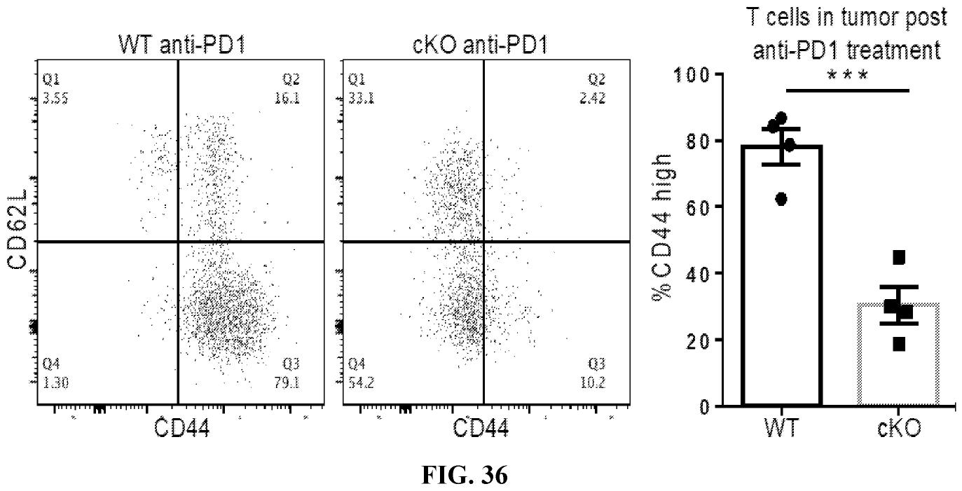

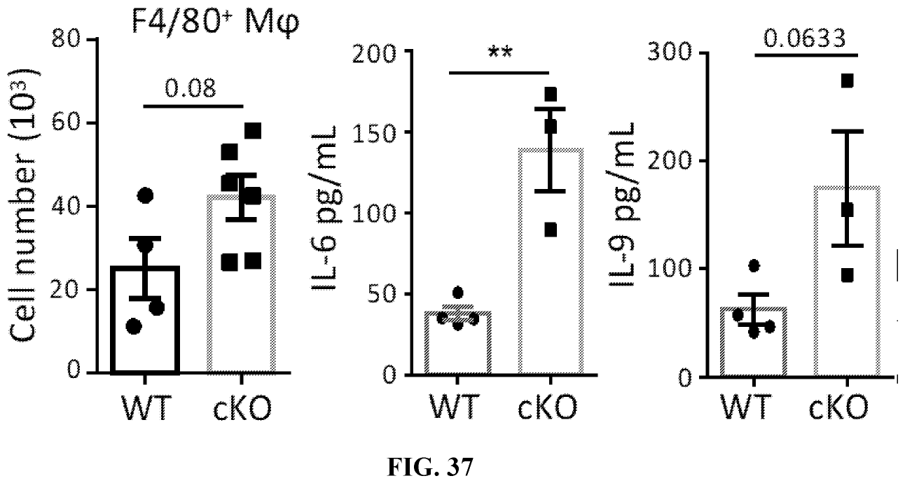

Since PD-1 is highly expressed on Zbtb20+ Tregs and Zbtb20+ Tregs are a substantial component of the tumor infiltration lymphocyte milieu, the possibility that anti-PD1 mAb checkpoint inhibitor treatment might be altered in Zbtb20 cKO mice was considered. To test the impact of anti-PD1 treatment, tumors were implanted and then, starting at day 10, anti-PD1 mAb was injected three times (day 10, 14 and 17 post implant). In wild type mice, as expected, the tumor grew much slower and was significantly smaller at the endpoint (FIG. 35). Remarkably, when the same experiment was done using Zbtb20 cKO mice, anti-PD1 treatment did not have any impact on tumor growth, as compared to untreated Zbtb20 cKO mice (FIG. 35). To begin to understand this outcome, TILs were isolated and analyzed by FACS. Nearly all T cells from tumors from WT mice treated with anti-PD1 had an activated CD44^{hi} phenotype (FIG. 36). In clear contrast, very few T cells isolated from the tumors in the anti-PD1 cKO mice were activated (FIG. 36). In summary, and without wishing to be bound by theory, these data show that the checkpoint inhibitor drug anti-PD1 (Keytruda) requires BTB-ZF transcription factor expressing T cells to function.

### ENUMERATED EMBODIMENTS

The following enumerated embodiments are provided, the number of which is not to be construed as designating levels of importance.

Embodiment 1 provides an isolated cell comprising a nucleic acid vector comprising a gene encoding the transcription factor ZBTB20 which is operably linked to a promoter.

Embodiment 2 provides the isolated cell of embodiment 1, wherein the promoter is constitutive.

Embodiment 3 provides the isolated cell of embodiment 1, wherein the promoter is inducible.

Embodiment 4 provides the isolated cell of any one of embodiments 1-4, wherein the promoter drives the expression of ZBTB20 such that the function of the isolated cell is altered.

Embodiment 5 provides the isolated cell of embodiment 4, wherein the expression of ZBTB20 results in enhanced IL-10 production by the isolated cell as compared to a cell not comprising the nucleic acid vector.

Embodiment 6 provides the isolated cell of any one of embodiments 1-5, wherein the cell is a T cell.

Embodiment 7 provides the isolated cell of embodiment 6, wherein the T cell is a regulatory T cell.

Embodiment 8 provides the isolated cell of any one of embodiments 1-7, wherein the cell is derived from a mammal.

Embodiment 9 provides the isolated cell of any one of embodiments 1-8, wherein the cell is derived from a mouse.

Embodiment 10 provides the isolated cell of any one of embodiments 1-8, wherein the cell is derived from a human.

Embodiment 11 provides a therapeutic composition comprising an effective amount of the isolated cell of any one of embodiments 1-10 and a pharmaceutically acceptable carrier.

Embodiment 12 provides a method for treating, ameliorating, and/or preventing an inflammatory disease in a subject in need thereof, the method comprising administering to the subject an effective amount of the isolated cell of any one of embodiments 1-10, thereby treating, ameliorating, and/or preventing the inflammatory disease.

Embodiment 13 provides the method of embodiment 12, wherein the inflammatory disease is a gastrointestinal inflammatory disease.

Embodiment 14 provides the method of embodiment 13, wherein the gastrointestinal inflammatory disease is selected from the group consisting of inflammatory bowel disease (IBD), Crohn's disease, and ulcerative colitis.

Embodiment 15 provides the method of any one of embodiments 12-14, wherein the cells are administered via a route selected from the group consisting of intravenous, intraperitoneal, intramuscular, subcutaneous, and implantation.

Embodiment 16 provides the method of any one of embodiments 12-15, wherein the cells are autologous to the subject.

Embodiment 17 provides the method of any one of embodiments 12-15, wherein the cells are heterologous to the subject.

Embodiment 18 provides a method of treating, ameliorating, and/or preventing an inflammatory disease in a subject in need thereof, the method comprising:
a. isolating a cell from the subject,
b. contacting the cell with a nucleic acid vector encoding ZBTB20 such that expression of ZBTB20 protein is elevated in the cell as compared to uncontacted cells, thereby inducing an anti-inflammatory function in the cell, and
c. administering the contacted cell to the subject thereby treating, ameliorating, and/or preventing the inflammatory disease.

Embodiment 19 provides the method of embodiment 18, wherein the inflammatory disease is a gastrointestinal inflammatory disease.

Embodiment 20 provides the method of embodiment 19, wherein the gastrointestinal inflammatory disease is selected from the group consisting of inflammatory bowel disease (IBD), Crohn's disease, and ulcerative colitis.

Embodiment 21 provides the method of any one of embodiments 18-20, wherein the cell is a T cell.

Embodiment 22 provides the method of embodiment 21, wherein the T cell is a regulatory T cell.

Embodiment 23 provides the method of any one of embodiments 18-22, wherein the anti-inflammatory function of the cell results from elevated expression of IL-10 by the cell.

Embodiment 24 provides the method of any one of embodiments 18-23, wherein the altered cells are administered via a route selected from the group consisting of intravenous, intraperitoneal, intramuscular, subcutaneous, and implantation.

Embodiment 25 provides a method of determining the risk of developing an inflammatory disease in a subject, the method comprising:
a. obtaining a tissue sample from the subject,
b. assessing the level of ZBTB20 expression in a cell of the sample, and
c. comparing the level of ZBTB20 expression to a baseline expression level established from normal tissue which does not present the inflammatory disease;
wherein ZBTB20 levels in the tissue sample that is lower than the baseline expression level represents an increased risk of the subject developing the inflammatory disease.

Embodiment 26 provides the method of embodiment 25, wherein the inflammatory disease is a gastrointestinal inflammatory disease.

Embodiment 27 provides the method of embodiment 26, wherein the inflammatory disease is selected from the group consisting of inflammatory bowel disease (IBD), Crohn's disease, and ulcerative colitis.

Embodiment 28 provides a method of determining whether a cancer patient is a candidate for cancer treatment with anti-PD-1 therapy, the method comprising:
a. obtaining a tumor sample from the patient,
b. assessing the level of ZBTB20 expression in a cell of the tumor sample, and
c. comparing the level of ZBTB20 expression in the patient's tumor sample to a baseline expression level established from a tumor which was successfully treated with anti-PD-1 therapy;
   wherein, if the patient's tumor sample has ZBTB20 levels that are lower than the baseline expression level, the patient is not a candidate for anti-PD-1 therapy.

Embodiment 29 provides the method of embodiment 28, wherein the cancer is a solid tumor.

Embodiment 30 provides the method of any one of embodiments 28-29, wherein the cancer is selected from the group consisting of melanoma, head and neck cancer, non-small cell lung cancer, bladder cancer, and microsatellite unstable cancers.

Embodiment 31 provides the method of any one of embodiments 28-30, wherein the anti-PD-1 therapy is an antibody blockade therapy.

Embodiment 32 provides the method of embodiment 31, wherein the antibody targets PD-1.

Embodiment 33 provides the method of embodiment 31, wherein the antibody targets PD-L1.

Embodiment 34 provides a method of immunotherapy for cancer for use in a patient in need thereof, the method comprising:
a. isolating an immune cell from the patient,
b. contacting the patient's immune cell with a nucleic acid vector encoding ZBTB20 such that expression of ZBTB20 protein is elevated in the patient's immune cell as compared to uncontacted immune cells, and
c. administering the contacted immune cell to the patient thereby treating or ameliorating the cancer.

Embodiment 35 provides the method of embodiment 34, further comprising administering to the subject anti-PD-1 therapy.

Embodiment 36 provides the method of embodiment 35, wherein the patient has a better cancer treatment response to the anti-PD-1 therapy than in the absence of being administered the contacted immune cell.

Embodiment 37 provides the method of any one of embodiments 35-36, wherein the anti-PD-1 therapy is an antibody.

Embodiment 38 provides the method of embodiment 37, wherein the anti-PD-1 therapy is an antibody specific for PD-1.

Embodiment 39 provides the method of embodiment 37, wherein the anti-PD-1 therapy is an antibody specific for PD-L1.

Embodiment 40 provides the method of any one of embodiments 34-39, wherein the immune cell is a T cell.

Embodiment 41 provides the method of embodiment 40, wherein the T cell is a CD4+ T cell.

Embodiment 42 provides the method of embodiment 40, wherein the T cell is a CD8+ T cell.

Embodiment 43 provides the method of embodiment 40, wherein the T cell is a mixture of CD4+ and CD8+ T cells.

Embodiment 44 provides the method of any one of embodiments 34-43, wherein the cancer is a solid cancer.

Embodiment 45 provides the method of any one of embodiments 34-44, wherein the cancer is selected from the group consisting of melanoma, head and neck cancer, non-small cell lung cancer, bladder cancer, and microsatellite unstable cancers.

### OTHER EMBODIMENTS

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. An isolated cell comprising a nucleic acid vector comprising a gene encoding the transcription factor ZBTB20 which is operably linked to a promoter, wherein:
a. the promoter is constitutive;
b. the promoter is inducible;
c. the promoter drives the expression of ZBTB20 such that the function of the isolated cell is altered;
d. the cell is derived from a mammal; and
e. the expression of ZBTB20 results in enhanced IL-10 production by the isolated cell as compared to a cell not comprising the nucleic acid vector.

2.-5. (canceled)

6. The isolated cell of claim 1, wherein the cell is a T cell or a regulatory T cell.

7. (canceled)

8. The isolated cell of claim 1, wherein the cell is derived from a mammal.

9. The isolated cell of claim 1, wherein the cell is derived from a human or a mouse.

10. (canceled)

11. A therapeutic composition comprising an effective amount of the isolated cell of claim 1 and a pharmaceutically acceptable carrier.

12. A method for treating, ameliorating, and/or preventing an inflammatory disease in a subject in need thereof, the method comprising administering to the subject an effective amount of the isolated cell of claim 1, thereby treating, ameliorating, and/or preventing the inflammatory disease, wherein:
a. the inflammatory disease is a gastrointestinal inflammatory disease;
b. the cells are administered via a route selected from the group consisting of intravenous, intraperitoneal, intramuscular, subcutaneous, and implantation;
c. the cells are administered via a route selected from the group consisting of intravenous, intraperitoneal, intramuscular, subcutaneous, and implantation; or
d. the cells are autologous or heterologous to the subject.

13. (canceled)

14. The method of claim 12, wherein the gastrointestinal inflammatory disease is selected from the group consisting of inflammatory bowel disease (IBD), Crohn's disease, and ulcerative colitis.

15.-17. (canceled)

18. A method of treating, ameliorating, and/or preventing an inflammatory disease in a subject in need thereof, the method comprising:
a. isolating a cell from the subject,
b. contacting the cell with a nucleic acid vector encoding ZBTB20 such that expression of ZBTB20 protein is elevated in the cell as compared to uncontacted cells, thereby inducing an anti-inflammatory function in the cell, and
c. administering the contacted cell to the subject thereby treating, ameliorating, and/or preventing the inflammatory disease;
wherein the inflammatory disease is a gastrointestinal inflammatory disease;
wherein the cell is a T cell or a regulatory T cell;
wherein the anti-inflammatory function of the cell results from elevated expression of IL-10 by the cell; or
wherein the altered cells are administered via a route selected from the group consisting of intravenous, intraperitoneal, intramuscular, subcutaneous, and implantation.

19. (canceled)

20. The method of claim 18, wherein the gastrointestinal inflammatory disease is selected from the group consisting of inflammatory bowel disease (IBD), Crohn's disease, and ulcerative colitis.

21.-24. (canceled)

25. A method of determining the risk of developing an inflammatory disease in a subject, the method comprising:
a. obtaining a tissue sample from the subject,
b. assessing the level of ZBTB20 expression in a cell of the sample, and
c. comparing the level of ZBTB20 expression to a baseline expression level established from normal tissue which does not present the inflammatory disease;
wherein ZBTB20 levels in the tissue sample that is lower than the baseline expression level represents an increased risk of the subject developing the inflammatory disease;
wherein the inflammatory disease is a gastrointestinal inflammatory disease; or
wherein the inflammatory disease is selected from the group consisting of inflammatory bowel disease (IBD), Crohn's disease, and ulcerative colitis.

26.-27. (canceled)

28. A method of determining whether a cancer patient is a candidate for cancer treatment with anti-PD-1 therapy, the method comprising:
a. obtaining a tumor sample from the patient,
b. assessing the level of ZBTB20 expression in a cell of the tumor sample, and
c. comparing the level of ZBTB20 expression in the patient's tumor sample to a baseline expression level established from a tumor which was successfully treated with anti-PD-1 therapy;
wherein, if the patient's tumor sample has ZBTB20 levels that are lower than the baseline expression level, the patient is not a candidate for anti-PD-1 therapy;
wherein the cancer is a solid tumor;
wherein the cancer is selected from the group consisting of melanoma, head and neck cancer, non-small cell lung cancer, bladder cancer, and microsatellite unstable cancers;
wherein the anti-PD-1 therapy is an antibody blockade therapy; or
wherein the antibody targets PD-1 or PD-L1.

29.-33. (canceled)

34. A method of immunotherapy for cancer for use in a patient in need thereof, the method comprising:
a. isolating an immune cell from the patient,
b. contacting the patient's immune cell with a nucleic acid vector encoding ZBTB20 such that expression of ZBTB20 protein is elevated in the patient's immune cell as compared to uncontacted immune cells, and
c. administering the contacted immune cell to the patient thereby treating or ameliorating the cancer;
wherein the method further comprises administering to the patient an anti-PD-1 therapy;
wherein the anti-PD-1 therapy is an antibody;
wherein the anti-PD-1 therapy is an antibody specific for PD-1 or PD-L1; or
wherein the patient has a better cancer treatment response to the anti-PD-1 therapy than in the absence of being administered the contacted immune cell.

35.-39. (canceled)

40. The method of claim 34, wherein the immune cell is a T cell.

41. The method of claim 40, wherein the T cell is selected from the group consisting of a CD4+ T cell, a CD8+ cell, and a mixture of CD4+ and CD8+ T cells.

42.-43. (canceled)

44. The method of claim 34, wherein the cancer is a solid cancer.

45. The method of claim 34, wherein the cancer is selected from the group consisting of melanoma, head and neck cancer, non-small cell lung cancer, bladder cancer, and microsatellite unstable cancers.
